(19) United States
(12) Patent Application Publication
(10) Pub. No.: US 2013/0261133 A1
(43) Pub. Date: Oct. 3, 2013

(54) Therapeutic Compounds for Protozoal and Microbial Infections and Cancer

(71) Applicant: THE UNIVERSITY OF MONTANA, Missoula, MT (US)

(72) Inventors: John Howard Hoody, Missoula, MT (US); David Brian Bolstad, Seattle, WA (US)

(73) Assignee: The University of Montana, Missoula, MT (US)

(21) Appl. No.: 13/803,648

(22) Filed: Mar. 14, 2013

Related U.S. Application Data
(60) Provisional application No. 61/616,291, filed on Mar. 27, 2012.

(51) Int. Cl. C07D 307/68; C07C 233/71; C07C 235/48; C07C 219/20; C07C 311/04; C07D 241/24; C07C 33/048; C07C 215/24; C07C 271/16; C07D 261/18; C07D 213/81; C07C 69/16; C07C 309/66

(52) U.S. Cl. CPC C07D 307/68; C07C 69/16; C07C 233/71; C07C 235/48; C07C 219/20; C07C 311/04; C07C 309/66; C07C 33/048; C07C 215/24; C07C 271/16; C07D 261/18; C07D 213/81; C07D 241/24
USPC 514/255.06; 560/262; 560/250; 514/546; 514/517; 558/52; 568/857; 514/738; 514/667; 564/503; 564/186; 514/617; 514/478; 560/160; 514/378; 548/248; 546/323; 514/354; 544/406; 549/487; 514/471

(57) ABSTRACT

The compounds of the invention exhibit antiprotozoal, antimicrobial, and anticancer properties that are useful for the treatment or prevention of infections or cancer in a patient (e.g., a human). For example, the compounds and methods described herein can be used for the treatment or prevention of protozoal infections such as leishmaniasis, malaria, and trypanosoma infections, bacterial infections such as S. aureus and C. albicans, and cancers such as breast, colon, lung, or prostate cancer. The invention further provides methods of synthesizing such compounds as well as kits useful for administering the compounds.

### CROSS-REFERENCE TO RELATED APPLICATIONS

This application claims the benefit of the filing date of U.S. Provisional Patent Application Ser. No. 61/616,291, filed Mar. 27, 2012, the disclosure of which is incorporated by reference in its entirety.

### BACKGROUND OF THE INVENTION

Leishmaniasis is one of several tropical diseases classified as a “neglected tropical disease.” Its global prevalence is 12 million people with an estimated at-risk population of 350 million people. Current treatments include antimonials, amphotericin B, pentamidine, and miltefosine. Access to these medications is often limited in the impoverished nations that Leishmaniasis predominantly affects. Drug resistance and toxicity are also significant drawbacks to the current treatments. Therefore, there is an immediate need for the development of novel therapeutic molecules and strategies to combat Leishmaniasis.

In 2007, Senn et al. reported the isolation of four polyacetylene compounds from the Tanzanian medicinal plant Cussonia zimmermannii (Antiprotozoal polyacetylenes from the Tanzanian Medicinal Plan Cussonia zimmermannii, J. Nat. Prod. 70:1565 (2007)). Three of the four compounds displayed activity against Trypanosoma brucei rhodesiense, Trypanosoma cruzi, Plasmodium falciparum, and Leishmania donovani. Of these four compounds identified by Senn, 8-hydroxyheptadeca-1-ene-4,6-diyn-3-yl acetate had the lowest IC₅₀ (0.32 μM) and the highest selectivity index (37; IC₅₀ for rat skeletal myoblasts/IC₅₀ for L. donovani) when screened against Leishmania donovani in infected macrophages.

### SUMMARY OF THE INVENTION

In general, the present invention is based on the discovery of compounds that exhibit antiprotozoal, antimicrobial, and anticancer properties. These compounds can be used clinically to treat or prevent infirmary in a patient (e.g. a human) caused by protozoal (e.g., leishmaniasis, malaria, or Chagas disease) or bacterial infection or cancer. Accordingly, in a first aspect, the invention provides compounds presented and defined by the following formula

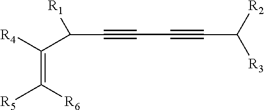

or a salt, ester or prodrug thereof, wherein
R₁ and R₂ vary independently and are selected from the group consisting of a hydrogen, halogen, hydroxyl, acetoxy, thiol, cyanide, azide, chlorosuccinimide, thiocyanate, amine, NHR₇, NR₇R₈, NR₇(C═O)R₈, C(═O)R₇, C(═O)NR₇R₈, NO₂, NH(SO₂)R₇, S(C═O)R₇, SO₂(N)R₇R₈, CO₂R₇, OR₇, OSO₂R₇, NR₇CO₂R₈, OC(═O)R₇, OC(═O)NR₇R₈, piperadino, pyrrolidino, piperazino, azetidino, morpholino, thiomorpholino, (C₁-C₂₀)alkyl, aryl(C₁-C₂₀)alkyl, and aryl, wherein any alkyl or aryl may be optionally substituted with one or more groups selected from R₂, and any alkyl group may optionally include one or more double or triple bonds within its carbon chain; and
R₃, R₄, R₅, R₆, R₇, and R₈ vary independently and are selected from the group consisting of a hydrogen, halogen, (C₁-C₂₀)alkyl, aryl(C₁-C₂₀)alkyl, and aryl, wherein any alkyl or aryl may be optionally substituted with one or more groups selected from R₂, and any alkyl or aryl alkyl group may optionally include one or more double or triple bonds within its carbon chain.

In one embodiment, the compound is (3S,8R)-8-hydroxyheptadeca-1-en-4,6-diyn-3-yl acetate; (3R,8R)-8-hydroxyheptadeca-1-en-4,6-diyn-3-yl acetate; (3R,8S)-8-hydroxyheptadeca-1-en-4,6-diyn-3-yl acetate; (3S,8R)-8-hydroxyhexadeca-1-en-4,6-diyn-3-yl acetate; (3S,8S)-8-hydroxyhexadeca-1-en-4,6-diyn-3-yl acetate; (3R,8R)-8-hydroxyhexadeca-1-en-4,6-diyn-3-yl acetate; (3R,8S)-8-hydroxyhexadeca-1-en-4,6-diyn-3-yl acetate; (3S,8R)-8-hydroxypentadeca-1-en-4,6-diyn-3-yl acetate; (3S,8S)-8-hydroxypentadeca-1-en-4,6-diyn-3-yl acetate; (3R,8R)-8-hydroxypentadeca-1-en-4,6-diyn-3-yl acetate; (3R,8S)-8-hydroxypentadeca-1-en-4,6-diyn-3-yl acetate; (3S,8R)-8-hydroxytetradeca-1-en-4,6-diyn-3-yl acetate; (3S,8S)-8-hydroxytetradeca-1-en-4,6-diyn-3-yl acetate; (3R,8R)-8-hydroxytetradeca-1-en-4,6-diyn-3-yl acetate; (3R,8S)-8-hydroxytetradeca-1-en-4,6-diyn-3-yl acetate; (3S,8R)-8-hydroxytrideca-1-en-4,6-diyn-3-yl acetate; (3S,8S)-8-hydroxytrideca-1-en-4,6-diyn-3-yl acetate; (3R,8R)-8-hydroxytrideca-1-en-4,6-diyn-3-yl acetate; (3R,8S)-8-hydroxytrideca-1-en-4,6-diyn-3-yl acetate; (3S,8R)-8-hydroxyundeca-1-en-4,6-diyn-3-yl acetate; 8-hydroxy-10-phenyldeca-1-en-4,6-diyn-3-yl acetate; 8-hydroxypentacosa-1-en-4,6-diyn-3-yl acetate; 8-hydroxynonacosa-1-en-4,6-diyn-3-yl acetate; (3S,8R)-heptadeca-1-en-4,6-diyne-3,8-diyl diacetate; (3S,8S)-heptadeca-1-en-4,6-diyne-3,8-diyl diacetate; (3R,8R)-heptadeca-1-en-4,6-diyne-3,8-diyl diacetate; (3R,8S)-heptadeca-1-en-4,6-diyne-3,8-diyl diacetate; (3S,8R)-3-methoxyheptadeca-1-en-4,6-diyn-8-ol; (3S,8S)-3-methoxyheptadeca-1-en-4,6-diyn-8-ol; (3R,8R)-3-methoxyheptadeca-1-en-4,6-diyn-8-ol; (3R,8S)-3-methoxyheptadeca-1-en-4,6-diyn-8-ol; 8-((methylsulfonyl)oxy)heptadeca-1-en-4,6-diyn-3-yl acetate; 8-(tosyloxy)heptadeca-1-en-4,6-diyn-3-yl acetate; 8-acetamidoheptadeca-1-en-4,6-diyn-3-yl acetate; 8-(methylsulfonamido)heptadeca-1-en-4,6-diyn-3-yl acetate; 8-phenoxyheptadeca-1-en-4,6-diyn-3-yl acetate; 8-(acetylthio)heptadeca-1-en-4,6-diyn-3-yl acetate; 8-bromoheptadeca-1-en-4,6-diyn-3-yl acetate; (3S,8R)-8-(4-chlorobenzamido)heptadeca-1-en-4,6-diyn-3-yl acetate; (3S,8R)-8-(3,4-dichlorobenzamido)heptadeca-1-en-4,6-diyn-3-yl acetate; (3S,8R)-8-(4-methylbenzamido)heptadeca-1-en-4,6-diyn-3-yl acetate; (3S,8R)-8-(4-methoxybenzamido)heptadeca-1-en-4,6-diyn-3-yl acetate; (3S,8R)-8-hydroxyundeca-1-en-4,6-diyn-3-yl acetate; (3S,8R)-8-hydroxytricosa-1-en-4,6-diyn-3-yl acetate; (3S,8R)-8-aminoheptadeca-1-en-4,6-diyn-3-yl acetate; (3S,8R)-8-(methylsulfonamido)heptadeca-1-en-4,6-diyn-3-yl acetate; (3S,8R)-8-benzamidoheptadeca-1-en-4,6-diyn-3-yl acetate; (3S,8R)-8-(butylamino)heptadeca-1-en-4,6-diyn-3-yl acetate; (3S,8R)-8-((methylsulfonyl)oxy)heptadeca-1-en-4,6-diyn-3-yl acetate; (3S,8R)-heptadeca-1-en-4,6-diyne-3,8-diol; (3S,8S)-heptadeca-1-en-4,6-diyne-3,8-diol; (3R,8R)-heptadeca-1-en-4,6-diyne-3,8-diol; (3R,8S)-heptadeca-1-en-4,6-diyne-3,8-diol; (3S,8R)-8-aminoheptadeca-1-en-4,6-diyn-3-ol; (3S,8R)-8-(butylamino)heptadeca-1-en-4,6-diyn-3-ol; N-((3S,8R)-3-hydroxyheptadeca-1-en-4,6-diyn-8-yl)benzamide; ethyl((3S,8R)-3-hydroxyheptadeca-1-en-4,6-diyn-8-yl)carbamate; (3S,8R)-8-methoxyheptadeca-1-en-4,6-diyn-3-ol; N-((3S,8R)-3-hydroxyheptadeca-1-en-4,6-diyn-8-yl)acetamide; (3S,8R)-3-hydroxyheptadeca-1-en-4,6-diyn-8-yl acetate; (3S,8R)-8-(5-methylisoxazole-3-carboxamido)heptadeca-1-en-4,6-diyn-3-yl acetate; (3S,8R)-8-(picolinamido)heptadeca-1-en-4,6-diyn-3-yl acetate; (3S,8R)-8-(pyrazine-2-carboxamido)heptadeca-1-en-4,6-diyn-3-yl acetate; (3S,8R)-8-(2-chlorobenzamido)heptadeca-1-en-4,6-diyn-3-yl acetate; (3S,8R)-8-(3-bromobenzamido)heptadeca-1-en-4,6-diyn-3-yl acetate; (3S,8R)-8-(3-methylbenzamido)heptadeca-1-en-4,6-diyn-3-yl acetate; (3S,8R)-8-(4-fluorobenz amido)heptadeca-1-en-4,6-diyn-3-yl acetate; (3S,8R)-8-(3-(trifluoromethyl)benzamido)heptadeca-1-en-4,6-diyn-3-yl acetate; (3S,8R)-8-(furan-2-carboxamido)heptadeca-1-en-4,6-diyn-3-yl acetate; (3S,8R)-8-(3,5-dichlorobenzamido)heptadeca-1-en-4,6-diyn-3-yl acetate; (3S,8R)-8-isothiocyanatoheptadeca-1-en-4,6-diyn-3-yl acetate; (3S,8R)-8-(4-(benzyloxy)butanamido)heptadeca-1-en-4,6-diyn-3-yl acetate; (3S,8R)-8-((cyclopropylmethyl)amino)heptadeca-1-en-4,6-diyn-3-ol; (1S,6R)-6-(butylamino)-1-cyclopropylpentadeca-2,4-diyn-1-ol; (4S,9R,E)-9-(butylamino)octadeca-2-en-5,7-diyn-4-ol; (1S,6R)-6-(butylamino)-1-phenylpentadeca-2,4-diyn-1-ol; (1S,6R)-6-(butylamino)-1-(4-ethoxyphenyl)pentadeca-2,4-diyn-1-ol; (3S,8R)-8-(ethylamino)heptadeca-1-en-4,6-diyn-3-ol; or (3S,8R)-8-benzamido-11-cyclohexylundeca-1-en-4,6-diyn-3-yl acetate. In another embodiment, the compound is combined with a pharmaceutically acceptable excipient.

In a second aspect, the invention provides a method of treating a patient, such as a human, suffering from, or at risk of acquiring or developing, a protozoal or microbial infection or cancer by administering to the patient a therapeutically effective amount of a compound presented and defined by the following formula

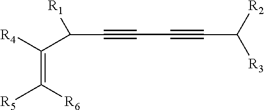

or a salt, ester or prodrug thereof, wherein
R₁ and R₂ vary independently and are selected from the group consisting of a hydrogen, halogen, hydroxyl, acetoxy, thiol, cyanide, azide, chlorosuccinimide, thiocyanate, amine, NHR₇, NR₇R₈, NR₇(C═O)R₈, C(═O)R₇, C(═O)NR₇R₈, NO₂, NH(SO₂)R₇, S(C═O)R₇, SO₂(N)R₇R₈, CO₂R₇, OR₇, OSO₂R₇, NR₇CO₂R₈, OC(═O)R₇, OC(═O)NR₇R₈, piperadino, pyrrolidino, piperazino, azetidino, morpholino, thiomorpholino, (C₁-C₂₀)alkyl, aryl(C₁-C₂₀)alkyl, and aryl, wherein any alkyl or aryl may be optionally substituted with one or more groups selected from R₂, and any alkyl group may optionally include one or more double or triple bonds within its carbon chain; and
R₃, R₄, R₅, R₆, R₇, and R₈ vary independently and are selected from the group consisting of a hydrogen, halogen, (C₁-C₂₀)alkyl, aryl(C₁-C₂₀)alkyl, and aryl, wherein any alkyl or aryl may be optionally substituted with one or more groups selected from R₂, and any alkyl or aryl alkyl group may optionally include one or more double or triple bonds within its carbon chain.

In one embodiment, the compound is (3S,8R)-8-hydroxyheptadeca-1-en-4,6-diyn-3-yl acetate; (3R,8R)-8-hydroxyheptadeca-1-en-4,6-diyn-3-yl acetate; (3R,8S)-8-hydroxyheptadeca-1-en-4,6-diyn-3-yl acetate; (3S,8R)-8-hydroxyhexadeca-1-en-4,6-diyn-3-yl acetate; (3S,8S)-8-hydroxyhexadeca-1-en-4,6-diyn-3-yl acetate; (3R,8R)-8-hydroxyhexadeca-1-en-4,6-diyn-3-yl acetate; (3R,8S)-8-hydroxyhexadeca-1-en-4,6-diyn-3-yl acetate; (3S,8R)-8-hydroxypentadeca-1-en-4,6-diyn-3-yl acetate; (3S,8S)-8-hydroxypentadeca-1-en-4,6-diyn-3-yl acetate; (3R,8R)-8-hydroxypentadeca-1-en-4,6-diyn-3-yl acetate; (3R,8S)-8-hydroxypentadeca-1-en-4,6-diyn-3-yl acetate; (3S,8R)-8-hydroxytetradeca-1-en-4,6-diyn-3-yl acetate; (3S,8S)-8-hydroxytetradeca-1-en-4,6-diyn-3-yl acetate; (3R,8R)-8-hydroxytetradeca-1-en-4,6-diyn-3-yl acetate; (3R,8S)-8-hydroxytetradeca-1-en-4,6-diyn-3-yl acetate; (3S,8R)-8-hydroxytrideca-1-en-4,6-diyn-3-yl acetate; (3S,8S)-8-hydroxytrideca-1-en-4,6-diyn-3-yl acetate; (3R,8R)-8-hydroxytrideca-1-en-4,6-diyn-3-yl acetate; (3R,8S)-8-hydroxytrideca-1-en-4,6-diyn-3-yl acetate; (3S,8R)-8-hydroxyundeca-1-en-4,6-diyn-3-yl acetate; 8-hydroxy-10-phenyldeca-1-en-4,6-diyn-3-yl acetate; 8-hydroxypentacosa-1-en-4,6-diyn-3-yl acetate; 8-hydroxynonacosa-1-en-4,6-diyn-3-yl acetate; (3S,8R)-heptadeca-1-en-4,6-diyne-3,8-diyl diacetate; (3S,8S)-heptadeca-1-en-4,6-diyne-3,8-diyl diacetate; (3R,8R)-heptadeca-1-en-4,6-diyne-3,8-diyl diacetate; (3R,8S)-heptadeca-1-en-4,6-diyne-3,8-diyl diacetate; (3S,8R)-3-methoxyheptadeca-1-en-4,6-diyn-8-ol; (3S,8S)-3-methoxyheptadeca-1-en-4,6-diyn-8-ol; (3R,8R)-3-methoxyheptadeca-1-en-4,6-diyn-8-ol; (3R,8S)-3-methoxyheptadeca-1-en-4,6-diyn-8-ol; 8-((methylsulfonyl)oxy)heptadeca-1-en-4,6-diyn-3-yl acetate; 8-(tosyloxy)heptadeca-1-en-4,6-diyn-3-yl acetate; 8-acetamidoheptadeca-1-en-4,6-diyn-3-yl acetate; 8-(methylsulfonamido)heptadeca-1-en-4,6-diyn-3-yl acetate; 8-phenoxyheptadeca-1-en-4,6-diyn-3-yl acetate; 8-(acetylthio)heptadeca-1-en-4,6-diyn-3-yl acetate; 8-bromoheptadeca-1-en-4,6-diyn-3-yl acetate; (3S,8R)-8-(4-chlorobenzamido)heptadeca-1-en-4,6-diyn-3-yl acetate; (3S,8R)-8-(3,4-dichlorobenzamido)heptadeca-1-en-4,6-diyn-3-yl acetate; (3S,8R)-8-(4-methylbenzamido)heptadeca-1-en-4,6-diyn-3-yl acetate; (3S,8R)-8-(4-methoxybenzamido)heptadeca-1-en-4,6-diyn-3-yl acetate; (3S,8R)-8-hydroxyundeca-1-en-4,6-diyn-3-yl acetate; (3S,8R)-8-hydroxytricosa-1-en-4,6-diyn-3-yl acetate; (3S,8R)-8-aminoheptadeca-1-en-4,6-diyn-3-yl acetate; (3S,8R)-8-(methylsulfonamido)heptadeca-1-en-4,6-diyn-3-yl acetate; (3S,8R)-8-benzamidoheptadeca-1-en-4,6-diyn-3-yl acetate; (3S,8R)-8-(butylamino)heptadeca-1-en-4,6-diyn-3-yl acetate; (3S,8R)-8-((methylsulfonyl)oxy)heptadeca-1-en-4,6-diyn-3-yl acetate; (3S,8R)-heptadeca-1-en-4,6-diyne-3,8-diol; (3S,8S)-heptadeca-1-en-4,6-diyne-3,8-diol; (3R,8R)-heptadeca-1-en-4,6-diyne-3,8-diol; (3R,8S)-heptadeca-1-en-4,6-diyne-3,8-diol; (3S,8R)-8-aminoheptadeca-1-en-4,6-diyn-3-ol; (3S,8R)-8-(butylamino)heptadeca-1-en-4,6-diyn-3-ol; N-((3S,8R)-3-hydroxyheptadeca-1-en-4,6-diyn-8-yl)benzamide; ethyl((3S,8R)-3-hydroxyheptadeca-1-en-4,6-diyn-8-yl)carbamate; (3S,8R)-8-methoxyheptadeca-1-en-4,6-diyn-3-ol; N-((3S,8R)-3-hydroxyheptadeca-1-en-4,6-diyn-8-yl)acetamide; (3S,8R)-3-hydroxyheptadeca-1-en-4,6-diyn-8-yl acetate; (3S,8R)-8-(5-methylisoxazole-3-carboxamido)heptadeca-1-en-4,6-diyn-3-yl acetate; (3S,8R)-8-(picolinamido)heptadeca-1-en-4,6-diyn-3-yl acetate; (3S,8R)-8-(pyrazine-2-carboxamido)heptadeca-1-en-4,6-diyn-3-yl acetate; (3S,8R)-8-(2-chlorobenzamido)heptadeca-1-en-4,6-diyn-3-yl acetate; (3S,8R)-8-(3-bromobenzamido)heptadeca-1-en-4,6-diyn-3-yl acetate; (3S,8R)-8-(3-methylbenzamido)heptadeca-1-en-4,6-diyn-3-yl acetate; (3S,8R)-8-(4-fluorobenz amido)heptadeca-1-en-4,6-diyn-3-yl acetate; (3S,8R)-8-(3-(trifluoromethyl)benzamido)heptadeca-1-en-4,6-diyn-3-yl acetate; (3S,8R)-8-(furan-2-carboxamido)heptadeca-1-en-4,6-diyn-3-yl acetate; (3S,8R)-8-(3,5-dichlorobenzamido)heptadeca-1-en-4,6-diyn-3-yl acetate; (3S,8R)-8-isothiocyanatoheptadeca-1-en-4,6-diyn-3-yl acetate; (3S,8R)-8-(4-(benzyloxy)butanamido)heptadeca-1-en-4,6-diyn-3-yl acetate; (3S,8R)-8-((cyclopropylmethyl)amino)heptadeca-1-en-4,6-diyn-3-ol; (1S,6R)-6-(butylamino)-1-cyclopropylpentadeca-2,4-diyn-1-ol; (4S,9R,E)-9-(butylamino)octadeca-2-en-5,7-diyn-4-ol; (1S,6R)-6-(butylamino)-1-phenylpentadeca-2,4-diyn-1-ol; (1S,6R)-6-(butylamino)-1-(4-ethoxyphenyl)pentadeca-2,4-diyn-1-ol; (3S,8R)-8-(ethylamino)heptadeca-1-en-4,6-diyn-3-ol; or (3S,8R)-8-benzamido-11-cyclohexylundeca-1-en-4,6-diyn-3-yl acetate. In another embodiment, the compound is in combination with a pharmaceutically acceptable excipient. In further embodiments, the compounds of the invention are used to treat protozoal infections caused by Leishmania, Plasmodium, or Trypanosoma or microbial infections is caused by S. aureus, E. faecalis, S. pyogenes, C. glabrata, B. subtilis, P. aeruginosa, B. antracis, or C. albicans. In another embodiment, the compounds of the invention are used to treat a cancer such as squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, breast cancer, colon cancer, colorectal cancer, endometrial carcinoma, salivary gland carcinoma, kidney cancer, renal cancer, prostate cancer, vulval cancer, thyroid cancer, hepatic carcinoma, or head and neck cancer.

In a third aspect, the invention provides a kit useful for treating a patient, such as a human, suffering from, or at risk of acquiring or developing, a protozoal or microbial infection or cancer by administering to the patient a therapeutically effective amount of a compound presented and defined by the following formula

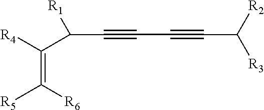

or a salt, ester or prodrug thereof, wherein
R₁ and R₂ vary independently and are selected from the group consisting of a hydrogen, halogen, hydroxyl, acetoxy, thiol, cyanide, azide, chlorosuccinimide, thiocyanate, amine, NHR₇, NR₇R₈, NR₇(C═O)R₈, C(═O)R₇, C(═O)NR₇R₈, NO₂, NH(SO₂)R₇, S(C═O)R₇, SO₂(N)R₇R₈, CO₂R₇, OR₇, OSO₂R₇, NR₇CO₂R₈, OC(═O)R₇, OC(═O)NR₇R₈, piperadino, pyrrolidino, piperazino, azetidino, morpholino, thiomorpholino, (C₁-C₂₀)alkyl, aryl(C₁-C₂₀)alkyl, and aryl, wherein any alkyl or aryl may be optionally substituted with one or more groups selected from R₂, and any alkyl group may optionally include one or more double or triple bonds within its carbon chain; and
R₃, R₄, R₅, R₆, R₇, and R₈ vary independently and are selected from the group consisting of a hydrogen, halogen, (C₁-C₂₀)alkyl, aryl(C₁-C₂₀)alkyl, and aryl, wherein any alkyl or aryl may be optionally substituted with one or more groups selected from R₂, and any alkyl or aryl alkyl group may optionally include one or more double or triple bonds within its carbon chain.

In one embodiment, the compound is in combination with a pharmaceutically acceptable excipient. In another embodiment, the compound is (3S,8R)-8-hydroxyheptadeca-1-en-4,6-diyn-3-yl acetate; (3R,8R)-8-hydroxyheptadeca-1-en-4,6-diyn-3-yl acetate; (3R,8S)-8-hydroxyheptadeca-1-en-4,6-diyn-3-yl acetate; (3S,8R)-8-hydroxyhexadeca-1-en-4,6-diyn-3-yl acetate; (3S,8S)-8-hydroxyhexadeca-1-en-4,6-diyn-3-yl acetate; (3R,8R)-8-hydroxyhexadeca-1-en-4,6-diyn-3-yl acetate; (3R,8S)-8-hydroxyhexadeca-1-en-4,6-diyn-3-yl acetate; (3S,8R)-8-hydroxypentadeca-1-en-4,6-diyn-3-yl acetate; (3S,8S)-8-hydroxypentadeca-1-en-4,6-diyn-3-yl acetate; (3R,8R)-8-hydroxypentadeca-1-en-4,6-diyn-3-yl acetate; (3R,8S)-8-hydroxypentadeca-1-en-4,6-diyn-3-yl acetate; (3S,8R)-8-hydroxytetradeca-1-en-4,6-diyn-3-yl acetate; (3S,8S)-8-hydroxytetradeca-1-en-4,6-diyn-3-yl acetate; (3R,8R)-8-hydroxytetradeca-1-en-4,6-diyn-3-yl acetate; (3R,8S)-8-hydroxytetradeca-1-en-4,6-diyn-3-yl acetate; (3S,8R)-8-hydroxytrideca-1-en-4,6-diyn-3-yl acetate; (3S,8S)-8-hydroxytrideca-1-en-4,6-diyn-3-yl acetate; (3R,8R)-8-hydroxytrideca-1-en-4,6-diyn-3-yl acetate; (3R,8S)-8-hydroxytrideca-1-en-4,6-diyn-3-yl acetate; (3S,8R)-8-hydroxyundeca-1-en-4,6-diyn-3-yl acetate; 8-hydroxy-10-phenyldeca-1-en-4,6-diyn-3-yl acetate; 8-hydroxypentacosa-1-en-4,6-diyn-3-yl acetate; 8-hydroxynonacosa-1-en-4,6-diyn-3-yl acetate; (3S,8R)-heptadeca-1-en-4,6-diyne-3,8-diyl diacetate; (3S,8S)-heptadeca-1-en-4,6-diyne-3,8-diyl diacetate; (3R,8R)-heptadeca-1-en-4,6-diyne-3,8-diyl diacetate; (3R,8S)-heptadeca-1-en-4,6-diyne-3,8-diyl diacetate; (3S,8R)-3-methoxyheptadeca-1-en-4,6-diyn-8-ol; (3S,8S)-3-methoxyheptadeca-1-en-4,6-diyn-8-ol; (3R,8R)-3-methoxyheptadeca-1-en-4,6-diyn-8-ol; (3R,8S)-3-methoxyheptadeca-1-en-4,6-diyn-8-ol; 8-((methylsulfonyl)oxy)heptadeca-1-en-4,6-diyn-3-yl acetate; 8-(tosyloxy)heptadeca-1-en-4,6-diyn-3-yl acetate; 8-acetamidoheptadeca-1-en-4,6-diyn-3-yl acetate; 8-(methylsulfonamido)heptadeca-1-en-4,6-diyn-3-yl acetate; 8-phenoxyheptadeca-1-en-4,6-diyn-3-yl acetate; 8-(acetylthio)heptadeca-1-en-4,6-diyn-3-yl acetate; 8-bromoheptadeca-1-en-4,6-diyn-3-yl acetate; (3S,8R)-8-(4-chlorobenzamido)heptadeca-1-en-4,6-diyn-3-yl acetate; (3S,8R)-8-(3,4-dichlorobenzamido)heptadeca-1-en-4,6-diyn-3-yl acetate; (3S,8R)-8-(4-methylbenzamido)heptadeca-1-en-4,6-diyn-3-yl acetate; (3S,8R)-8-(4-methoxybenzamido)heptadeca-1-en-4,6-diyn-3-yl acetate; (3S,8R)-8-hydroxyundeca-1-en-4,6-diyn-3-yl acetate; (3S,8R)-8-hydroxytricosa-1-en-4,6-diyn-3-yl acetate; (3S,8R)-8-aminoheptadeca-1-en-4,6-diyn-3-yl acetate; (3S,8R)-8-(methylsulfonamido)heptadeca-1-en-4,6-diyn-3-yl acetate; (3S,8R)-8-benzamidoheptadeca-1-en-4,6-diyn-3-yl acetate; (3S,8R)-8-(butylamino)heptadeca-1-en-4,6-diyn-3-yl acetate; (3S,8R)-8-((methylsulfonyl)oxy)heptadeca-1-en-4,6-diyn-3-yl acetate; (3S,8R)-heptadeca-1-en-4,6-diyne-3,8-diol; (3S,8S)-heptadeca-1-en-4,6-diyne-3,8-diol; (3R,8R)-heptadeca-1-en-4,6-diyne-3,8-diol; (3R,8S)-heptadeca-1-en-4,6-diyne-3,8-diol; (3S,8R)-8-aminoheptadeca-1-en-4,6-diyn-3-ol; (3S,8R)-8-(butylamino)heptadeca-1-en-4,6-diyn-3-ol; N-((3S,8R)-3-hydroxyheptadeca-1-en-4,6-diyn-8-yl)benzamide; ethyl((3S,8R)-3-hydroxyheptadeca-1-en-4,6-diyn-8-yl)carbamate; (3S,8R)-8-methoxyheptadeca-1-en-4,6-diyn-3-ol; N-((3S,8R)-3-hydroxyheptadeca-1-en-4,6-diyn-8-yl)acetamide; (3S,8R)-3-hydroxyheptadeca-1-en-4,6-diyn-8-yl acetate; (3S,8R)-8-(5-methylisoxazole-3-carboxamido)heptadeca-1-en-4,6-diyn-3-yl acetate; (3S,8R)-8-(picolinamido)heptadeca-1-en-4,6-diyn-3-yl acetate; (3S,8R)-8-(pyrazine-2-carboxamido)heptadeca-1-en-4,6-diyn-3-yl acetate; (3S,8R)-8-(2-chlorobenzamido)heptadeca-1-en-4,6-diyn-3-yl acetate; (3S,8R)-8-(3-bromobenzamido)heptadeca-1-en-4,6-diyn-3-yl acetate; (3S,8R)-8-(3-methylbenzamido)heptadeca-1-en-4,6-diyn-3-yl acetate; (3S,8R)-8-(4-fluorobenzamido)heptadeca-1-en-4,6-diyn-3-yl acetate; (3S,8R)-8-(3-(trifluoromethyl)benzamido)heptadeca-1-en-4,6-diyn-3-yl acetate; (3S,8R)-8-(furan-2-carboxamido)heptadeca-1-en-4,6-diyn-3-yl acetate; (3S,8R)-8-(3,5-dichlorobenzamido)heptadeca-1-en-4,6-diyn-3-yl acetate; (3S,8R)-8-isothiocyanatoheptadeca-1-en-4,6-diyn-3-yl acetate; (3S,8R)-8-(4-(benzyloxy)butanamido)heptadeca-1-en-4,6-diyn-3-yl acetate; (3S,8R)-8-((cyclopropylmethyl)amino)heptadeca-1-en-4,6-diyn-3-ol; (1S,6R)-6-(butylamino)-1-cyclopropylpentadeca-2,4-diyn-1-ol; (4S,9R,E)-9-(butylamino)octadeca-2-en-5,7-diyn-4-ol; (1S,6R)-6-(butylamino)-1-phenylpentadeca-2,4-diyn-1-ol; (1S,6R)-6-(butylamino)-1-(4-ethoxyphenyl)pentadeca-2,4-diyn-1-ol; (3S,8R)-8-(ethylamino)heptadeca-1-en-4,6-diyn-3-ol; or (3S,8R)-8-benzamido-11-cyclohexylundeca-1-en-4,6-diyn-3-yl acetate. In another embodiment, the compound is in combination with a pharmaceutically acceptable excipient. In further embodiments, the compounds of the invention are used to treat protozoal infections caused by Leishmania, Plasmodium, or Trypanosoma or microbial infections is caused by S. aureus, E. faecalis, S. pyogenes, C. glabrata, B. subtilis, P. aeruginosa, B. antracis, or C. albicans. In another embodiment, the compounds of the invention are used to treat a cancer such as squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, breast cancer, colon cancer, colorectal cancer, endometrial carcinoma, salivary gland carcinoma, kidney cancer, renal cancer, prostate cancer, vulval cancer, thyroid cancer, hepatic carcinoma, and head and neck cancer.

In a fourth embodiment, the invention features methods of synthesizing a compounds presented and defined by the following formula

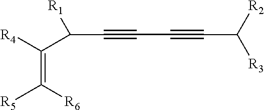

or a salt, ester or prodrug thereof, wherein
R₁ and R₂ vary independently and are selected from the group consisting of a hydrogen, halogen, hydroxyl, acetoxy, thiol, cyanide, azide, chlorosuccinimide, thiocyanate, amine, NHR₇, NR₇R₈, NR₇(C═O)R₈, C(═O)R₇, C(═O)NR₇R₈, NO₂, NH(SO₂)R₇, S(C═O)R₇, SO₂(N)R₇R₈, CO₂R₇, OR₇, OSO₂R₇, NR₇CO₂R₈, OC(═O)R₇, OC(═O)NR₇R₈, piperadino, pyrrolidino, piperazino, azetidino, morpholino, thiomorpholino, (C₁-C₂₀)alkyl, aryl(C₁-C₂₀)alkyl, and aryl, wherein any alkyl or aryl may be optionally substituted with one or more groups selected from R₂, and any alkyl group may optionally include one or more double or triple bonds within its carbon chain; and
R₃, R₄, R₅, R₆, R₇, and R₈ vary independently and are selected from the group consisting of a hydrogen, halogen, (C₁-C₂₀)alkyl, aryl(C₁-C₂₀)alkyl, and aryl, wherein any alkyl or aryl may be optionally substituted with one or more groups selected from R₂, and any alkyl or aryl alkyl group may optionally include one or more double or triple bonds within its carbon chain;
   such as (3S,8R)-8-hydroxyheptadeca-1-en-4,6-diyn-3-yl acetate (compound 1), by reacting, e.g., n-butylamine with Copper (I) chloride to yield a primary adduct, reacting the primary adduct with (R)-dodec-1-yn-3-ol to yield a secondary adduct, and reacting the secondary adduct with (S)-5-bromopent-1-en-4-yn-3-yl acetate to yield compound 1.

Further embodiments, features, and advantages of the present inventions, as well as the structure and operation of the various embodiments of the present invention, are described in detail below with reference to the accompanying drawings.

### BRIEF DESCRIPTION OF THE SEVERAL VIEWS OF THE DRAWINGS

The accompanying drawings, which are incorporated herein and form a part of the specification, illustrate one or more embodiments of the present invention and, together with the description, further serve to explain the principles of the invention and to enable a person skilled in the pertinent art to make and use the invention.

FIG. 1 is a list of exemplary compounds of the invention, their chemical structures, and chemical names.

FIG. 2 consists of two bar graphs that depict A. the total liver parasitemia (LDU) and B. the percent reduction in liver parasitemia in Balb/C mice infected with leishmaniasis following treatment with: control, vehicle, miltefisone, compound 47 (“DB-12”), compound 48 (“DB-13”), or compound 50 (“DB-14”).

FIG. 3 consists of two bar graphs that depict the anti-cancer activity (i.e., growth inhibition or cell killing) of A. single-dose (10 μM) B. dose-response (5 different doses) of compound 1 against the NCI-60 human tumor cell line panel.

FIG. 4 consists of two bar graphs that depict the anti-cancer activity (i.e., growth inhibition or cell killing) of A. single-dose (10 μM) or B. dose-response (5 different doses) of compound 1 against the NCI-60 human tumor cell line panel.

FIG. 5 consists of a bar graph that depicts the anti-cancer activity (i.e., growth inhibition or cell killing) of a single-dose (10 μM) of compound 44 against the NCI-60 human tumor cell line panel.

FIG. 6 consists of a bar graph that depicts the anti-cancer activity (i.e., growth inhibition or cell killing) of a single-dose (10 μM) of compound 45 against the NCI-60 human tumor cell line panel.

FIG. 7 consists of two bar graphs that depict the anti-cancer activity (i.e., growth inhibition or cell killing) of A. single-dose (10 μM) or B. dose-response (5 different doses) of compound 46 against the NCI-60 human tumor cell line panel.

FIG. 8 consists of a bar graph that depicts the anti-cancer activity (i.e., growth inhibition or cell killing) of a single-dose (10 μM) of compound 47 against the NCI-60 human tumor cell line panel.

FIG. 9 consists of a bar graph that depicts the anti-cancer activity (i.e., growth inhibition or cell killing) of a single-dose (10 μM) of compound 48 against the NCI-60 human tumor cell line panel.

FIG. 10 consists of a bar graph that depicts the anti-cancer activity (i.e., growth inhibition or cell killing) of a single-dose (10 μM) of compound 49 against the NCI-60 human tumor cell line panel.

FIG. 11 consists of a bar graph that depicts the anti-cancer activity (i.e., growth inhibition or cell killing) of a single-dose (10 μM) of compound 50 against the NCI-60 human tumor cell line panel.

FIG. 12 consists of two bar graphs that depict the anti-cancer activity (i.e., growth inhibition or cell killing) of A. single-dose (10 μM) or B. dose-response (5 different doses) of compound 61 against the NCI-60 human tumor cell line panel.

FIG. 13 consists of two bar graphs that depict the anti-cancer activity (i.e., growth inhibition or cell killing) of A. single-dose (10 μM) or B. dose-response (5 different doses) of compound 62 against the NCI-60 human tumor cell line panel.

FIG. 14 consists of two bar graphs that depict the anti-cancer activity (i.e., growth inhibition or cell killing) of A. single-dose (10 μM) or B. dose-response (5 different doses) of compound 63 against the NCI-60 human tumor cell line panel.

FIG. 15 consists of two bar graphs that depict the anti-cancer activity (i.e., growth inhibition or cell killing) of A. single-dose (10 μM) or B. dose-response (5 different doses) of compound 64 against the NCI-60 human tumor cell line panel.

FIG. 16 consists of a bar graph that depicts the anti-cancer activity (i.e., growth inhibition or cell killing) of a single-dose (10 μM) of compound 65 against the NCI-60 human tumor cell line panel.

FIG. 17 consists of two bar graphs that depict the anti-cancer activity (i.e., growth inhibition or cell killing) of A. single-dose (10 μM) or B. dose-response (5 different doses) of compound 66 against the NCI-60 human tumor cell line panel.

FIG. 18 consists of two bar graphs that depict the anti-cancer activity (i.e., growth inhibition or cell killing) of A. single-dose (10 μM) or B. dose-response (5 different doses) of compound 67 against the NCI-60 human tumor cell line panel.

FIG. 19 consists of a bar graph that depicts the anti-cancer activity (i.e., growth inhibition or cell killing) of a single-dose (10 μM) of compound 68 against the NCI-60 human tumor cell line panel.

FIG. 20 consists of two bar graphs that depict the anti-cancer activity (i.e., growth inhibition or cell killing) of A. single-dose (10 μM) or B. dose-response (5 different doses) of compound 69 against the NCI-60 human tumor cell line panel.

FIG. 21 consists of a bar graph that depicts the anti-cancer activity (i.e., growth inhibition or cell killing) of a single-dose (10 μM) of compound 70 against the NCI-60 human tumor cell line panel.

FIG. 22 consists of a bar graph that depicts the anti-cancer activity (i.e., growth inhibition or cell killing) of a single-dose (10 μM) of compound 71 against the NCI-60 human tumor cell line panel.

FIG. 23 consists of two bar graphs that depict the anti-cancer activity (i.e., growth inhibition or cell killing) of A. single-dose (10 μM) or B. dose-response (5 different doses) of compound 72 against the NCI-60 human tumor cell line panel.

### DETAILED DESCRIPTION OF THE INVENTION

### I. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

As used herein, the singular form “a,” “an,” and “the” include plural references unless the context clearly dictates otherwise. For example, the term “a cell” includes a plurality of cells, including mixtures thereof. The term “a nucleic acid molecule” includes a plurality of nucleic acid molecules.

As used herein, the terms below have the meanings indicated.

The term “acyl” as used herein, alone or in combination, refers to a carbonyl attached to an alkenyl, alkyl, aryl, cycloalkyl, heteroaryl, heterocyclyl, or any other moiety where the atom attached to the carbonyl is carbon. An “acetyl” group refers to a —C(O)CH₃ group.

An “alkyl carbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl” as used herein, alone or in combination, refers to a straight-chain or branched-chain hydrocarbon group having one or more double bonds optionally substituted and containing from 2 to 20, preferably 2 to 6, carbon atoms. Alkenyl refers to a carbon-carbon double bond system attached at two or more positions such as ethenylene [(—CH═CH—),(—C::C—)]. Examples of alkenyl groups include ethenyl, propenyl, 2-methylpropenyl, 1,4-butadienyl and the like.

The term “alkoxy” as used herein, alone or in combination, refers to an alkyl ether group, optionally substituted wherein the term alkyl is as defined below. Examples of alkyl ether groups include methoxy, ethoxy, n-propoxy, isopropoxy, n-butoxy, iso-butoxy, sec-butoxy, tert-butoxy, and the like.

The term “alkyl” as used herein, alone or in combination, refers to a straight-chain or branched-chain alkyl group optionally substituted containing from 1 to 20 and including 20, preferably 1 to 10, and more preferably 1 to 6, carbon atoms. Alkyl groups may be optionally substituted as defined herein. Examples of alkyl groups include methyl, ethyl, n-propyl, isopropyl, n-butyl, isobutyl, sec-butyl, tert-butyl, pentyl, iso-amyl, hexyl, octyl, nonyl and the like.

The term “alkylamino” as used herein, alone or in combination, refers to an alkyl group optionally substituted attached to the parent molecular moiety through an amino group. Alkylamino groups may be mono- or dialkylated, forming groups such as, for example, N-methylamino, N-ethylamino, N,N-dimethylamino, N,N-ethylmethylamino and the like.

The term “alkylthio” as used herein, alone or in combination, refers to an alkyl thioether (R—S—) group wherein the term alkyl is as defined above and wherein the sulfur may be singly or doubly oxidized. Examples of alkyl thioether groups include methylthio, ethylthio, n-propylthio, isopropylthio, n-butylthio, iso-butylthio, sec-butylthio, tert-butylthio, methanesulfonyl, ethanesulfinyl, and the like.

The term “alkynyl” as used herein, alone or in combination, refers to a straight-chain or branched chain hydrocarbon group having one or more triple bonds and containing from 2 to 20, preferably from 2 to 6, more preferably from 2 to 4, carbon atoms. “Alkynyl” refers to a carbon-carbon triple bond attached at two positions such as ethynylene (—C:::C—, —C≡C—). Examples of alkynyl groups include ethynyl, propynyl, hydroxypropynyl, butyn-1-butyn-2-yl, pentyn-1-yl, 3-methylbutyn-1-yl, hexyn-2-yl, and the like.

The term “amido” as used herein, alone or in combination, refer to an amino group as described below attached to the parent molecular moiety through a carbonyl group, or vice versa.

The term “amino” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted.

The term “aryl” as used herein, alone or in combination, means a carbocyclic aromatic system containing one, two or three rings wherein such rings may be attached together in a pendent manner or may be fused optionally substituted with at least one halogen, an alkyl containing from 1 to 3 carbon atoms, an alkoxyl, an aryl group, a nitro function, a polyether group, a heteroaryl group, a benzoyl group, an alkyl ester group, a carboxylic acid, a hydroxyl optionally protected with an acetyl or benzoyl group, or an amino function optionally protected with an acetyl or benzoyl group or optionally substituted with at least one alkyl group containing from 1 to 12 carbon atoms.

The terms “arylalkyl” or “aralkyl” as used herein, alone or in combination, refers to an aryl group attached to the parent molecular moiety through an alkyl group.

The term “aryloxy” as used herein, alone or in combination, refers to an aryl group attached to the parent molecular moiety through an oxygen atom.

The term “polyether group” means a polyether group containing from 2 to 6 carbon atoms interrupted with at least one oxygen atom, such as methoxymethyl, ethoxymethyl or methoxyethoxymethyl groups or methoxyethyl.

The terms “benzo” and “benz” as used herein, alone or in combination, refer to the divalent group C₆H₄═ derived from benzene (e.g., a benzene group in a fused ring system). Examples include benzothiophene and benzimidazole.

The terms “cancer” refers to or describes the physiological condition in mammals that is typically characterized by unregulated cell growth. Examples of cancer include but are not limited to carcinoma, lymphoma, blastoma, sarcoma, and leukemia. More particular examples of such cancers include squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, breast cancer, colon cancer, colorectal cancer, endometrial carcinoma, salivary gland carcinoma, kidney cancer, renal cancer, prostate cancer, vulval cancer, thyroid cancer, hepatic carcinoma, and various types of head and neck cancer.

The terms “carbamate” and “carbamoyl” as used herein, alone or in combination, refers to an ester of carbanic acid (—NHCOO—) which may be attached to the parent molecular moiety from either the nitrogen or acid end, and which may be optionally substituted as defined herein.

The term “carbonyl” as used herein, when alone includes formyl [—C(O)H] and in combination is a —C(O)— group.

The term “carboxy” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano” as used herein, alone or in combination, refers to —CN.

The term “cycloalkyl” or, alternatively, “carbocycle”, as used herein, alone or in combination, refers to a saturated or partially saturated monocyclic, bicyclic or tricyclic alkyl group wherein each cyclic moiety contains from 3 to 12, preferably five to seven, carbon atom ring members and which may optionally be a benzo-fused ring system which is optionally substituted as defined herein. Examples of such cycloalkyl groups include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydronapthalene, octahydronapthalene as well as the multicyclic (multicentered) saturated or partially unsaturated type. The latter type of isomer is exemplified in general by, bicyclo[1.1.1]pentane, camphor, adamantane, and bicyclo[3.2.1]octane.

The term “ester” as used herein, alone or in combination, refers to a carboxy group bridging two moieties linked at carbon atoms.

The term “ether” as used herein, alone or in combination, refers to an oxygen atom bridging two moieties linked at carbon atoms.

The terms “halo” or “halogen” as used herein, alone or in combination, refers to fluorine, chlorine, bromine, or iodine.

The term “haloalkyl” as used herein, alone or in combination, refers to an alkyl group having the meaning as defined above wherein one or more hydrogens are replaced with a halogen. Specifically embraced are monohaloalkyl, dihaloalkyl and polyhaloalkyl groups. A monohaloalkyl group, for one example, may have an iodo, bronco, chloro or fluoro atom within the group. Dihalo and polyhaloalkyl groups may have two or more of the same halo atoms or a combination of different halo groups. Examples of haloalkyl groups include fluoromethyl, difluoromethyl, trifluoromethyl, chloromethyl, di chloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CHF—), difluoromethylene (—CF₂—), chloromethylene (—CHCl—) and the like.

The term “heteroalkyl” as used herein, alone or in combination, refers to a stable straight or branched chain, or cyclic hydrocarbon group, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed, at any interior position of the heteroalkyl group. Up to two heteroatoms may be consecutive, such as, for example, —CH₂—NH—OCH₃.

The term “heteroaryl” as used herein, alone or in combination, refers to 3 to 7 membered, preferably 5 to 7 membered, unsaturated heteromonocyclic rings, or fused polycyclic rings in which at least one of the fused rings is unsaturated, wherein at least one atom is selected from the group consisting of O, S, and N. The term also embraces fused polycyclic groups wherein heterocyclic groups are fused with aryl groups, wherein heteroaryl groups are fused with other heteroaryl groups, or wherein heteroaryl groups are fused with cycloalkyl groups. Examples of heteroaryl groups include pyrrolyl, pyrrolinyl pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocyclyl”, as used herein, alone or in combination, each refer to a saturated, partially unsaturated, or fully unsaturated monocyclic, bicyclic, or tricyclic heterocyclic group containing at least one, preferably 1 to 4, and, more preferably 1 to 2 heteroatoms as ring members, wherein each said heteroatom may be independently selected from the group consisting of nitrogen, oxygen, and sulfur, and wherein there are preferably 3 to 8 ring members in each ring, more preferably 3 to 7 ring members in each ring, and most preferably 5 to 6 ring members in each ring. “Heterocycloalkyl” and “heterocyclyl” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Heterocyclyl groups of the invention are exemplified by aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, morpholinyl, piperazinyl, pyrrolidinyl, tetrahydropyridinyl, piperidinyl, thiomorpholinyl, and the like. The heterocyclyl groups may be optionally substituted unless specifically prohibited.

The term “hydroxyl” as used herein, alone or in combination, refers to OH.

The phrase “in the main chain” refers to the longest contiguous or adjacent chain of carbon atoms starting at the point of attachment of a group to the compounds of this invention.

The phrase “linear chain of atoms” refers to the longest straight chain of atoms independently selected from carbon, nitrogen, oxygen and sulfur.

The term “lower” as used herein, alone or in combination, means containing from 1 to and including: 6 carbon atoms.

The terms “microbe” and “microbial” as used herein refer to any microorganism, including, but not limited to bacteria, fungi, yeast, viruses, archaea, and protists. Microbes can be unicellular or multicellular. As used herein, an “antimicrobial” is a chemical agent that is effective at killing microbes, inhibiting the proliferation of microbes, or reducing the morbidity or mortality associated with microbial infection.

The term “negatively-charged ion” as used herein, refers to any negatively-charged ion or molecule, either inorganic (e.g., Cl⁻, Br⁻, I⁻) or organic (e.g., TsO— (i.e., tosylate)).

The term “nitro” as used herein, alone or in combination, refers to —NO₂.

The term “perhaloalkyl” as used herein, alone or in combination, refers to an alkyl. group where all of the hydrogen atoms are replaced by halogen atoms.

The terms “protozoa” or “protozoal” as used herein refer to eukaryotic unicellular organisms, including, but not limited to flagellates, amoeboids, sporozoans, and ciliates. The compounds of the invention can be used to treat protozoal infections such as, e.g., Leishmania, Plasmodium, and Trypanosoma infections. As used herein, an “antiprotozoal” is a chemical agent that is effective at killing protozoa, inhibiting the proliferation of protozoa, or reducing the morbidity or mortality associated with protozoal infection.

Any definition herein may be used in combination with any other definition to describe a composite structural group. By convention, the trailing, element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, arylthio, lower alkylsulfinyl, lower alkylsulfonyl, arylsulfinyl, arylsulfonyl, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

Asymmetric centers exist in the compounds of the present invention. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and l-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

Optical isomers are compounds with the same molecular formula but differ in the way they rotate plane polarized light. There are two types of optical isomers. The first type of optical isomers are compounds that are mirror images of one another but cannot be superimposed on each other. These isomers are called “enantiomers.” The second type of optical isomers are molecules that are not mirror images but each molecule rotates plane polarized light and are considered optically-active. Such molecules are called “diastereoisomers.” Diasteroisomers differ not only in the way they rotate plane polarized light, but also their physical properties. The term “optical isomer” comprises more particularly the enantiomers and the diastereoisomers, in pure form or in the form of a mixture.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “imaging agent” as used herein refers to any moiety useful for the detection, tracing, or visualization of a compound of the invention when coupled thereto. Imaging agents include, e.g., an enzyme, a fluorescent label (e.g., fluorescein), a luminescent label, a bioluminescent label, a magnetic label, a metallic particle (e.g., a gold particle), a nanoparticle, an antibody or fragment thereof (e.g., a Fab, Fab′, or F(ab′)₂ molecule), and biotin. An imaging agent can be coupled to a compound of the invention by, for example, a covalent bond, ionic bond, van der Waals interaction or a hydrophobic bond. An imaging agent of the invention can be a radiolabel coupled to a compound of the invention, or a radioisotope incorporated into the chemical structure of a compound of the invention. Methods of detecting such imaging agents are well known to those having skill in the art.

The phrase “therapeutically effective” is intended to qualify the amount of active ingredients used in the treatment of a disease or disorder. This amount will achieve the goal of reducing or eliminating the disease or disorder.

The term “therapeutically acceptable” refers to those compounds (or salts, esters, prodrugs, tautomers, zwitterionic forms, etc. thereof) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. The term “patient” means all mammals including humans. Examples of patients include humans, cows, dogs, cats, goats, sheep, pigs, rabbits, and rodents (e.g., rats, mice, and guinea pigs).

The term “prodrug” refers to a compound that is made more active in vivo. Certain compounds of the present invention may also exist as prodrugs, as described in Hydrolysis in Drug and Prodrug Metabolism: Chemistry, Biochemistry, and Enzymology, Testa, Bernard and Wiley-VHCA, Zurich, Switzerland 2003. Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bio-available by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug is a compound that is administered as an ester (the “prodrug”), but then is metabolically hydrolyzed to the carboxylic acid, the active entity. Additional examples include peptidyl derivatives of a compound.

The compounds of the invention can exist as therapeutically acceptable salts. The present invention includes compounds listed above in the form of salts, in particular acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Stahl, P. Heinrich, Pharmaceutical Salts: Properties, Selection, and Use, Wiley-VCHA, Zurich, Switzerland (2002).

The term “therapeutically acceptable salt” as used herein, represents salts or zwitterionic forms of the compounds of the present invention which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, D-tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds of the compounds of the present invention and the like.

### II. Modes for Carrying Out the Invention

### A. Compounds with Antiprotozoal, Antimicrobial, and Anti-Cancer Properties

The present invention features compounds with antiprotozoal, antimicrobial, and anti-cancer activity, useful for the treatment of patients (e.g., humans) suffering from or at risk of developing protozoal or microbial infections or cancer. The compounds of the invention are represented by the following formula:

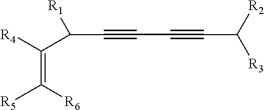

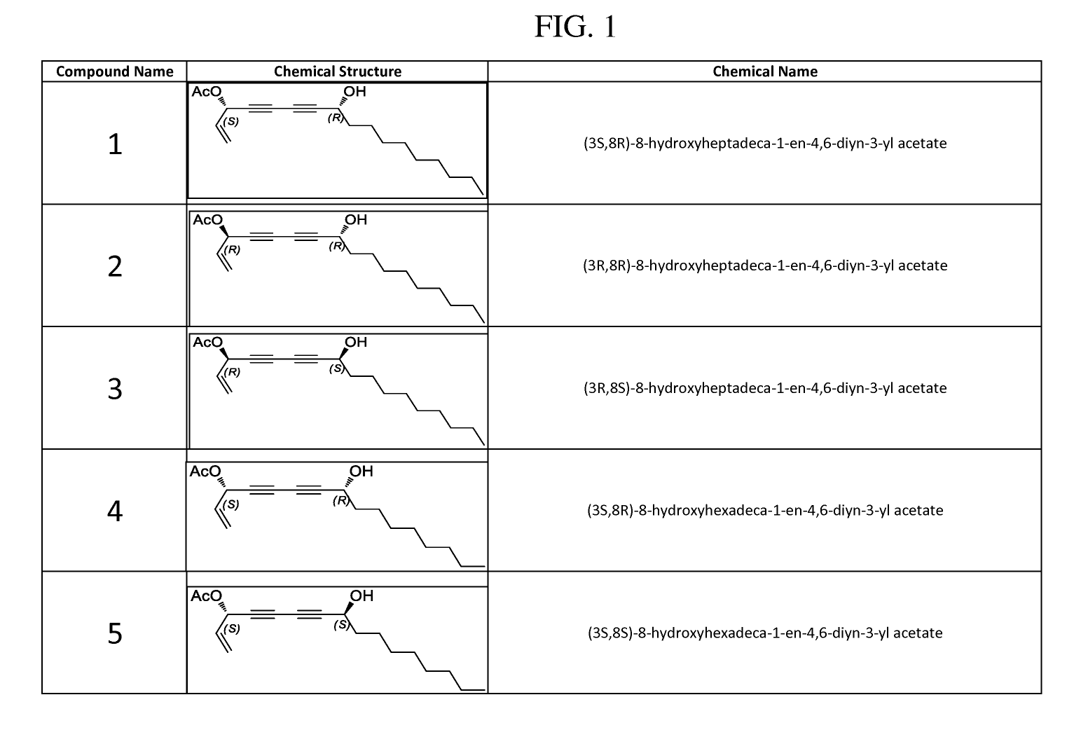

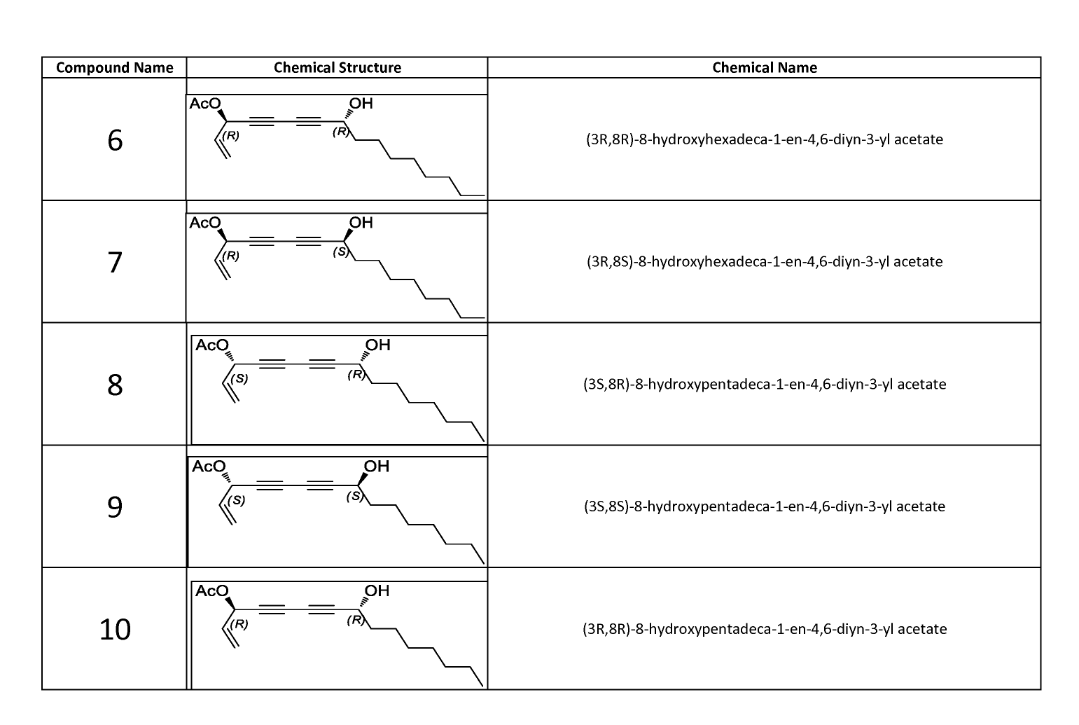

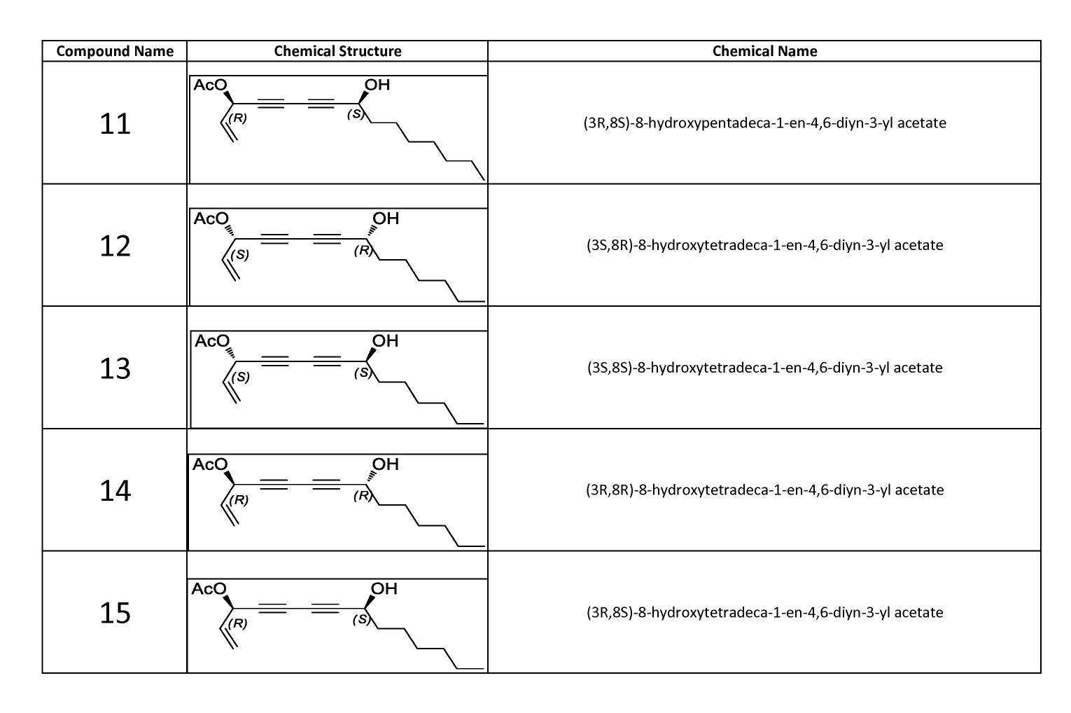

or a salt, ester or prodrug thereof, wherein R₁ and R₂ vary independently and are selected from the group consisting of a hydrogen, halogen, hydroxyl, acetoxy, thiol, cyanide, azide, chlorosuccinimide, thiocyanate, amine, NHR₇, NR₇R₈, NR₇(C═O)R₈, C(═O)R₇, C(═O)NR₇R₈, NO₂, NH(SO₂)R₇, S(C═O)R₇, SO₂(N)R₇R₈, CO₂R₇, OR₇, OSO₂R₇, NR₇CO₂R₈, OC(═O)R₇, OC(═O)NR₇R₈, piperadino, pyrrolidino, piperazino, azetidino, morpholino, thiomorpholino, (C₁-C₂₀)alkyl, aryl(C₁-C₂₀)alkyl, and aryl, wherein any alkyl or aryl may be optionally substituted with one or more groups selected from R₂, and any alkyl group may optionally include one or more double or triple bonds within its carbon chain; and wherein R₃, R₄, R₅, R₆, R₇, and R₈ vary independently and are selected from the group consisting of a hydrogen, halogen, (C₁-C₂₀)alkyl, aryl(C₁-C₂₀)alkyl, and aryl, wherein any alkyl or aryl may be optionally substituted with one or more groups selected from R₂, and any alkyl or aryl alkyl group may optionally include one or more double or triple bonds within its carbon chain. In one embodiment of the invention, the compound is (3S,8R)-8-hydroxyheptadeca-1-en-4,6-diyn-3-yl acetate, (3R,8R)-8-hydroxyheptadeca-1-en-4,6-diyn-3-yl acetate, (3R,8S)-8-hydroxyheptadeca-1-en-4,6-diyn-3-yl acetate, (3S,8R)-8-hydroxyhexadeca-1-en-4,6-diyn-3-yl acetate, (3S,8S)-8-hydroxyhexadeca-1-en-4,6-diyn-3-yl acetate, (3R,8R)-8-hydroxyhexadeca-1-en-4,6-diyn-3-yl acetate, (3R,8S)-8-hydroxyhexadeca-1-en-4,6-diyn-3-yl acetate, (3S,8R)-8-hydroxypentadeca-1-en-4,6-diyn-3-yl acetate, (3S,8S)-8-hydroxypentadeca-1-en-4,6-diyn-3-yl acetate, (3R,8R)-8-hydroxypentadeca-1-en-4,6-diyn-3-yl acetate, (3R,8S)-8-hydroxypentadeca-1-en-4,6-diyn-3-yl acetate, (3S,8R)-8-hydroxytetradeca-1-en-4,6-diyn-3-yl acetate, (3S,8S)-8-hydroxytetradeca-1-en-4,6-diyn-3-yl acetate, (3R,8R)-8-hydroxytetradeca-1-en-4,6-diyn-3-yl acetate, (3R,8S)-8-hydroxytetradeca-1-en-4,6-diyn-3-yl acetate, (3S,8R)-8-hydroxytrideca-1-en-4,6-diyn-3-yl acetate, (3S,8 5)-8-hydroxytrideca-1-en-4,6-diyn-3-yl acetate, (3R,8R)-8-hydroxytrideca-1-en-4,6-diyn-3-yl acetate, (3R,8S)-8-hydroxytrideca-1-en-4,6-diyn-3-yl acetate, (3S,8R)-8-hydroxyundeca-1-en-4,6-diyn-3-yl acetate, 8-hydroxy-10-phenyldeca-1-en-4,6-diyn-3-yl acetate, 8-hydroxypentacosa-1-en-4,6-diyn-3-yl acetate, 8-hydroxynonacosa-1-en-4,6-diyn-3-yl acetate, (3S,8R)-heptadeca-1-en-4,6-diyne-3,8-diyl diacetate, (3S,8S)-heptadeca-1-en-4,6-diyne-3,8-diyl diacetate, (3R,8R)-heptadeca-1-en-4,6-diyne-3,8-diyl diacetate, (3R,8S)-heptadeca-1-en-4,6-diyne-3,8-diyl diacetate, (3S,8R)-3-methoxyheptadeca-1-en-4,6-diyn-8-ol, (3S,8S)-3-methoxyheptadeca-1-en-4,6-diyn-8-ol, (3R,8R)-3-methoxyheptadeca-1-en-4,6-diyn-8-ol, (3R,8S)-3-methoxyheptadeca-1-en-4,6-diyn-8-ol, 8-((methylsulfonyl)oxy)heptadeca-1-en-4,6-diyn-3-yl acetate, 8-(tosyloxy)heptadeca-1-en-4,6-diyn-3-yl acetate, 8-acetamidoheptadeca-1-en-4,6-diyn-3-yl acetate, 8-(methylsulfonamido)heptadeca-1-en-4,6-diyn-3-yl acetate, 8-phenoxyheptadeca-1-en-4,6-diyn-3-yl acetate, 8-(acetylthio)heptadeca-1-en-4,6-diyn-3-yl acetate, 8-bromoheptadeca-1-en-4,6-diyn-3-yl acetate, (3S,8R)-8-(4-chlorobenzamido)heptadeca-1-en-4,6-diyn-3-yl acetate, (3S,8R)-8-(3,4-dichlorobenzamido)heptadeca-1-en-4,6-diyn-3-yl acetate, (3S,8R)-8-(4-methylbenz amido)heptadeca-1-en-4,6-diyn-3-yl acetate, (3S,8R)-8-(4-methoxybenzamido)heptadeca-1-en-4,6-diyn-3-yl acetate, (3S,8R)-8-hydroxyundeca-1-en-4,6-diyn-3-yl acetate, (3S,8R)-8-hydroxytricosa-1-en-4,6-diyn-3-yl acetate, (3S,8R)-8-aminoheptadeca-1-en-4,6-diyn-3-yl acetate, (3S,8R)-8-(methylsulfonamido)heptadeca-1-en-4,6-diyn-3-yl acetate, (3S,8R)-8-benzamidoheptadeca-1-en-4,6-diyn-3-yl acetate, (3S,8R)-8-(butylamino)heptadeca-1-en-4,6-diyn-3-yl acetate, (3S,8R)-8-((methylsulfonyl)oxy)heptadeca-1-en-4,6-diyn-3-yl acetate, (3S,8R)-heptadeca-1-en-4,6-diyne-3,8-diol, (3S,8S)-heptadeca-1-en-4,6-diyne-3,8-diol, (3R,8R)-heptadeca-1-en-4,6-diyne-3,8-diol, (3R,8S)-heptadeca-1-en-4,6-diyne-3,8-diol, (3S,8R)-8-aminoheptadeca-1-en-4,6-diyn-3-ol, (3S,8R)-8-(butylamino)heptadeca-1-en-4,6-diyn-3-ol, N-((3S,8R)-3-hydroxyheptadeca-1-en-4,6-diyn-8-yl)benzamide, ethyl((3S,8R)-3-hydroxyheptadeca-1-en-4,6-diyn-8-yl)carbamate, (3S,8R)-8-methoxyheptadeca-1-en-4,6-diyn-3-ol, N-((3S,8R)-3-hydroxyheptadeca-1-en-4,6-diyn-8-yl)acetamide, or (3S,8R)-3-hydroxyheptadeca-1-en-4,6-diyn-8-yl acetate. A list showing the chemical structure of these exemplary compounds is provided in FIG. 1.

The compounds of the invention are useful for the treatment or prevention of protozoal infections in a patient (e.g., a human) such as, e.g., leishmaniasis and malaria. As described herein, the compounds of the invention exert potent antiprotozoal activity against Leishmania, the causative agent of leishmaniasis. Similarly, the compounds of the invention are effective inhibitors of Plasmodium, the causative agent of malaria. As described by example herein, the compounds of the invention exhibit inhibitory growth effects on protozoa-infected cell cultures without substantial bystander cytotoxicity.

The compounds of the invention are also useful for the treatment or prevention of microbial infections in a patient (e.g., a human) such as, e.g., a bacterial or yeast infection. As described herein, the compounds of the invention display antimicrobial activity against a panel of bacteria and yeast that included S. aureus, E. faecalis, S. pyogenes, C. glabrata, B. subtilis, P. aeruginosa, B. anthracis, and C. albicans, each the causative agent of human or animal disease.

The compounds of the invention are further useful for the treatment or prevention of cancer in a patient (e.g., a human) such as, e.g., squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, breast cancer, colon cancer, colorectal cancer, endometrial carcinoma, salivary gland carcinoma, kidney cancer, renal cancer, prostate cancer, vulval cancer, thyroid cancer, hepatic carcinoma, and head and neck cancer. As described herein, the compounds of the invention display broad anti-cancer activity by inhibiting the in vitro growth of various human tumor cell lines.

### B. Methods of Treatment

The compounds of the invention can be used to treat a patient (e.g., a human) that suffers from or is at risk of suffering from a disease, disorder, condition or symptom caused by a protozoal or microbial infection or a cancer. The compounds of the invention can be used alone or in combination with other agents and compounds in methods of treating or preventing such infections or cancer. Each such treatment described above includes the step of administering to a patient in need thereof a therapeutically effective amount of the compound of the invention described herein to delay, reduce or prevent such disease, disorder, condition, or symptom.

Besides being useful for human treatment, the compounds and formulations of the present invention are also useful for the treatment or prevention of protozoal and microbial infections and cancer in animals, e.g., the veterinary treatment of domesticated animal, companion animals (e.g., dogs and cats), exotic animals, farm animals (e.g., ungulates, including horses, cows, sheep, goats, and pigs), and animals used in scientific research (e.g., rodents).

### C. Compound Administration and Formulation

Basic addition salts can be prepared during the final isolation and purification of the compounds by reaction of a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid. The novel compounds described herein can be prepared in a form of pharmaceutically acceptable salts that will be prepared from nontoxic inorganic or organic bases including but not limited to aluminum, ammonium, calcium, copper, ferric, ferrous, lithium, magnesium, manganic salts, manganous, potassium, sodium, zinc, and the like. Salts derived from pharmaceutically acceptable organic non-toxic bases include salts of primary, secondary, and tertiary amines, substituted amines including naturally-occurring substituted amines, cyclic amines, and basic ion exchange resins, such as arginine, betaine, caffeine, choline, ethylamine, 2-diethylaminoethano, 1,2-dimethylaminoethanol, ethanolamine, ethylenediamine, N-ethyl-morpholine, N-ethylpiperidine, glucamine, glucosamine, histidine, hydroxylamine, isopropylamine, lysine, methylglucamine, morpholine, piperazine, piperidine, polyamine resins, procaine, purines, theobromine, triethylamine, trimethylamine, trishydroxylmethyl amino methane, tripropyl amine, and tromethamine.

If the compounds of the invention are basic, salts could be prepared in a form of pharmaceutically acceptable salts that will be prepared from nontoxic inorganic or organic acids including but not limited to hydrochloric, hydrobromic, phosphoric, sulfuric, tartaric, citric, acetic, fumaric, alkylsulphonic, naphthalenesulphonic, para-toluenesulphonic, camphoric acids, benzenesulfonic, benzoic, camphorsulfonic, citric, ethanesulfonic, gluconic, glutamic, isethonic, lactic, maleic, malic, mandelic, methanesulfonic, mucic, nitric, pamoic, pantothenic, phosphoric, and succinic.

While it may be possible for the compounds of the invention to be administered as the raw chemical, it is also possible to present them as a pharmaceutical formulation. Accordingly, the present invention provides a pharmaceutical formulation comprising a compound or a pharmaceutically acceptable salt, ester, prodrug or solvate thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions of the present invention may be manufactured in a manner that is itself known, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing into association a compound of the present invention or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds of the invention may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds of the invention may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

For buccal or sublingual administration, the compounds of the invention may take the form of tablets, lozenges, pastilles, or gels formulated in conventional manner. Such compositions may comprise the active ingredient in a flavored basis such as sucrose and acacia or tragacanth.

The compounds of the invention may also be formulated in rectal compositions such as suppositories or retention enemas, e.g., containing conventional suppository bases such as cocoa butter, polyethylene glycol, or other glycerides.

Compounds of the invention may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include solid, liquid or semi-liquid preparations suitable for penetration through the skin to the site of inflammation or infection such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation. It may however comprise as much as 10% w/w but preferably will comprise less than 5% w/w, more preferably from 0.1% to 1% w/w of the formulation.

Via the topical route, the pharmaceutical composition according to the invention may be in the form of liquid or semi liquid such as ointments, or in the form of solid such as powders. It may also be in the form of suspensions such as polymeric microspheres, or polymer patches and hydrogels allowing a controlled release. This topical composition may be in anhydrous form, in aqueous form or in the form of an emulsion. The compounds are used topically at a concentration generally of between 0.001% and 10% by weight and preferably between 0.01% and 1% by weight, relative to the total weight of the composition.

For administration by inhalation, the compounds according to the invention are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavoring agents.

The compounds of the invention may be administered orally or via injection at a dose of from 0.1 to 500 mg/kg per day. The dose range for adult humans is generally from 5 mg to 2 g/day. Tablets or other forms of presentation provided in discrete units may conveniently contain an amount of compound of the invention which is effective at such dosage or as a multiple of the same, for instance, units containing 5 mg to 500 mg, usually around 10 mg to 200 mg.

Compounds according to the invention can be administered at a daily dose of about 0.001 mg/kg to 100 mg/kg of body weight, in 1 to 3 dosage intakes. Further, compounds can be used systemically, at a concentration generally of between 0.001% and 10% by weight and preferably between 0.01% and 1% by weight, relative to the weight of the composition.

The amount of active ingredient that may be combined with the carrier materials to produce a single dosage form will vary depending upon the host treated and the particular mode of administration.

The compounds of the invention can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds of the invention described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit experienced by a patient may be increased by administering one of the compounds described herein with another therapeutic agent (which also includes a therapeutic regimen) that also has therapeutic benefit. By way of example only, in a treatment for pain involving administration of one of the compounds described herein, increased therapeutic benefit may result by also providing the patient with another therapeutic agent for pain. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of the compounds of the invention together with inert or active compounds, or other drugs including wetting agents, flavor enhancers, preserving agents, stabilizers, humidity regulators, pH regulators, osmotic pressure modifiers, emulsifiers, UV-A and UV-B screening agents, antioxidants, depigmenting agents such as hydroquinone or kojic acid, emollients, moisturizers, for instance glycerol, PEG 400, or urea, antiseborrhoeic or antiacne agents, such as S-carboxymethylcysteine, S-benzylcysteamine, salts thereof or derivatives thereof, or benzoyl peroxide, antibiotics, for instance erythromycin and tetracyclines, chemotherapeutic agent, for example, paclitaxel, antifungal agents such as ketoconazole, agents for promoting regrowth of the hair, for example, minoxidil (2,4-diamino-6-piperidinopyrimidine 3-oxide), non-steroidal anti-inflammatory agents, carotenoids, and especially p-carotene, antipsoriatic agents such as anthralin and its derivatives, eicosa-5,8,11,14-tetraynoic acid and eicosa-5,8,11-triynoic acid, and esters and amides thereof, retinoids, e.g., RAR or RXR receptor ligands, which may be natural or synthetic, corticosteroids or oestrogens, alpha-hydroxy acids and a-keto acids or derivatives thereof, such as lactic acid, malic acid, citric acid, and also the salts, amides or esters thereof, or p-hydroxy acids or derivatives thereof, such as salicylic acid and the salts, amides or esters thereof, ion-channel blockers such as potassium-channel blockers, or alternatively, more particularly for the pharmaceutical compositions, in combination with medicaments known to interfere with the immune system, anticonvulsant agents include, and are not limited to, topiramate, analogs of topiramate, carbamazepine, valproic acid, lamotrigine, gabapentin, phenyloin and the like and mixtures or pharmaceutically acceptable salts thereof. A person skilled in the art will take care to select the other compound(s) to be added to these compositions such that the advantageous properties intrinsically associated with the compounds of the invention are not, or are not substantially, adversely affected by the envisaged addition.

In any case, the multiple therapeutic agents (at least one of which is a compound of the present invention) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, methods for treating diseases, disorders, conditions, or symptoms in a patient (e.g., a human or animal) in need of such treatment are presented herein, the methods comprising the step of administering to the patient an amount of a compound of the invention effective to reduce or prevent the disease, disorder, condition, or symptom, in combination with at least one additional agent for the treatment of said disorder that is known in the art.

### D. Therapeutic Kits

As a matter of convenience, the compounds of the present invention can be provided in a kit, i.e., a packaged combination of reagents in predetermined amounts with instructions for treating a patient (e.g., a human). Additives such as stabilizers, buffers and the like may be included. The relative amounts of the various reagents may be varied widely to provide for concentrations in solution of the reagents which substantially optimize the clinical efficacy of the compounds.

### E. Examples

It is understood that the foregoing examples are merely illustrative of the present invention. Certain modifications of the articles and/or methods employed may be made and still achieve the objectives of the invention. Such modifications are contemplated as within the scope of the claimed invention.

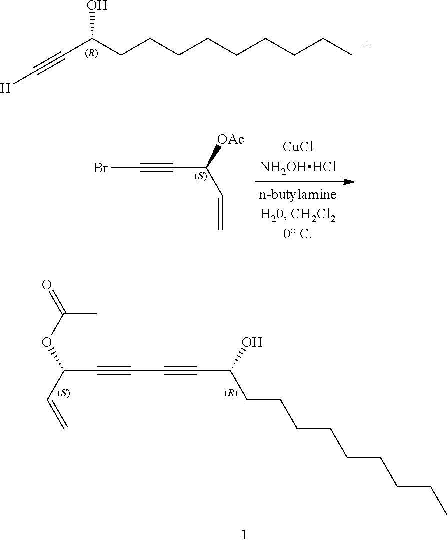

### Example 1

### Synthesis of (3S,8R)-8-hydroxyheptadeca-1-en-4,6-diyn-3-yl acetate (compound 1)

Copper (I) chloride (0.0070 g, 0.07 mmol) was added to a stirred 30% solution (1.65 mL) of n-butylamine in distilled water at 0° C. which resulted in a deep blue solution. A few crystals of NH₂OH.HCl were added until solution was colorless. A solution of (R)-dodec-1-yn-3-ol (0.1051 g, 0.58 mmol) in CH₂Cl₂ (0.86 mL) was added under argon atmosphere which resulted in yellow reaction mixture. After 10 minutes, a solution of (S)-5-bromopent-1-en-4-yn-3-yl acetate (0.2350 g, 1.16 mmol) in CH₂Cl₂ (1.7 mL) was added dropwise over 3 minutes. A few crystals of NH₂OH.HCl were added as necessary whenever solution turned blue or green. After 20 minutes, reaction was quenched with water, extracted 3 times with CH₂Cl₂, dried over Na₂SO₄, and concentrated under reduced pressure. Immediate purification by flash chromatography on silica gel (hexanes—15% EtOAc/hexanes) afforded compound 1 (102.9 mg, 58.6%) as a yellow oil.

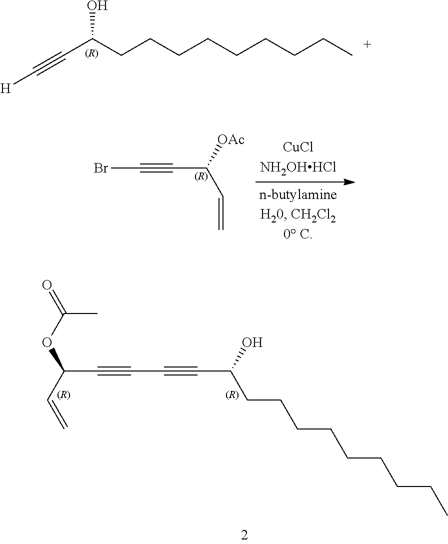

### Example 2

### Synthesis of (3R,8R)-8-hydroxyheptadeca-1-en-4,6-diyn-3-yl acetate (compound 2)

Copper (I) chloride (0.0081 g, 0.08 mmol) was added to a stirred 30% solution (1.65 mL) of n-butylamine in distilled water at 0° C. which resulted in a deep blue solution. A few crystals of NH₂OH.HCl were added until solution was colorless. A solution of (R)-dodec-1-yn-3-ol (0.0976 g, 0.54 mmol) in CH₂Cl₂ (0.81 mL) was added under argon atmosphere which resulted in yellow reaction mixture. After 10 minutes, a solution of (R)-5-bromopent-1-en-4-yn-3-yl acetate (0.2297 g, 1.13 mmol) in CH₂Cl₂ (1.7 mL) was added dropwise over 4 minutes. A few crystals of NH₂OH.HCl were added as necessary whenever solution turned blue or green. After 20 minutes, reaction was quenched with water, extracted 3 times with CH₂Cl₂, dried over Na₂SO₄, and concentrated under reduced pressure. Immediate purification by flash chromatography on silica gel (hexanes—15% EtOAc/hexanes) afforded compound 2 (87.8 mg, 53.9%) as a yellow oil.

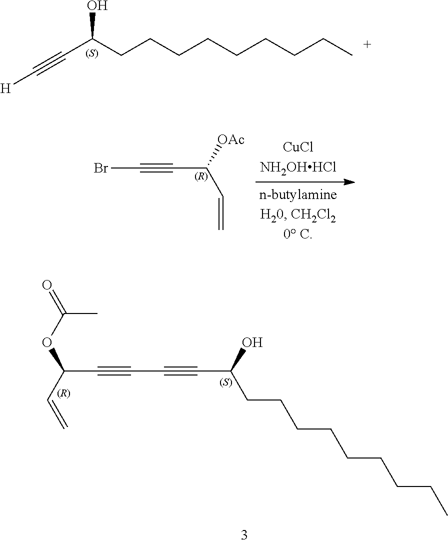

### Example 3

### Synthesis of (3R,85)-8-hydroxyheptadeca-1-en-4,6-diyn-3-yl acetate (compound 3)

Copper (I) chloride (0.0071 g, 0.07 mmol) was added to a stirred 30% solution (1.65 mL) of n-butylamine in distilled water at 0° C. which resulted in a deep blue solution. A few crystals of NH₂OH.HCl were added until solution was colorless. A solution of (S)-dodec-1-yn-3-ol (0.1067 g, 0.59 mmol) in CH₂Cl₂ (0.89 mL) was added under argon atmosphere which resulted in yellow reaction mixture. After 10 minutes, a solution of (R)-5-bromopent-1-en-4-yn-3-yl acetate (0.1466 g, 0.72 mmol) in CH₂Cl₂ (1.1 mL) was added dropwise over 5 minutes. A few crystals of NH₂OH.HCl were added as necessary whenever solution turned blue or green. After 20 minutes, reaction was quenched with water, extracted 3 times with CH₂Cl₂, dried over Na₂SO₄, and concentrated under reduced pressure. Immediate purification by flash chromatography on silica gel (hexanes—15% EtOAc/hexanes) afforded compound 3 (127.5 mg, 71.5%) as a yellow oil.

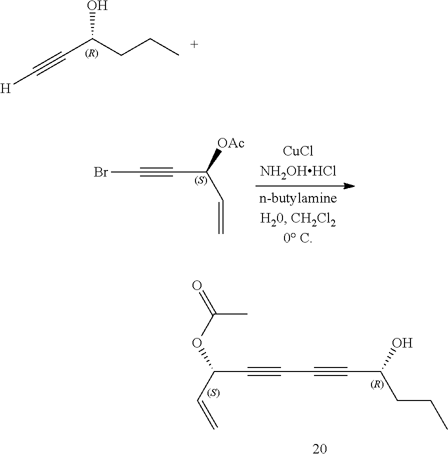

### Example 4

### Synthesis of (3S,8R)-8-hydroxyundeca-1-en-4,6-diyn-3-yl acetate (compound 20)

Copper (I) chloride (0.01 g, 0.10 mmol) was added to a stirred 30% solution (3.1 mL) of n-butylamine in distilled water at 0° C. which resulted in a deep blue solution. A few crystals of NH₂OH.HCl were added until solution was colorless. A solution of (R)-hex-1-yn-3-ol (0.100 g, 1.02 mmol) in CH₂Cl₂ (1.5 mL) was added under argon atmosphere which resulted in yellow reaction mixture. After 10 minutes, a solution of (S)-5-bromopent-1-en-4-yn-3-yl acetate (0.25 g, 1.2 mmol) in CH₂Cl₂ (1.7 mL) was added dropwise over 5 minutes. A few crystals of NH₂OH.HCl were added as necessary whenever solution turned blue or green. After 1.5 hours, reaction was quenched with water, extracted 3 times with CH₂Cl₂, dried over Na₂SO₄, and concentrated under reduced pressure. Immediate purification by flash chromatography on silica gel (hexanes—15% EtOAc/hexanes) afforded compound 20 as a yellow oil.

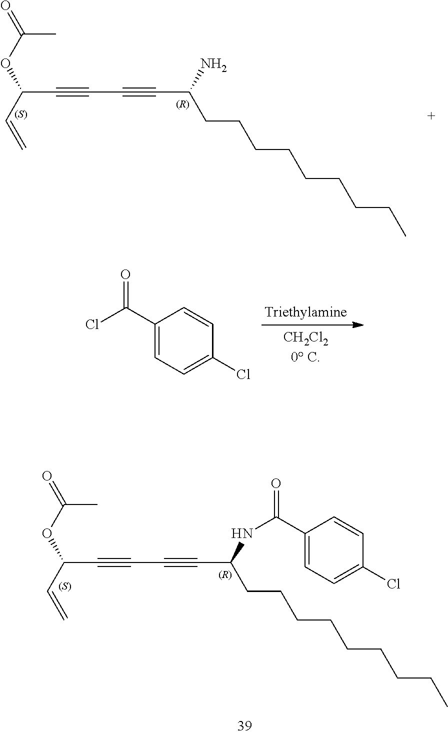

### Example 5

### Synthesis of (3S,8R)-8-(4-chlorobenzamido)heptadeca-1-en-4,6-diyn-3-yl acetate (compound 39)

To a solution of compound 45 (0.056 g, 0.19 mmol) in CH₂Cl₂ (1.8 mL) at 0° C. under Ar atmosphere was added triethylamine (0.04 mL, 0.29 mmol) followed by a solution of 4-chlorobenzoyl chloride (0.05 g, 0.29 mmol) in CH₂Cl₂ (1 mL). Reaction mixture was stirred at 0° C. for 3 hours and 15 minutes. Reaction was then quenched with water, extracted three times with CH₂Cl₂, washed with saturated NaHCO₃, dried over Na₂SO₄, and concentrated under reduced pressure. Purification by flash chromatography on silica gel (hexanes—15% EtOAc/hexanes) afforded compound 39 (0.028 g, 34.4%) as a yellow oil.

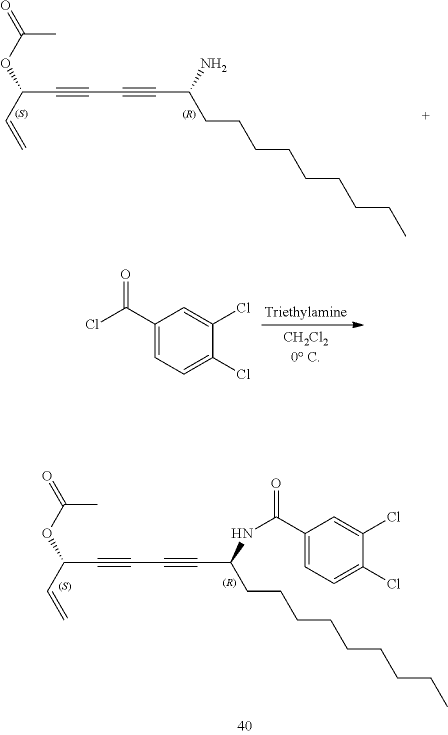

### Example 6

### Synthesis of (3S,8R)-8-(3,4-dichlorobenzamido)heptadeca-1-en-4,6-diyn-3-yl acetate (compound 40)

To a solution of compound 45 (0.060 g, 0.20 mmol) in CH₂Cl₂ (2.0 mL) at 0° C. under Ar atmosphere was added triethylamine (0.03 mL, 0.24 mmol) followed by a solution of 3,4-dichlorobenzoyl chloride (0.051 g, 0.25 mmol) in CH₂Cl₂ (1 mL). Reaction mixture was stirred at 0° C. for 1.5 hours. Reaction was then quenched with water, extracted three times with CH₂Cl₂, washed with saturated NaHCO₃, dried over Na₂SO₄, and concentrated under reduced pressure. Purification by flash chromatography on silica gel (hexanes—15% EtOAc/hexanes) afforded compound 40 (0.039 g, 41.1%) as a yellow oil.

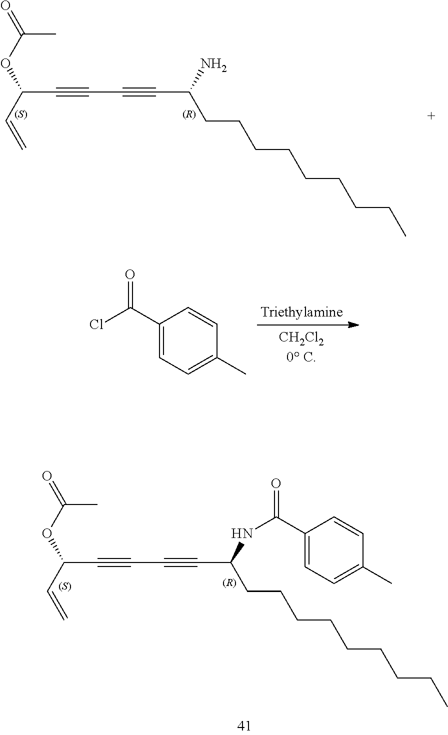

### Example 7

### Synthesis of (3S,8R)-8-(4-methylbenzamido)heptadeca-1-en-4,6-diyn-3-yl acetate (compound 41)

To a solution of compound 45 (0.032 g, 0.10 mmol) in CH₂Cl₂ (1.0 mL) at 0° C. under Ar atmosphere was added triethylamine (0.06 mL, 0.43 mmol) followed by a solution of p-toluoyl chloride (0.02 mL, 0.15 mmol) in CH₂Cl₂ (1.0 mL). Reaction mixture was stirred at 0° C. for 45 minutes. Reaction was then quenched with water, extracted three times with CH₂Cl₂, dried over Na₂SO₄, and concentrated under reduced pressure. Purification by flash chromatography on silica gel (hexanes—15% EtOAc/hexanes) afforded compound 41 but it was contaminated with p-toluic acid per GC/MS analysis. Diluted product with ether, washed four times with saturated NaHCO₃, washed once with brine, dried over Na₂SO₄, and concentrated under reduced pressure which afforded purified compound 41 (0.030 g, 67.7%, yellow oil).

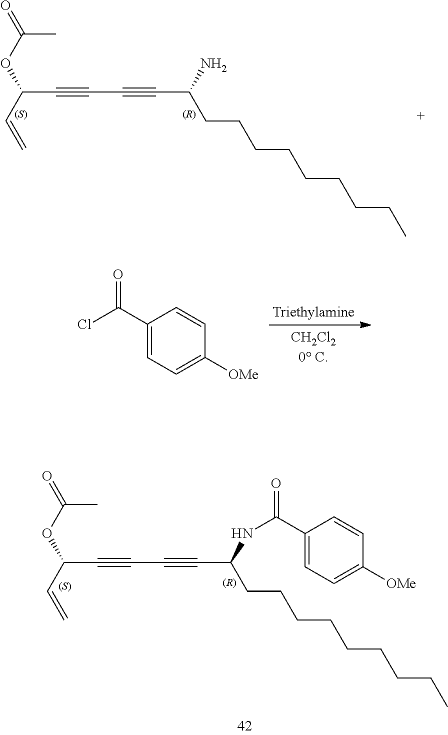

### Example 8

### Synthesis of (3S,8R)-8-(4-methoxybenzamido)heptadeca-1-en-4,6-diyn-3-yl acetate (compound 42)

To a solution of compound 45 (0.045 g, 0.15 mmol) in CH₂Cl₂ (2.0 mL) at 0° C. under Ar atmosphere was added triethylamine (0.06 mL, 0.43 mmol) followed by a solution of 4-methoxybenzoyl chloride (0.043 g, 0.25 mmol) in CH₂Cl₂ (1 mL). Reaction mixture was stirred at 0° C. for 1 hour and 50 minutes. Reaction was then quenched with water, extracted three times with CH₂Cl₂, washed with saturated NaHCO₃, dried over Na₂SO₄, and concentrated under reduced pressure. Purification by flash chromatography on silica gel (hexanes—15% EtOAc/hexanes) afforded compound 42 (0.014 g, 21.8%) as a yellow oil.

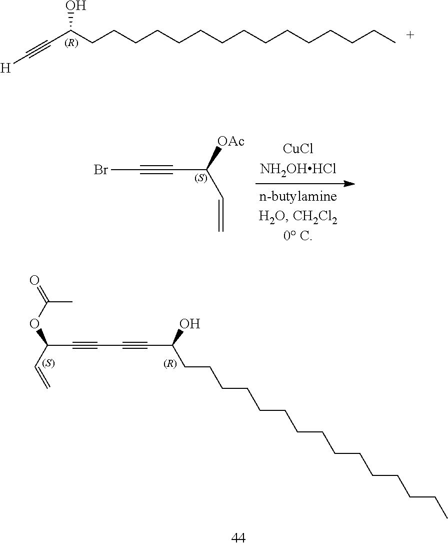

### Example 9

### Synthesis of (3S,8R)-8-hydroxytricosa-1-en-4,6-diyn-3-yl acetate (compound 44)

Copper (I) chloride (0.0037 g, 0.04 mmol) was added to a stirred 30% solution (0.90 mL) of n-butylamine in distilled water at 0° C. which resulted in a deep blue solution. A few crystals of NH₂OH.HCl were added until solution was colorless. A solution of (R)-octadec-1-yn-3-ol (0.0740 g, 0.28 mmol) in CH₂Cl₂ (0.45 mL) was added under argon atmosphere which resulted in yellow reaction mixture. After 10 minutes, a solution of (S)-5-bromopent-1-en-4-yn-3-yl acetate (0.0685 g, 0.34 mmol) in CH₂Cl₂ (0.54 mL) was added dropwise over 5 minutes. A few crystals of NH₂OH.HCl were added as necessary whenever solution turned blue or green. After 25 minutes, reaction was quenched with water, extracted 3 times with CH₂Cl₂, dried over Na₂SO₄, and concentrated under reduced pressure. Immediate purification by flash chromatography on silica gel (hexanes—15% EtOAc/hexanes) afforded compound 44 (58.2 mg, 53.9%) as a light orange solid.

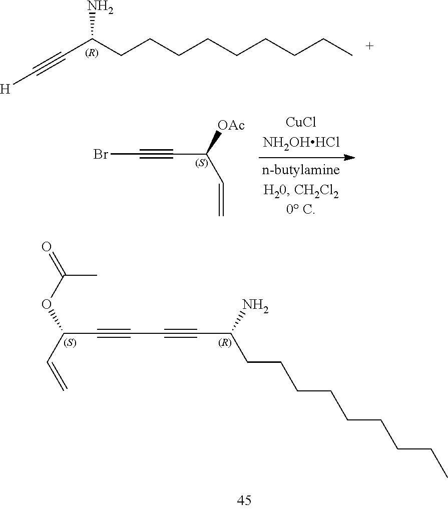

### Example 10

### Synthesis of (3S,8R)-8-aminoheptadeca-1-en-4,6-diyn-3-yl acetate (compound 45)

Copper (I) chloride (0.0242 g, 0.24 mmol) was added to a stirred 30% solution (5.0 mL) of n-butylamine in distilled water at 0° C. which resulted in a deep blue solution. A few crystals of NH₂OH.HCl were added until solution was colorless. A solution of (R)-dodec-1-yn-3-amine (0.3053 g, 1.68 mmol) in CH₂Cl₂ (2.5 mL) was added under argon atmosphere which resulted in yellow reaction mixture. After 10 minutes, a solution of (S)-5-bromopent-1-en-4-yn-3-yl acetate (0.4080 g, 2.01 mmol) in CH₂Cl₂ (3.0 mL) was added dropwise over 10 minutes. A few crystals of NH₂OH.HCl were added as necessary whenever solution turned blue or green. After 45 minutes, reaction was quenched with water, extracted 3 times with CH₂Cl₂, dried over Na₂SO₄, and concentrated under reduced pressure. Immediate purification by flash chromatography on silica gel (hexanes—60% EtOAc/hexanes) afforded compound 45 (0.1415 g, 27.7%) as an orange-brown oil.

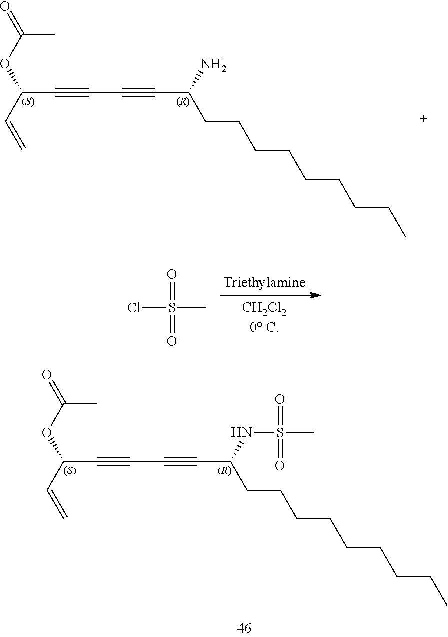

### Example 11

### Synthesis of (3S,8R)-8-(methylsulfonamido)heptadeca-1-en-4,6-diyn-3-yl acetate (compound 46)

To a solution of compound 45 (0.0416 g, 0.14 mmol) in CH₂Cl₂ (0.94 mL) at 0° C. under Ar atmosphere was added triethylamine (0.040 mL, 0.29 mmol) followed by methanesulfonyl chloride (0.012 mL, 0.16 mmol). After 1 hour and 10 minutes, reaction was quenched with water, extracted three times with CH₂Cl₂, dried over Na₂SO₄, and concentrated under reduced pressure. Purification by flash chromatography on silica gel (hexanes—25% EtOAc/hexanes) afforded compound 46 (29.2 mg, 55.8%) as a yellow oil.

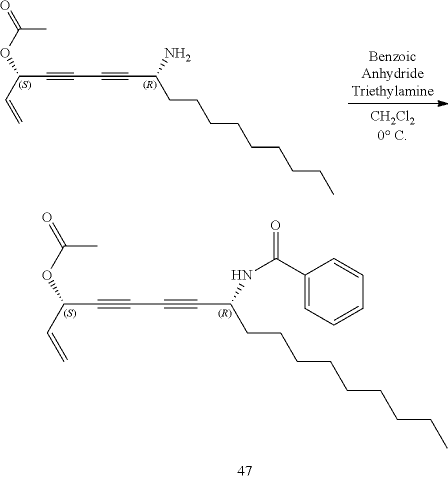

### Example 12

### Synthesis of (3S,8R)-8-benzamidoheptadeca-1-en-4,6-diyn-3-yl acetate (compound 47)

To a solution of compound 45 (0.0455 g, 0.15 mmol) in CH₂Cl₂ (1.6 mL) at 0° C. under Ar was added benzoic anhydride (0.0467 g, 0.21 mmol) followed by triethylamine (0.030 mL, 0.22 mmol). Reaction mixture was stirred for 15 minutes at 0° C. and then allowed to warm to ambient temperature. After 1 hour and 15 minutes, reaction mixture was quenched with water, extracted three times with CH₂Cl₂, dried over Na₂SO₄, and concentrated under reduced pressure. Purification by flash chromatography on silica gel (hexanes—15% EtOAc/hexanes) afforded compound 47 (31.3 mg, 51.2%) as a beige oil.

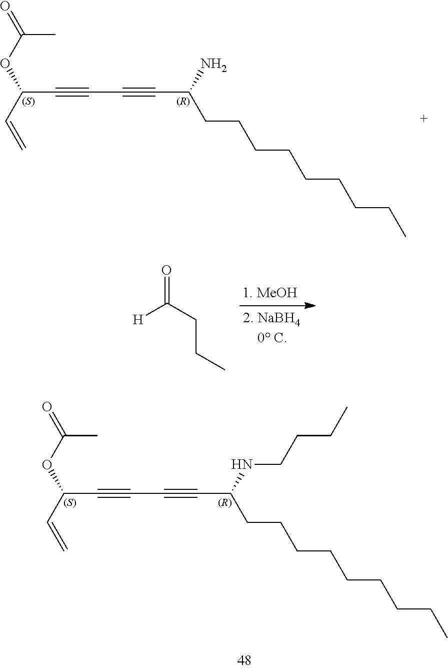

### Example 13

### Synthesis of (3S,8R)-8-(butylamino)heptadeca-1-en-4,6-diyn-3-yl acetate (compound 48)

To a solution of compound 45 (0.0493 g, 0.16 mmol) in methanol (0.81 mL) under argon atmosphere was added butyraldehyde (0.021 mL, 0.23 mmol). Reaction mixture was stirred at ambient temperature for 15 minutes and then chilled to 0° C. at which time NaBH4 was added (0.0050 g, 0.13 mmol). Reaction mixture was stirred for 1.5 hours and then diluted with EtOAc, washed with saturated NaHCO₃, dried over Na₂SO₄, and concentrated under reduced pressure. Purification by flash chromatography on silica gel (hexanes—10% EtOAc/hexanes) afforded compound 48 (20.6 mg, 35.3%) as a yellow oil.

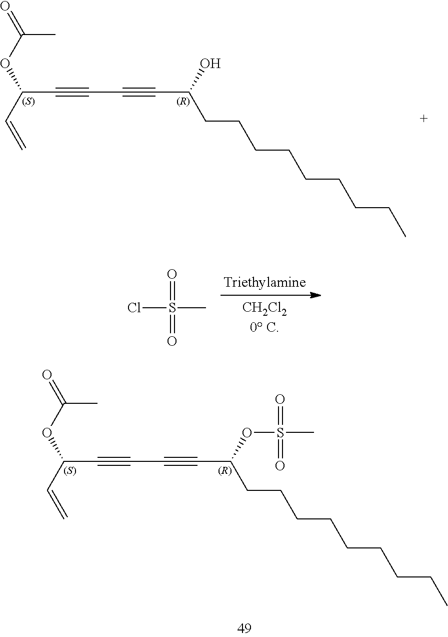

### Example 14

### Synthesis of (3S,8R)-8-((methylsulfonyl)oxy)heptadeca-1-en-4,6-diyn-3-yl acetate (compound 49)

To a solution of compound 1 (0.0273 g, 0.09 mmol) in CH₂Cl₂ (0.30 mL) at 0° C. under Ar atmosphere was added triethylamine (0.020 mL, 0.14 mmol) followed by methanesulfonyl chloride (0.010 mL, 0.13 mmol). After 1 hour, reaction was quenched with water, extracted three times with CH₂Cl₂, dried over Na₂SO₄, and concentrated under reduced pressure. Purification by flash chromatography on silica gel afforded compound 49 (21.4 mg, 62.4%) as a beige oil.

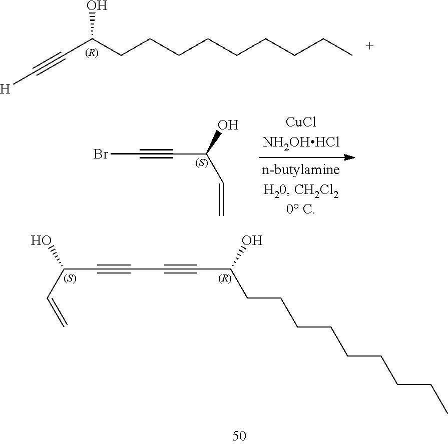

### Example 15

### Synthesis of (3S,8R)-heptadeca-1-en-4,6-diyne-3,8-diol (compound 50)

Copper (I) chloride (0.0112 g, 0.11 mmol) was added to a stirred 30% solution (1.6 mL) of n-butylamine in distilled water at 0° C. which resulted in a deep blue solution. A few crystals of NH₂OH.HCl were added until solution was colorless. A solution of (R)-dodec-1-yn-3-ol (0.1050 g, 0.58 mmol) in CH₂Cl₂ (0.86 mL) was added under argon atmosphere which resulted in yellow reaction mixture. After 10 minutes, a solution of (S)-5-bromopent-1-en-4-yn-3-ol (0.1548 g, 0.96 mmol) in CH₂Cl₂ (1.4 mL) was added dropwise over 5 minutes. A few crystals of NH₂OH.HCl were added as necessary whenever solution turned blue or green. After 55 minutes, reaction was quenched with water, extracted 3 times with CH₂Cl₂, dried over Na₂SO₄, and concentrated under reduced pressure. Immediate purification by flash chromatography on silica gel (15% EtOAc/hexanes) afforded compound 50 (45.9 mg, 30.4%) as a reddish-brown oil.

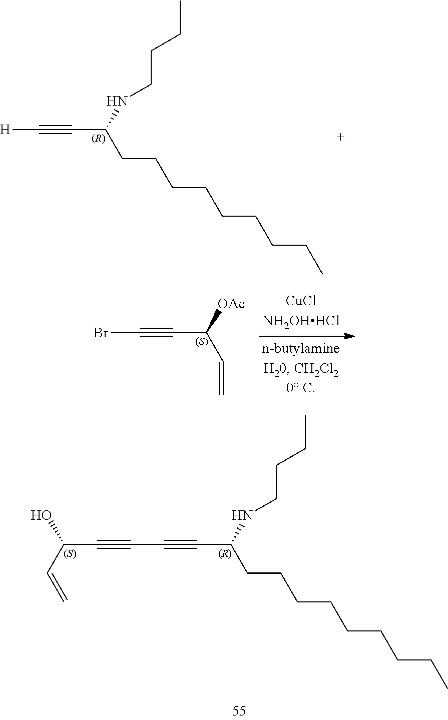

### Example 16

### Synthesis of (3S,8R)-8-(butylamino)heptadeca-1-en-4,6-diyn-3-ol (compound 55)

Copper (I) chloride (0.006 g, 0.06 mmol) was added to a stirred 30% solution (1.1 mL) of n-butylamine in distilled water at 0° C. which resulted in a deep blue solution. A few crystals of NH₂OH.HCl were added until solution was colorless. A solution of (R)—N-butyldodec-1-yn-3-amine (0.087 g, 0.37 mmol) in CH₂Cl₂ (0.55 mL) was added under argon atmosphere which resulted in yellow reaction mixture. After 10 minutes, a solution of (S)-5-bromopent-1-en-4-yn-3-ol (0.118 g, 0.73 mmol) in CH₂Cl₂ (1.1 mL) was added dropwise over 5 minutes. A few crystals of NH₂OH.HCl were added as necessary whenever solution turned blue or green. After 45 minutes, reaction was quenched with water, extracted 3 times with CH₂Cl₂, dried over Na₂SO₄, and concentrated under reduced pressure. Immediate purification by flash chromatography on silica gel (hexanes—15% EtOAc/hexanes) afforded compound 55 (0.063 g, 53.9%, tan solid).

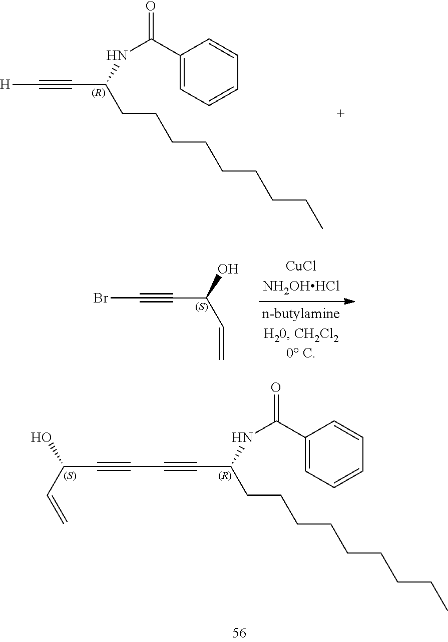

### Example 17

### Synthesis of N-((3S,8R)-3-hydroxyheptadeca-1-en-4,6-diyn-8-yl)benzamide (compound 56)

Copper (I) chloride (0.004 g, 0.038 mmol) was added to a stirred 30% solution (0.9 mL) of n-butylamine in distilled water at 0° C. which resulted in a deep blue solution. A few crystals of NH₂OH.HCl were added until solution was colorless. A solution of (R)—N-(dodec-1-yn-3-yl)benzamide (0.086 g, 0.30 mmol) in CH₂Cl₂ (0.5 mL) was added under argon atmosphere which resulted in yellow reaction mixture. After 10 minutes, a solution of (S)-5-bromopent-1-en-4-yn-3-ol (0.081 g, 0.50 mmol) in CH₂Cl₂ (0.7 mL) was added dropwise over 5 minutes. A few crystals of NH₂OH.HCl were added as necessary whenever solution turned blue or green. After 45 minutes, analysis of the reaction mixture by GC/MS showed that there was still some (R)—N-(dodec-1-yn-3-yl)benzamide present. At 1 hour and 15 minutes, added additional (S)-5-bromopent-1-en-4-yn-3-ol (0.036 g, 0.22 mmol) in CH₂Cl₂ (0.5 mL). Reaction was stirred an additional 15 minutes and then quenched with water, extracted 3 times with CH₂Cl₂, dried over Na₂SO₄, and concentrated under reduced pressure. Immediate purification by flash chromatography on silica gel (Hexanes—25% EtOAc/hexanes) afforded compound 56 (0.105 g, 95.3%) as a beige oil.

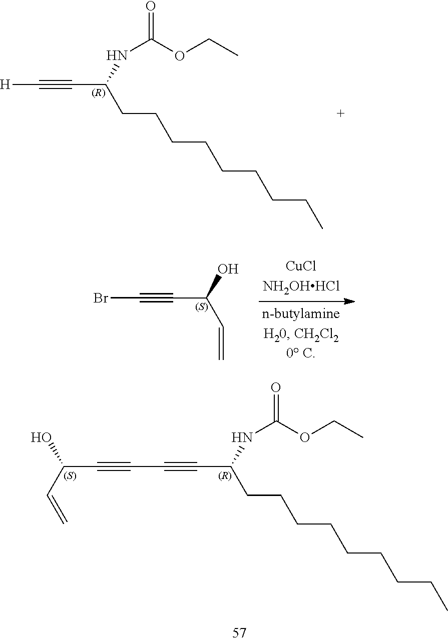

### Example 18

### Synthesis of ethyl((3S,8R)-3-hydroxyheptadeca-1-en-4,6-diyn-8-yl)carbamate (compound 57)

Copper (I) chloride (0.007 g, 0.07 mmol) was added to a stirred 30% solution (1.0 mL) of n-butylamine in distilled water at 0° C. which resulted in a deep blue solution. A few crystals of NH₂OH.HCl were added until solution was colorless. A solution of (R)-ethyl dodec-1-yn-3-ylcarbamate (0.084 g, 0.33 mmol) in CH₂Cl₂ (0.5 mL) was added under argon atmosphere which resulted in yellow reaction mixture. After 10 minutes, a solution of (S)-5-bromopent-1-en-4-yn-3-ol (0.067 g, 0.42 mmol) in CH₂Cl₂ (0.6 mL) was added dropwise over 5 minutes. A few crystals of NH₂OH.HCl were added as necessary whenever solution turned blue or green. After 16 minutes, analysis of the reaction mixture by GC/MS showed that there was still some (R)-ethyl dodec-1-yn-3-ylcarbamate present. At 50 minutes, added additional (S)-5-bromopent-1-en-4-yn-3-ol (0.043 g, 0.27 mmol) in CH₂Cl₂ (0.5 mL). Reaction was stirred an additional 1 hour and then quenched with water, extracted 3 times with CH₂Cl₂, dried over Na₂SO₄, and concentrated under reduced pressure. Immediate purification by flash chromatography on silica gel (hexanes—10% EtOAc/hexanes) afforded compound 57 (0.065 g, 58.4%, brown-orange solid).

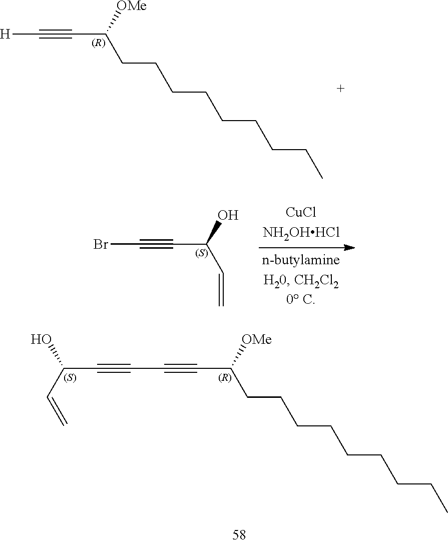

### Example 19

### Synthesis of (3S,8R)-8-methoxyheptadeca-1-en-4,6-diyn-3-ol (compound 58)

Copper (I) chloride (0.006 g, 0.06 mmol) was added to a stirred 30% solution (1.5 mL) of n-butylamine in distilled water at 0° C. which resulted in a deep blue solution. A few crystals of NH₂OH.HCl were added until solution was colorless. A solution of (R)-3-methoxydodec-1-yne (0.102 g, 0.52 mmol) in CH₂Cl₂ (0.75 mL) was added under argon atmosphere which resulted in yellow reaction mixture. After 10 minutes, a solution of (S)-5-bromopent-1-en-4-yn-3-ol (0.114 g, 0.71 mmol) in CH₂Cl₂ (1.1 mL) was added dropwise over 5 minutes. A few crystals of NH₂OH.HCl were added as necessary whenever solution turned blue or green. After 20 minutes, analysis of the reaction mixture by GC/MS showed that there was still some (R)-3-methoxydodec-1-yne present. At 50 minutes, added additional (S)-5-bromopent-1-en-4-yn-3-ol (0.075 g, 0.47 mmol) in CH₂Cl₂ (0.5 mL). Reaction was stirred an additional 1 hour and then quenched with water, extracted 3 times with CH₂Cl₂, dried over Na₂SO₄, and concentrated under reduced pressure. Immediate purification by flash chromatography on silica gel (hexanes—10% EtOAc/hexanes) afforded compound 58 (0.089 g, 62.1%, brown oil).

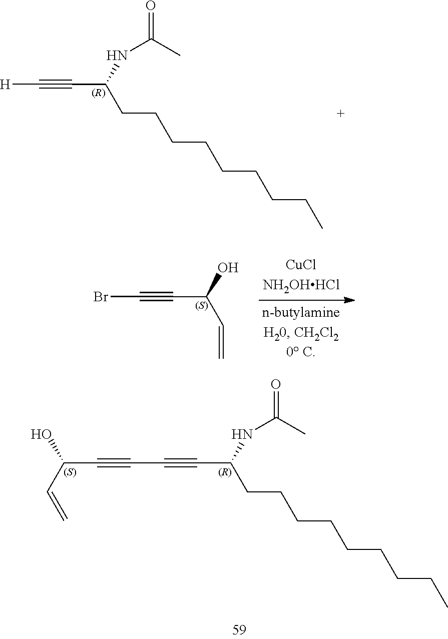

### Example 20

### Synthesis of N-((3S,8R)-3-hydroxyheptadeca-1-en-4,6-diyn-8-yl)acetamide (compound 59)

Copper (I) chloride (0.007 g, 0.07 mmol) was added to a stirred 30% solution (1.0 mL) of n-butylamine in distilled water at 0° C. which resulted in a deep blue solution. A few crystals of NH₂OH.HCl were added until solution was colorless. A solution of (R)—N-(dodec-1-yn-3-yl)acetamide (0.074 g, 0.33 mmol) in CH₂Cl₂ (0.5 mL) was added under argon atmosphere which resulted in yellow reaction mixture. After 10 minutes, a solution of (S)-5-bromopent-1-en-4-yn-3-ol (0.109 g, 0.68 mmol) in CH₂Cl₂ (1.0 mL) was added dropwise over 5 minutes. A few crystals of NH₂OH.HCl were added as necessary whenever solution turned blue or green. After 45 minutes, reaction was quenched with water, extracted 3 times with CH₂Cl₂, dried over Na₂SO₄, and concentrated under reduced pressure. Immediate purification by flash chromatography on silica gel (hexanes—40% EtOAc/hexanes) afforded compound 59 (0.093 g, 92.5%, yellow oil).

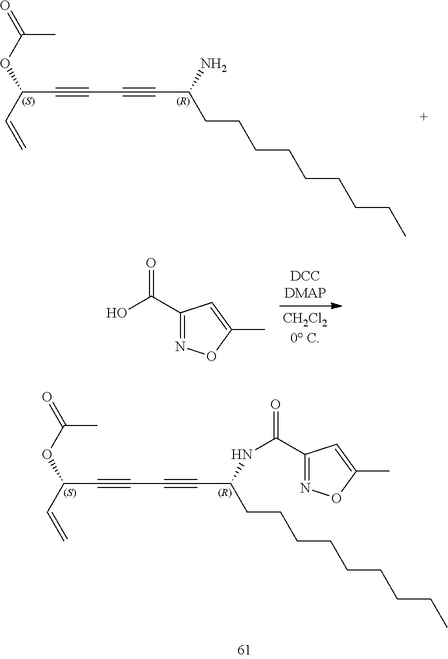

### Example 21

### Synthesis of (3S,8R)-8-(5-methylisoxazole-3-carboxamido)heptadeca-1-en-4,6-diyn-3-yl acetate (compound 61)

To a suspension of 5-methylisoxazole-3-carboxylic acid (0.052 g, 0.41 mmol) in CH₂Cl₂ (1.6 mL) at 0° C. under Ar was added DCC (0.062 g, 0.30 mmol). Then a solution of compound 45 (0.080 g, 0.26 mmol) in CH₂Cl₂ (1.0 mL) was added followed by addition of DMAP (5.5 mg, 0.045 mmol). After 17 hours, reaction was quenched with saturated NH₄Cl, extracted with CH₂Cl₂, washed with water and brine, dried over Na₂SO₄, and concentrated under reduced pressure. Purification by flash chromatography on silica gel (hexanes—15% EtOAc/hexanes) afforded compound 61 (23.1 mg, 21.2%) as a yellow oil.

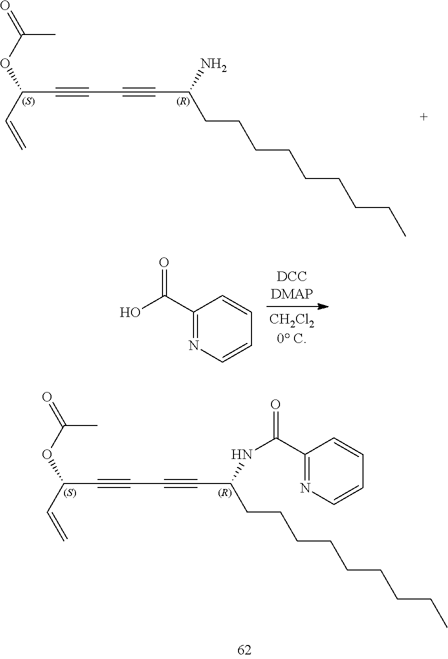

### Example 22

### Synthesis of (3S,8R)-8-(picolinamido)heptadeca-1-en-4,6-diyn-3-yl acetate (compound 62)

To a suspension of pyridine-2-carboxylic acid (0.031 g, 0.25 mmol) in CH₂Cl₂ (1.0 mL) at 0° C. under Ar was added DCC (0.054 g, 0.26 mmol). Then a solution of compound 45 (0.060 g, 0.20 mmol) in CH₂Cl₂ (1.0 mL) was added followed by addition of DMAP (3.9 mg, 0.032 mmol). After 2 hours and 10 minutes, reaction was quenched with saturated NH₄Cl, diluted with Et₂O, extracted three times with Et₂O, washed with water, saturated NaHCO₃, and brine, dried over Na₂SO₄, and concentrated under reduced pressure. Purification by flash chromatography on silica gel (hexanes—20% EtOAc/hexanes) afforded compound 62 (0.061 g, 75.5%) as a yellow oil.

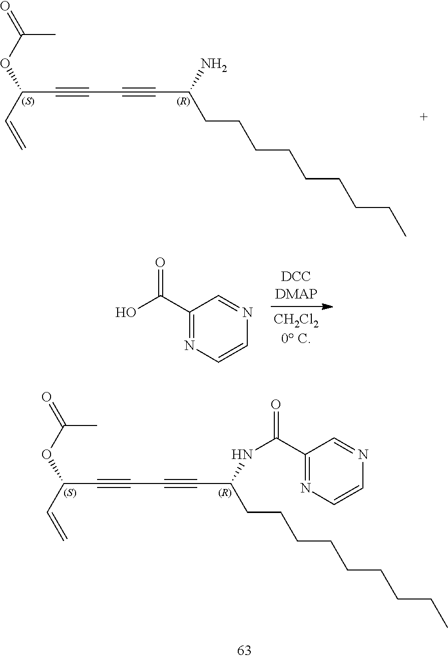

### Example 23

### Synthesis of (3S,8R)-8-(pyrazine-2-carboxamido)heptadeca-1-en-4,6-diyn-3-yl acetate (compound 63)

To a suspension of pyrazinecarboxylic acid (0.033 g, 0.27 mmol) in CH₂Cl₂ (1.0 mL) at 0° C. under Ar was added DCC (0.053 g, 0.26 mmol). Then a solution of compound 45 (0.062 g, 0.20 mmol) in CH₂Cl₂ (1.0 mL) was added followed by addition of DMAP (5.2 mg, 0.043 mmol). After 1 hour and 30 minutes, reaction was quenched with saturated NH₄Cl, diluted with Et₂O, extracted three times with Et₂O, washed with water, saturated NaHCO₃, and brine, dried over Na₂SO₄, and concentrated under reduced pressure. Purification by flash chromatography on silica gel (hexanes—25% EtOAc/hexanes) afforded compound 63 (0.057 g, 68.1%) as a yellow oil.

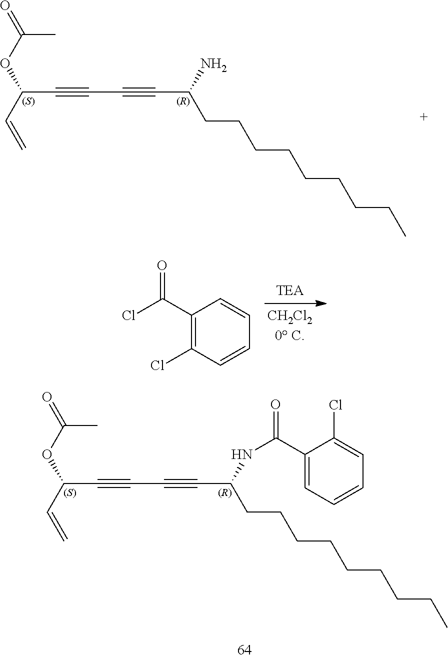

### Example 24

### Synthesis of (3S,8R)-8-(2-chlorobenzamido)heptadeca-1-en-4,6-diyn-3-yl acetate (compound 64)

To a solution of compound 45 (0.060 g, 0.20 mmol) in CH₂Cl₂ (2.0 mL) at 0° C. under Ar atmosphere was added triethylamine (0.04 mL, 0.29 mmol) followed by a solution of 2-chlorobenzoyl chloride (0.03 mL, 0.24 mmol) in CH₂Cl₂ (1 mL). Reaction mixture was stirred at 0° C. for 4 hours and 30 minutes. Reaction was then quenched with saturated NH₄Cl, diluted with Et₂O, extracted three times with Et₂O, washed with water, saturated NaHCO₃, and brine, dried over Na₂SO₄, and concentrated under reduced pressure. Purification by flash chromatography on silica gel (hexanes—15% EtOAc/hexanes) afforded compound 64 but was contaminated per NMR. Product was diluted in Et₂O, washed ten times with saturated NaHCO₃, washed two times with brine, dried over Na₂SO₄ and concentrated under reduced pressure to afford compound 64 (0.043 g, 49.5%) as a yellow oil.

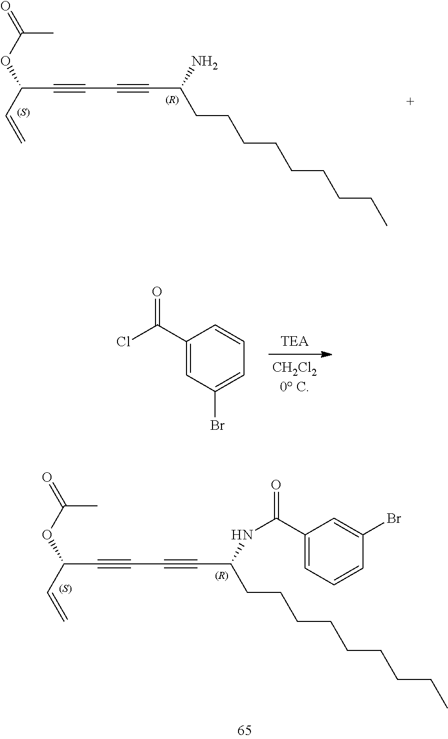

### Example 25

### Synthesis of (3S,8R)-8-(3-bromobenzamido)heptadeca-1-en-4,6-diyn-3-yl acetate (compound 65)

To a solution of compound 45 (0.057 g, 0.20 mmol) in CH₂Cl₂ (1.0 mL) at 0° C. under Ar atmosphere was added triethylamine (0.04 mL, 0.29 mmol) followed by a solution of 3-bromobenzoyl chloride (0.03 mL, 0.23 mmol) in CH₂Cl₂ (1 mL). Reaction mixture was stirred at 0° C. for 1 hour and 10 minutes. Reaction was then quenched with saturated NH₄Cl, diluted with Et₂0, extracted three times with Et₂O, washed with water, saturated NaHCO₃, and brine, dried over Na₂SO₄, and concentrated under reduced pressure. Purification by flash chromatography on silica gel (hexanes—15% EtOAc/hexanes) afforded compound 65 (0.051 g, 53.2%) as a yellow-orange oil.

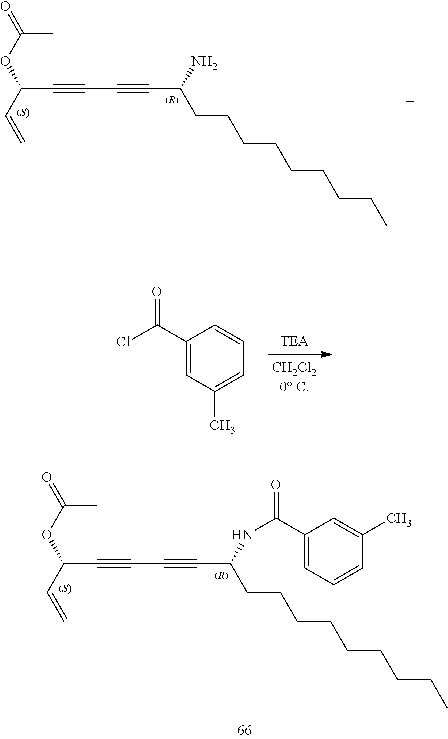

### Example 26

### Synthesis of (3S,8R)-8-(3-methylbenzamido)heptadeca-1-en-4,6-diyn-3-yl acetate (compound 66)

To a solution of compound 45 (0.059 g, 0.20 mmol) in CH₂Cl₂ (1.0 mL) at 0° C. under Ar atmosphere was added triethylamine (0.04 mL, 0.29 mmol) followed by a solution of m-toluoylbenzoyl chloride (0.03 mL, 0.23 mmol) in CH₂Cl₂ (1 mL). Reaction mixture was stirred at 0° C. for 1 hour and 40 minutes. Reaction was then quenched with saturated NH₄Cl, diluted with Et₂O, extracted three times with Et₂O, washed with water, saturated NaHCO₃, and brine, dried over Na₂SO₄, and concentrated under reduced pressure. Purification by flash chromatography on silica gel (hexanes—15% EtOAc/hexanes) afforded compound 66 but was contaminated with m-toluoylcarboxylic acid. Product was diluted with Et₂O, washed ten times with saturated NaHCO₃, washed twice with brine, dried over Na₂SO₄, and concentrated under reduced pressure to afford compound 66 (0.035 g, 42.7%) as a yellow solid.

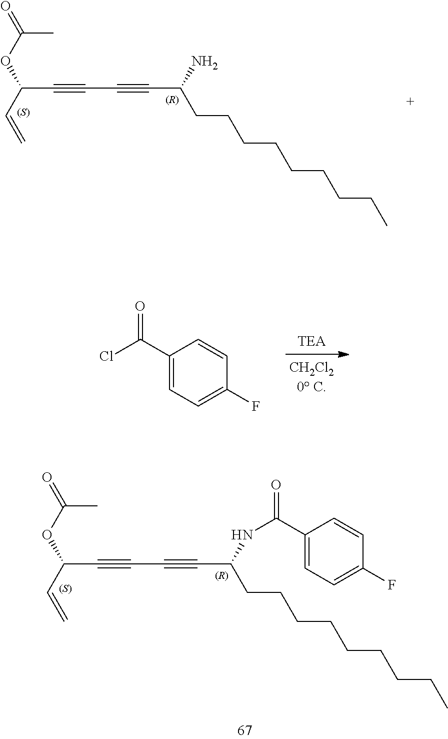

### Example 27

### Synthesis of (3S,8R)-8-(4-fluorobenzamido)heptadeca-1-en-4,6-diyn-3-yl acetate (compound 67)

To a solution of compound 45 (0.059 g, 0.20 mmol) in CH₂Cl₂ (2.0 mL) at 0° C. under Ar atmosphere was added triethylamine (0.04 mL, 0.29 mmol) followed by a solution of 4-fluorobenzoyl chloride (0.03 mL, 0.25 mmol) in CH₂Cl₂ (1 mL). Reaction mixture was stirred at 0° C. for 1 hour and 40 minutes. Reaction mixture was then quenched with water, extracted three times with CH₂Cl₂, washed with water and saturated NaHCO₃, dried over Na₂SO₄, and concentrated under reduced pressure. Purification by flash chromatography on silica gel (hexanes—10% EtOAc/hexanes) afforded compound 67 (0.057 g, 68.6%, light yellow oil).

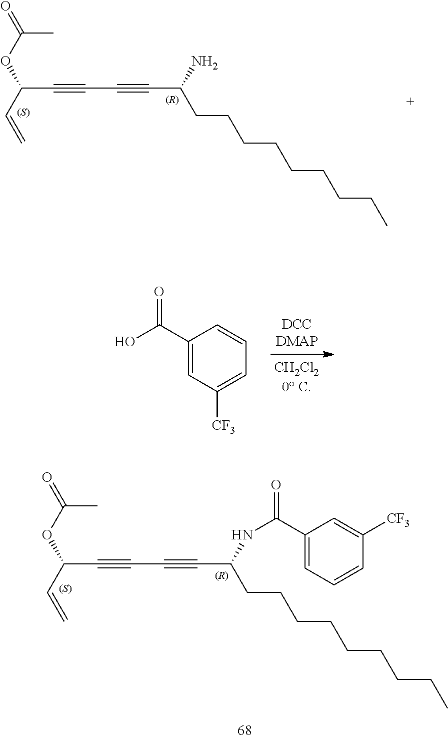

### Example 28

### Synthesis of (3S,8R)-8-(3-(trifluoromethyl)benzamido)heptadeca-1-en-4,6-diyn-3-yl acetate (compound 68)

To a suspension of 3-(trifluoromethyl)benzoic acid (0.045 g, 0.24 mmol) in CH₂Cl₂ (1.0 mL) at 0° C. under Ar was added DCC (0.051 g, 0.25 mmol). Then a solution of compound 45 (0.060 g, 0.20 mmol) in CH₂Cl₂ (1.0 mL) was added followed by addition of DMAP (5.8 mg, 0.047 mmol). After 1 hour and 20 minutes, reaction was quenched with saturated NH₄Cl, diluted with Et₂O, extracted three times with Et₂O, washed with water, saturated NaHCO₃, and brine, dried over Na₂SO₄, and concentrated under reduced pressure. Purification by flash chromatography on silica gel (hexanes—20% EtOAc/hexanes) afforded compound 68 (0.058 g, 62.2%) as a yellow oil.

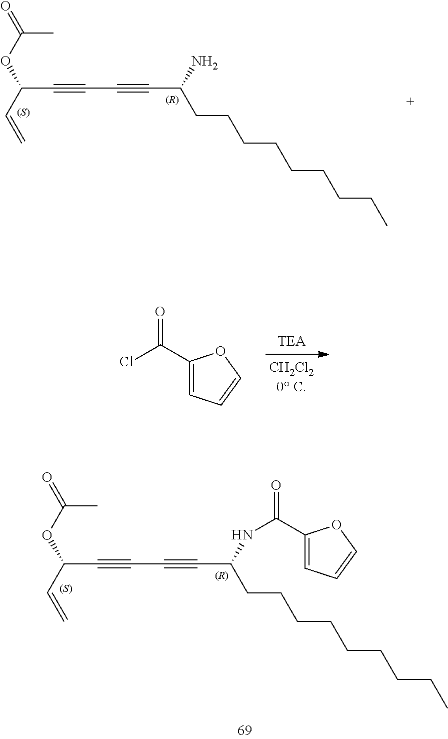

### Example 29

### Synthesis of (3S,8R)-8-(furan-2-carboxamido)heptadeca-1-en-4,6-diyn-3-yl acetate (compound 69)

To a solution of compound 45 (0.060 g, 0.20 mmol) in CH₂Cl₂ (1.0 mL) at 0° C. under Ar atmosphere was added triethylamine (0.04 mL, 0.29 mmol) followed by a solution of 2-furoyl chloride (0.02 mL, 0.21 mmol) in CH₂Cl₂ (1 mL). Reaction mixture was stirred at 0° C. for 1 hour and 5 minutes. Reaction was then quenched with saturated NH₄Cl, diluted with Et₂O, extracted three times with Et₂O, washed with water, saturated NaHCO₃, and brine, dried over Na₂SO₄, and concentrated under reduced pressure. Purification by flash chromatography on silica gel (hexanes—15% EtOAc/hexanes) afforded compound 69 but it was contaminated per GC/MS analysis. Product was diluted with Et₂O and washed 10 times with saturated NaHCO₃. However, it was still contaminated per GC/MS analysis. It was again diluted in Et₂O and stirred rapidly with 6M NaOH (aq.). It was then extracted three times with Et₂O, washed with water, saturated NaHCO₃, and brine, dried over Na₂SO₄, and concentrated under reduced pressure to afford purified compound 69 (0.042 g, 53.5%) as a yellow oil.

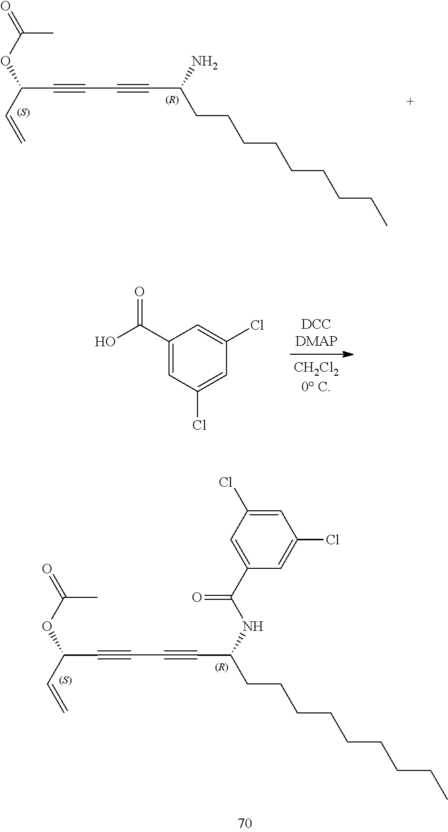

### Example 30

### Synthesis of (3S,8R)-8-(3,5-dichlorobenzamido)heptadeca-1-en-4,6-diyn-3-yl acetate (compound 70)

To a suspension of 3,5-dichlorobenzoic acid (0.044 g, 0.23 mmol) in CH₂Cl₂ (1.0 mL) at 0° C. under Ar was added DCC (0.048 g, 0.23 mmol). Then a solution of compound 45 (0.061 g, 0.20 mmol) in CH₂Cl₂ (1.0 mL) was added followed by addition of DMAP (3.7 mg, 0.030 mmol). After 2 hours and 40 minutes, reaction was quenched with saturated NH₄Cl, diluted with Et₂O, extracted three times with Et₂O, washed two times with water, washed 5 times with 1M NaOH, washed 2 times with brine, dried over Na₂SO₄, and concentrated under reduced pressure. Purification by flash chromatography on silica gel (hexanes—10% EtOAc/hexanes) afforded compound 70 (0.037 g, 39.1%) as a yellow oil.

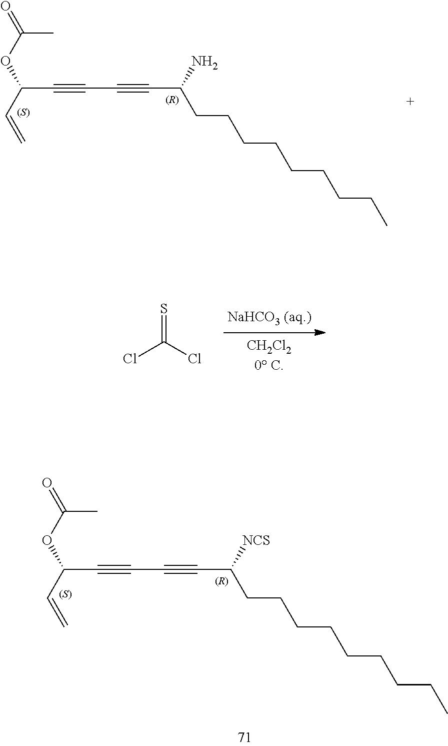

### Example 31

### Synthesis of (3S,8R)-8-isothiocyanatoheptadeca-1-en-4,6-diyn-3-yl acetate (compound 71)

To a solution of compound 45 (0.072 g, 0.24 mmol) in CH₂Cl₂ (0.9 mL) was added saturated aqueous NaHCO₃ (0.9 mL). Reaction was then cooled to 0° C. under Ar atmosphere and thiophosgene 0.035 mL, 0.46 mmol) was added. Reaction mixture was stirred at 0° C. for 1 hour and 20 minutes. Reaction was then diluted with CH₂Cl₂ and saturated aqueous NaHCO₃, extracted three times with CH₂Cl₂, washed with brine, dried over Na₂SO₄, and concentrated under reduced pressure. Purification by flash chromatography on silica gel (hexanes—5% EtOAc/hexanes) afforded compound 71 (0.029 g, 35.3%) as a light brown oil.

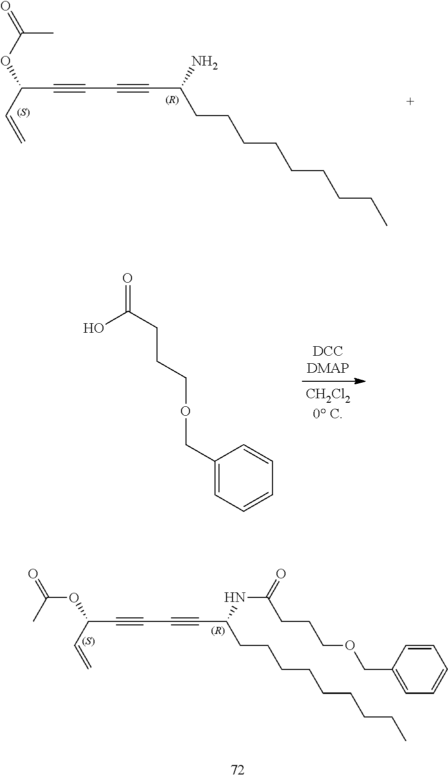

### Example 32

### Synthesis of (3S,8R)-8-(4-(benzyloxy)butanamido)heptadeca-1-en-4,6-diyn-3-yl acetate (compound 72)

To a suspension of 4-benzyloxybutyric acid (0.069 g, 0.36 mmol) in CH₂Cl₂ (1.0 mL) at 0° C. under Ar was added DCC (0.052 g, 0.25 mmol). Then a solution of compound 45 (0.060 g, 0.20 mmol) in CH₂Cl₂ (1.0 mL) was added followed by addition of DMAP (4.3 mg, 0.035 mmol). After 3 hours, reaction was quenched with saturated NH₄Cl, diluted with Et₂O, extracted three times with Et₂O, washed two times with water, washed 5 times with saturated NaHCO₃, washed 2 times with brine, dried over Na₂SO₄, and concentrated under reduced pressure. Purification by flash chromatography on silica gel (hexanes—25% EtOAc/hexanes) afforded compound 72 (0.067 g, 70.9%) as a yellow oil.

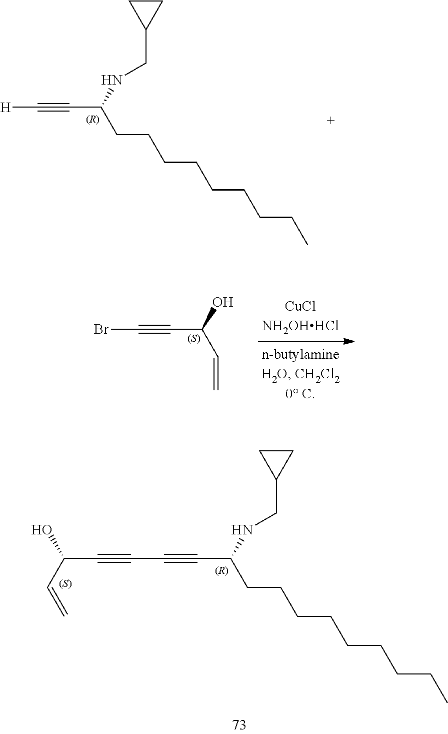

### Example 33

### Synthesis of (3S,8R)-8-((cyclopropylmethyl)amino)heptadeca-1-en-4,6-diyn-3-ol (compound 73)

Copper (I) chloride (0.007 g, 0.07 mmol) was added to a stirred 30% solution (1.2 mL) of n-butylamine in distilled water at 0° C. which resulted in a deep blue solution. A few crystals of NH₂OH.HCl were added until solution was colorless. A solution of (R)—N-(cyclopropylmethyl)dodec-1-yn-3-amine (0.090 g, 0.38 mmol) in CH₂Cl₂ (0.6 mL) was added under argon atmosphere which resulted in yellow reaction mixture. After 10 minutes, a solution of (S)-5-bromopent-1-en-4-yn-3-ol (0.102 g, 0.64 mmol) in CH₂Cl₂ (0.9 mL) was added dropwise over 5 minutes. A few crystals of NH₂OH.HCl were added as necessary whenever solution turned blue or green. After 30 minutes, reaction was quenched with water, extracted 3 times with CH₂Cl₂, dried over Na₂SO₄, and concentrated under reduced pressure. Immediate purification by flash chromatography on silica gel (hexanes—30% EtOAc/hexanes) afforded compound 73 but it was contaminated with residual (R)—N-(cyclopropylmethyl)dodec-1-yn-3-amine. A second round of flash chromatography on silica gel (Hexanes 25% EtOAc/hexanes) afforded purified compound 73 (0.015 g, 12.6%, brown oil).

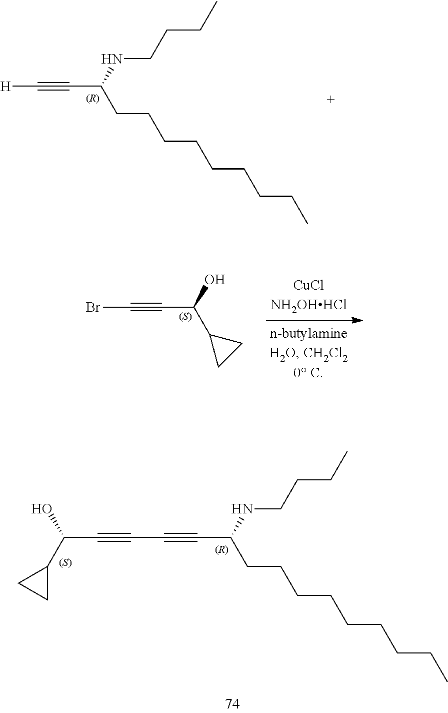

### Example 34

### Synthesis of (1S,6R)-6-(butylamino)-1-cyclopropylpentadeca-2,4-diyn-1-ol (compound 74)

Copper (I) chloride (0.007 g, 0.07 mmol) was added to a stirred 30% solution (1.25 mL) of n-butylamine in distilled water at 0° C. which resulted in a deep blue solution. A few crystals of NH₂OH.HCl were added until solution was colorless. A solution of (R)—N-butyldodec-1-yn-3-amine (0.104 g, 0.44 mmol) in CH₂Cl₂ (0.63 mL) was added under argon atmosphere which resulted in yellow reaction mixture. After 10 minutes, a solution of (S)-3-bromo-1-cyclopropylprop-2-yn-1-ol (0.135 g, 0.77 mmol) in CH₂Cl₂ (1.1 mL) was added dropwise over 5 minutes. A few crystals of NH₂OH.HCl were added as necessary whenever solution turned blue or green. After 1 hour and 10 minutes, reaction was quenched with water, extracted 3 times with CH₂Cl₂, dried over Na₂SO₄, and concentrated under reduced pressure. Immediate purification by flash chromatography on silica gel (hexanes—20% EtOAc/hexanes) afforded compound 74 (0.058 g, 39.9%, yellow solid).

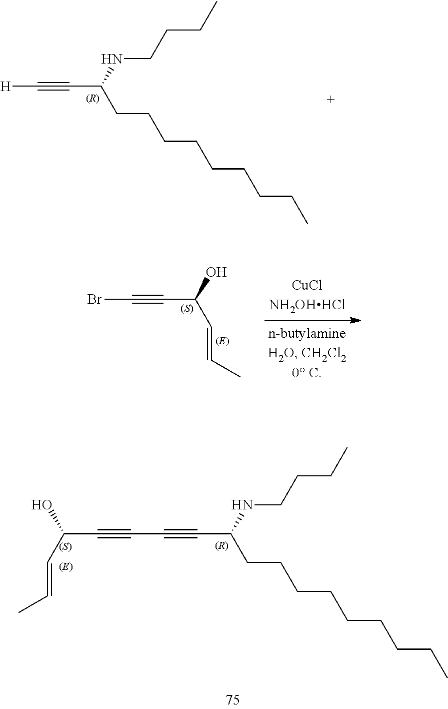

### Example 35

### Synthesis of (4S,9R,E)-9-(butylamino)octadeca-2-en-5,7-diyn-4-ol (compound 75)

Copper (I) chloride (0.010 g, 0.10 mmol) was added to a stirred 30% solution (1.25 mL) of n-butylamine in distilled water at 0° C. which resulted in a deep blue solution. A few crystals of NH₂OH.HCl were added until solution was colorless. A solution of (R)—N-butyldodec-1-yn-3-amine (0.100 g, 0.42 mmol) in CH₂Cl₂ (0.63 mL) was added under argon atmosphere which resulted in yellow reaction mixture. After 10 minutes, a solution of (S,E)-1-bromohex-4-en-1-yn-3-ol (0.133 g, 0.76 mmol) in CH₂Cl₂ (1.1 mL) was added dropwise over 5 minutes. A few crystals of NH₂OH.HCl were added as necessary whenever solution turned blue or green. After 3 hours, reaction was quenched with water, extracted 3 times with CH₂Cl₂, dried over Na₂SO₄, and concentrated under reduced pressure. Immediate purification by flash chromatography on silica gel (hexanes—20% EtOAc/hexanes) afforded compound 75 (0.049 g, 35.6%, tan solid).

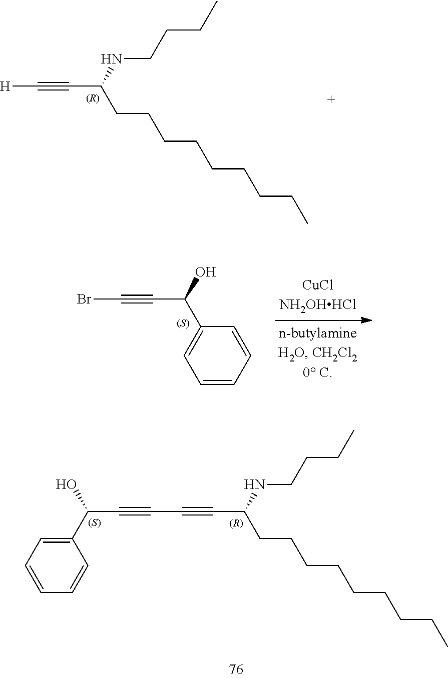

### Example 36

### Synthesis of (1S,6R)-6-(butylamino)-1-phenylpentadeca-2,4-diyn-1-ol (compound 76)

Copper (I) chloride (0.008 g, 0.08 mmol) was added to a stirred 30% solution (1.25 mL) of n-butylamine in distilled water at 0° C. which resulted in a deep blue solution. A few crystals of NH₂OH.HCl were added until solution was colorless. A solution of (R)—N-butyldodec-1-yn-3-amine (0.101 g, 0.43 mmol) in CH₂Cl₂ (0.63 mL) was added under argon atmosphere which resulted in yellow reaction mixture. After 10 minutes, a solution of (S)-3-bromo-1-phenylprop-2-yn-1-ol (0.159 g, 0.75 mmol) in CH₂Cl₂ (1.1 mL) was added dropwise over 5 minutes. A few crystals of NH₂OH.HCl were added as necessary whenever solution turned blue or green. After 50 minutes, reaction was quenched with water, extracted 3 times with CH₂Cl₂, dried over Na₂SO₄, and concentrated under reduced pressure. Immediate purification by flash chromatography on silica gel (hexanes—20% EtOAc/hexanes) afforded compound 76 (0.056 g, 35.7%, brownish yellow solid).

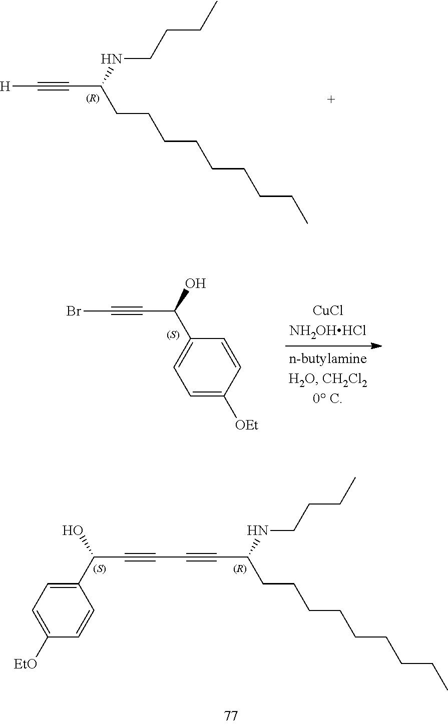

### Example 37

### Synthesis of (1S,6R)-6-(butylamino)-1-(4-ethoxyphenyl)pentadeca-2,4-diyn-1-ol (compound 77)

Copper (I) chloride (0.011 g, 0.11 mmol) was added to a stirred 30% solution (1.25 mL) of n-butylamine in distilled water at 0° C. which resulted in a deep blue solution. A few crystals of NH₂OH.HCl were added until solution was colorless. A solution of (R)—N-butyldodec-1-yn-3-amine (0.105 g, 0.44 mmol) in CH₂Cl₂ (0.63 mL) was added under argon atmosphere which resulted in yellow reaction mixture. After 10 minutes, a solution of (S)-3-bromo-1-(4-ethoxyphenyl)prop-2-yn-1-ol (0.194 g, 0.76 mmol) in CH₂Cl₂ (1.1 mL) was added dropwise over 5 minutes. A few crystals of NH₂OH.HCl were added as necessary whenever solution turned blue or green. After 2 hours and 20 minutes, reaction was quenched with water, extracted 3 times with CH₂Cl₂, dried over Na₂SO₄, and concentrated under reduced pressure. Immediate purification by flash chromatography on silica gel (hexanes—20% EtOAc/hexanes) afforded compound 77 (0.068 g, 37.3%, brownish yellow solid).

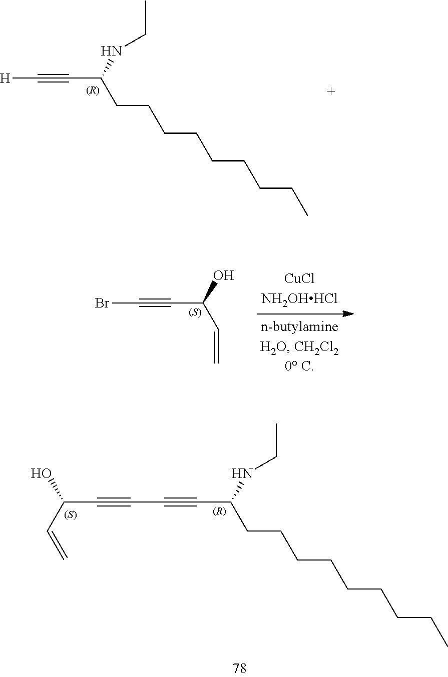

### Example 38

### Synthesis of (3S,8R)-8-(ethylamino)heptadeca-1-en-4,6-diyn-3-ol (compound 78)

Copper (I) chloride (0.007 g, 0.07 mmol) was added to a stirred 30% solution (1.2 mL) of n-butylamine in distilled water at 0° C. which resulted in a deep blue solution. A few crystals of NH₂OH.HCl were added until solution was colorless. A solution of (R)—N-ethyldodec-1-yn-3-amine (0.077 g, 0.37 mmol) in CH₂Cl₂ (0.6 mL) was added under argon atmosphere which resulted in yellow reaction mixture. After 10 minutes, a solution of (S)-5-bromopent-1-en-4-yn-3-ol (0.110 g, 0.68 mmol) in CH₂Cl₂ (1.0 mL) was added dropwise over 5 minutes. A few crystals of NH₂OH.HCl were added as necessary whenever solution turned blue or green. After 55 minutes, reaction was quenched with water, extracted 3 times with CH₂Cl₂, dried over Na₂SO₄, and concentrated under reduced pressure. Immediate purification by flash chromatography on silica gel (hexanes—40% EtOAc/hexanes) afforded compound 78 (0.032 g, 30.0%, brown solid).

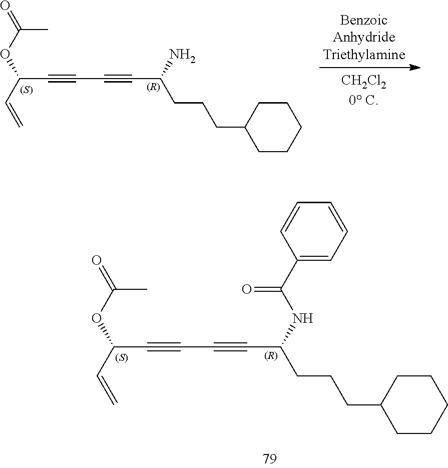

### Example 39

### Synthesis of (3S,8R)-8-benzamido-11-cyclohexylundeca-1-en-4,6-diyn-3-yl acetate (compound 79)

To a solution of (3S,8R)-8-amino-11-cyclohexylundeca-1-en-4,6-diyn-3-yl acetate (0.0465 g, 0.15 mmol) in CH₂Cl₂ (1.7 mL) at 0° C. under Ar was added benzoic anhydride (0.0454 g, 0.20 mmol) followed by triethylamine (0.030 mL, 0.22 mmol). Reaction mixture was stirred for 15 minutes at 0° C. and then allowed to warm to ambient temperature. After 1 hour and 30 minutes, reaction mixture was quenched with water, extracted three times with CH₂Cl₂, washed with saturated NaHCO₃, dried over Na₂SO₄, and concentrated under reduced pressure. Purification by flash chromatography on silica gel (hexanes—15% EtOAc/hexanes) afforded compound 79 (0.050 g, 79.6%, yellow oil).

### Example 40

### In Vitro Efficacy: Leishmaniasis

TABLE 1

| | IC₅₀ | IC₅₀ | |
|---|---|---|---|
| | Leishmania^{b} in | mammalian | |
| Compound | infected macro- | HepG2 | Selectivity |
| ID | phages [uM] | cells [uM] | Index^{c} |
| ^{a}(S,S)-1 | 1.28 | | |
| 1 | 0.6265 | 35 ± 3 | 60 |
| 2 | >10 | | |
| 3 | >10 | | |
| 20 | 0.197 | 57 ± 2 | 290 |
| 39 | 0.112 | | |
| 40 | 0.428 | | |
| 41 | 0.126 | | |
| 42 | 0.165 | | |
| 44 | 0.287 | | |
| 46 | 0.055 | 29 ± 1 | 530 |
| 47 | 0.035 | 486 ± 14 | 14000 |
| 48 | 0.034 | 342 ± 46 | 10000 |
| 49 | 0.061 | | |
| 50 | 0.013 | 38 ± 2 | 2900 |
| 55 | 0.003 | | |
| 56 | 0.005 | | |
| 57 | 0.010 | | |
| 58 | 0.011 | | |
| 59 | 0.018 | | |
| 61 | 0.077 | | |
| 62 | 0.089 | | |
| 63 | 0.042 | | |
| 64 | 0.109 | | |
| 65 | 0.184 | | |
| 66 | 0.120 | | |
| 67 | 0.072 | | |
| 68 | 0.191 | | |
| 69 | 0.050 | | |
| 70 | 0.252 | | |
| 71 | 21.65 | | |
| 72 | 0.529 | | |
| 74 | 0.722 | | |
| 75 | 0.031 | | |
| 76 | 0.113 | | |
| 77 | 0.443 | | |
| 78 | 0.030 | | |
| 79 | 0.027 | | |

^{a}Stereochemistry of Senn et al. product
^{b}Compounds (S,S)-1, 1, 2, and 3 were screened against Leishmania amazonensis; All
other compounds in table were screened against Leishmania donovani
^{c}Selectivity index = IC₅₀ for HepG2 cells/IC₅₀ for Leishmania species

An antileishmanial assay was performed as described in Bakunov et al. (Synthesis and Antiprotozoal Properties of Pentamidine Congeners Bearing the Benzofuran Motif, J. Med. Chem. 52(18):5673-5767, hereby incorporated by reference). Briefly, axenic amastigotes of L. donovani (WHO designation MHOM/SD/62/1S-CL2D) were adapted from promastigotes and grown in the amastigote medium described previously at 37° C. In a final volume of 60 μL, 6×10⁴ parasites were added to each well of a 96-well plate except for negative control wells. Standard and test compounds were added as appropriate using 2-fold dilutions to allow a range of concentrations to be tested. Plates were then incubated at 37° C. for 72 hours in a humidified environment containing 5% CO₂. The tetrazolium dye-based CellTiter reagent (Promega, Madison, Wis.) was used to assess parasite growth. Several hours after adding 12 μL of the CellTiter reagent to each well of the plate, absorbance readings were taken at 490 nm using a SpectraMax Plus 384 microplate reader (Molecular Devices, Sunnyvale, Calif.). SoftMax Pro software (Amersham Biosciences, Piscataway, N.J.) was used to calculate IC₅₀ values by employing the dose-response equation y=[(a−d)/(1b(x/c)b)]bd, where x=compound concentration, y=absorbance at 490 nm, a=upper asymptote, b=slope, c=IC50 value, and d=lower asymptote.

Using this assay, the natural product isolated by Senn et al. as well as the three diastereomers (compounds 1-3) were synthesized and analyzed for biological activity against Leishmania amazonensis which showed the (3S,8R) stereoisomer (compound 1) had the lowest IC₅₀ of 0.63 μM while the (3S,8S) stereoisomer (i.e., Senn compound) had an IC₅₀ of 1.28 μM (Table 1). The (3R,8R) and (3R,8S) stereoisomers (compounds 2 and 3, respectively) displayed no anti-Leishmanial activity at 10 μM indicating the stereochemistry plays a significant role in the anti-Leishmanial action of the compounds. Since compound 1 had the lowest IC₅₀, several analogues were synthesized with the same stereochemistry. Five of the analogues showed over an order of magnitude increase in anti-Leishmanial activity as compared with compound 1. Of these five analogues, compound 50 had the lowest IC₅₀ of 0.013 μM which represented a 48-fold increase in anti-Leishmanial activity as compared with compound 1. Compounds 46-49 showed increases in anti-Leishmanial activity ranging from 11-fold to 18-fold increases as compared with compound 1.

Several of these analogues were also evaluated for cytotoxicity against human liver (HepG2) cells. Of particular interest was that compounds 47 and 48 were approximately an order of magnitude less cytotoxic as compared to compound 1. More specifically, compound 1 had an IC₅₀ of 35±3 μM against the HepG2 cell line. Compounds 47 and 48 had IC₅₀ values of 486±14 μM and 342±46 μM, respectively against the HepG2 cell line. Compound 50 had an IC₅₀ value of 38±2 04 which is very similar to compound 1.

Comparing the cytotoxicity IC₅₀ values to the antileishmanial IC₅₀ values, the selectivity index (IC₅₀ for HepG2 assay/IC₅₀ for antileishmanial assay) for each compound was calculated. Due to their significantly better cytotoxicity IC₅₀ values, Compounds 47 and 48 showed the highest selectivity indices of 14,000 and 10,000, respectively. By comparison, compounds 1 and 50 had selectivity indices of 60 and 2900, respectively.

### Example 41

### In Vivo Efficacy: Leishmaniasis

TABLE 2

| Group | Treatment | Dose (i.p.) |
|---|---|---|
| A (untreated control) | PBS | |
| B (vehicle control) | PEG 400/Ethanol | |
| C (miltefosine) | Miltefosine | 10 mg/kg |
| D (compound 47) | Compound 47 | 10 mg/kg |
| E (compound 48) | Compound 48 | 10 mg/kg |
| F (compound 50) | Compound 50 | 10 mg/kg |

### Treatment Preparation:

The following protocols were used to prepare the various treatments described in Table 2:
a. Miltefosine (10 mg/kg stock solution in sterile PBS): 7.5 mg miltefosine was weighed and put in a eppendorf tube, then 0.75 mL of sterile PBS was added. After vortexing, a clear solution was formed. Make 1 mg/mL solution daily by diluting 100 μL of 10 mg/mL miltefosine solution in 900 μL sterile PBS.
b. Vehicle control: 5% (v/v) DMSO, 50% (v/v) PEG 400, 10% (v/v) Ethanol, and 35% (v/v) distilled water.
c. Compound 47 (2 mg/mL solution of compound 47 in PEG 400/Ethanol vehicle): 60 mg/kg compound 47 was diluted with DMSO to make 40 mg/kg solution. Dilute the above solution 1:20 with PEG 400/EtOH/water daily. After vortexing and warming up at 37° C. for 30 min., a clear solution formed.
d. Compound 48 (2 mg/mL solution of compound 48 in PEG 400/Ethanol vehicle): 6.8 mg of compound 48 was dissolved in 0.17 mL of DMSO to make a 40 mg/mL stock solution. After vortexing, a yellowish-brown solution was formed. Diluted the above solution 1:20 with PEG 400/EtOH/water daily. After vortexing a clear solution formed.
e. Compound 50 (2 mg/mL solution of compound 50 in PEG 400/Ethanol vehicle): 8.4 mg of compound 50 was dissolved in 0.21 mL of DMSO to make a 40 mg/mL stock solution. After vortexing, a dark yellowish-brown solution was formed. Diluted the above solution 1:20 with PEG 400/EtOH/water. After vortexing a clear solution formed.

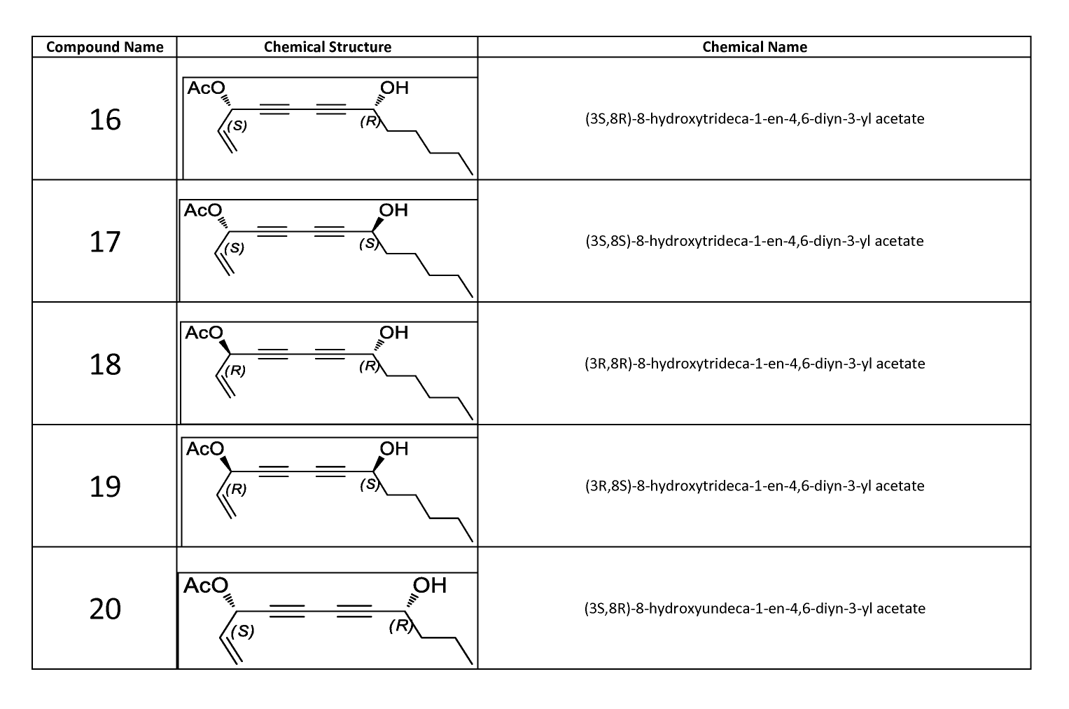

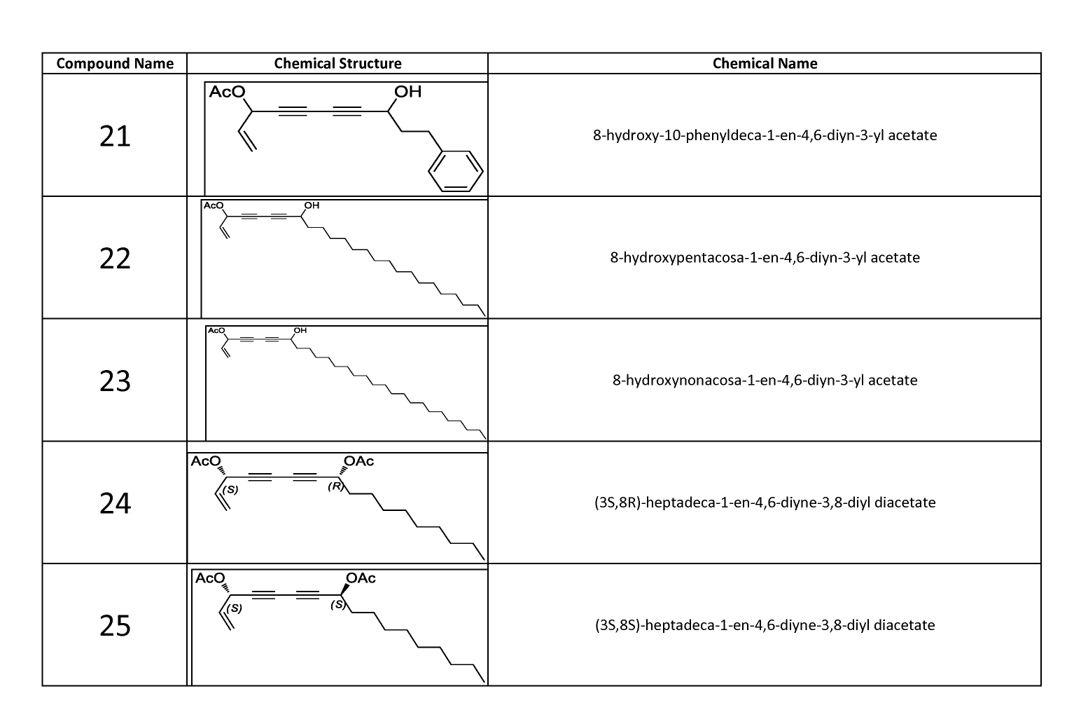

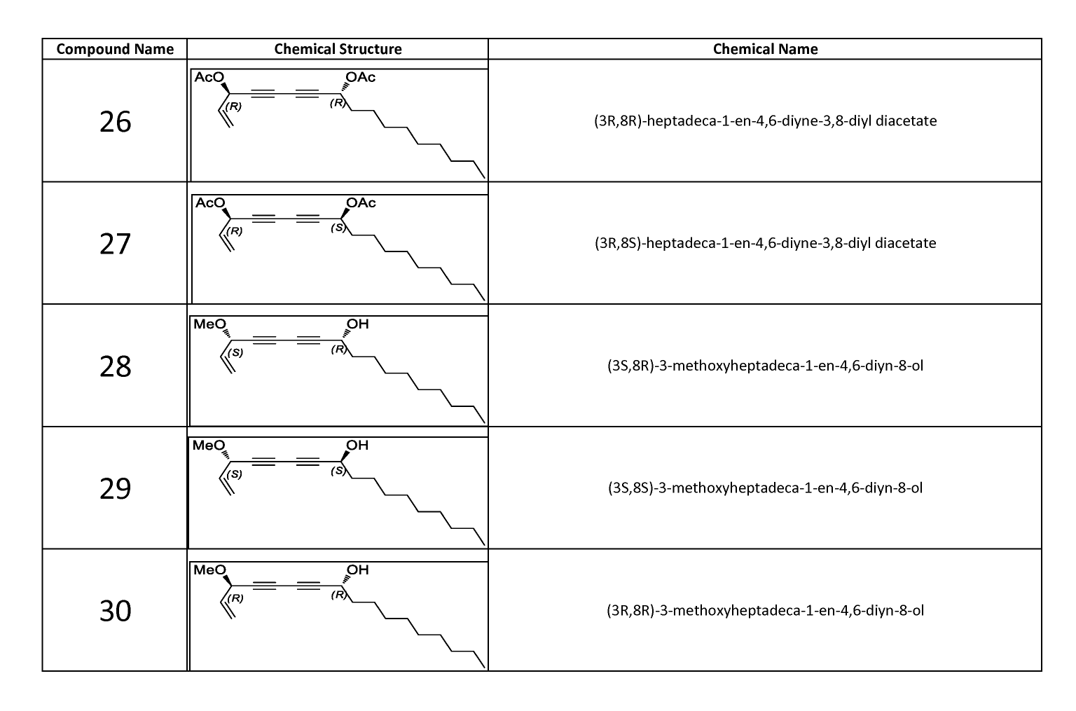

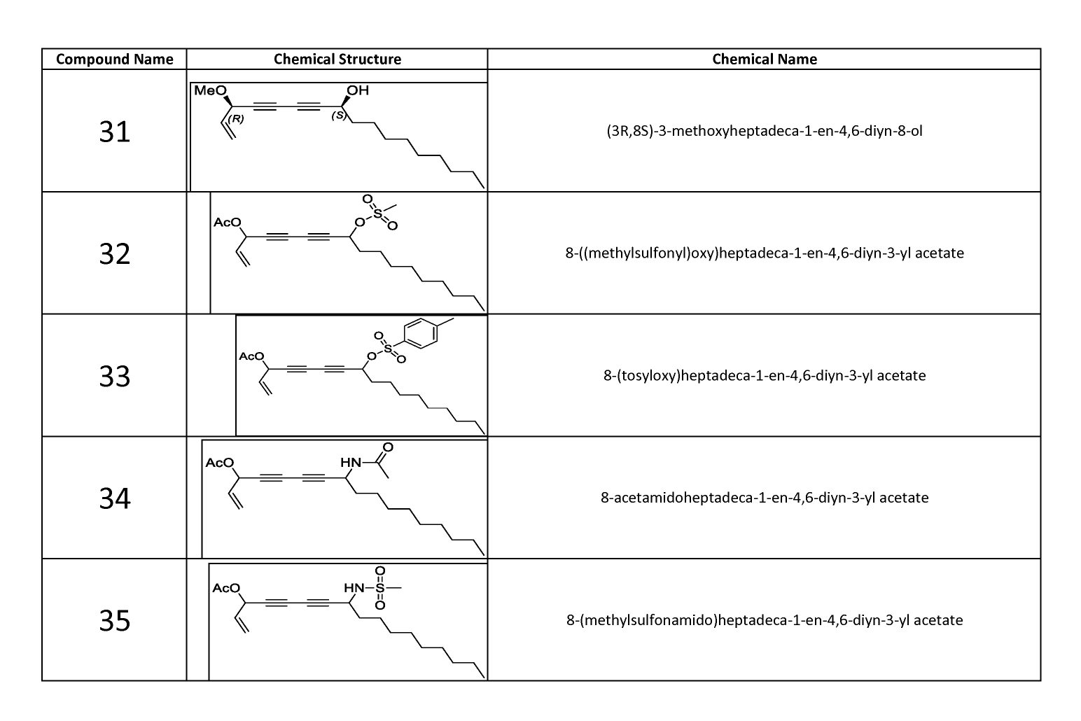

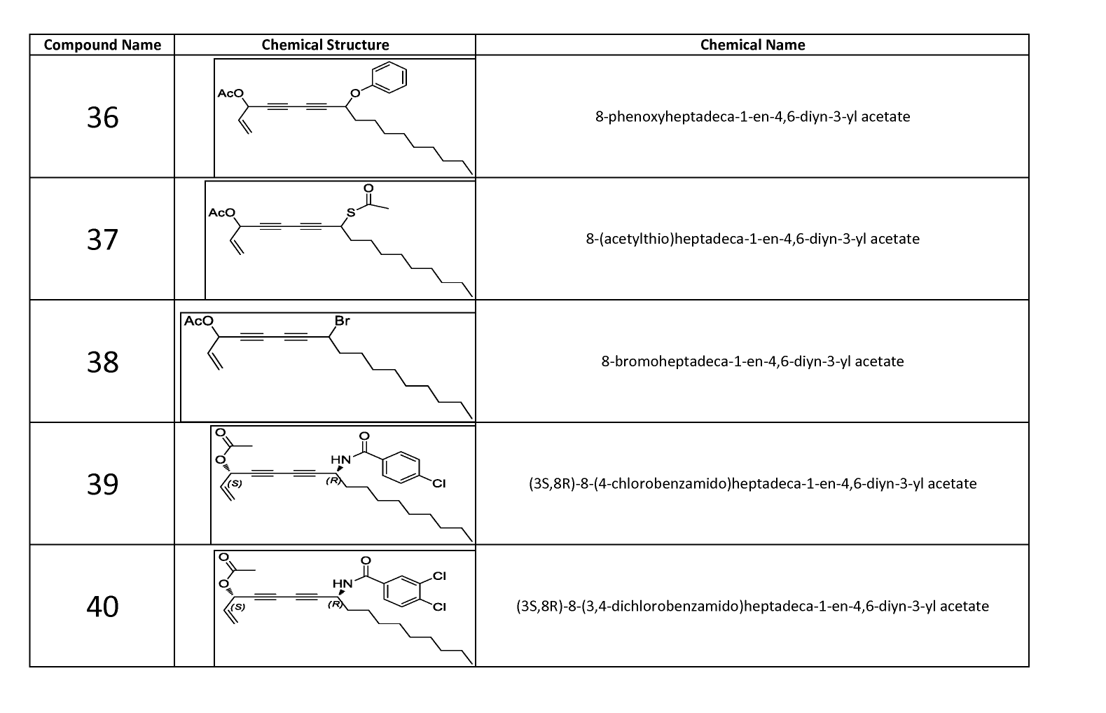

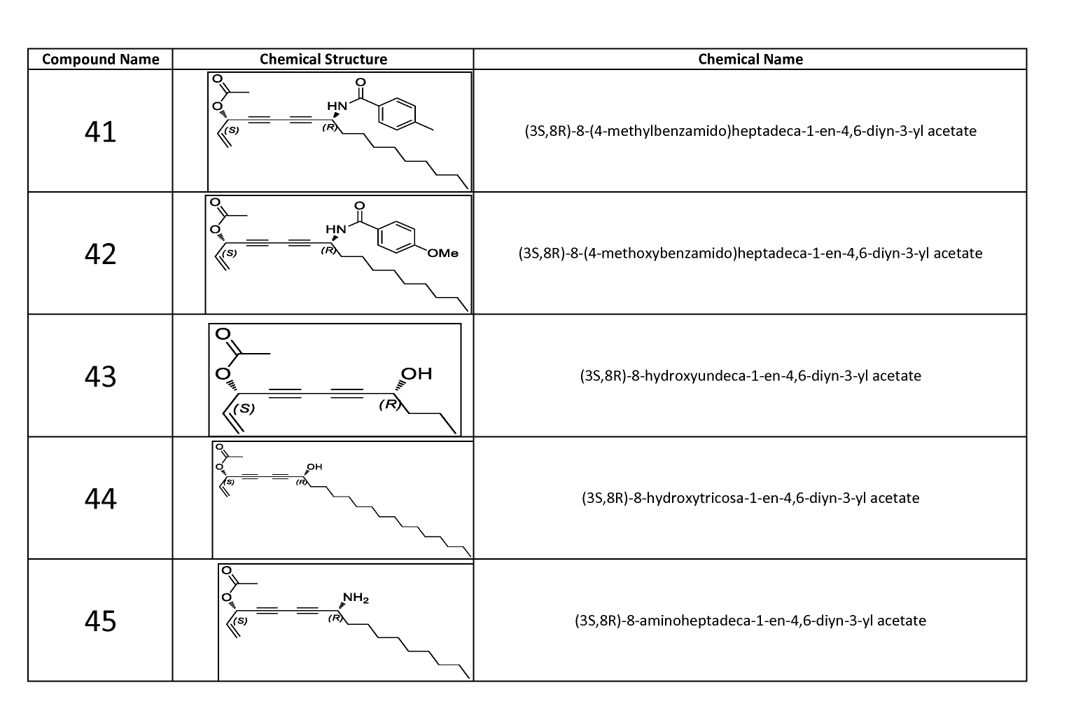

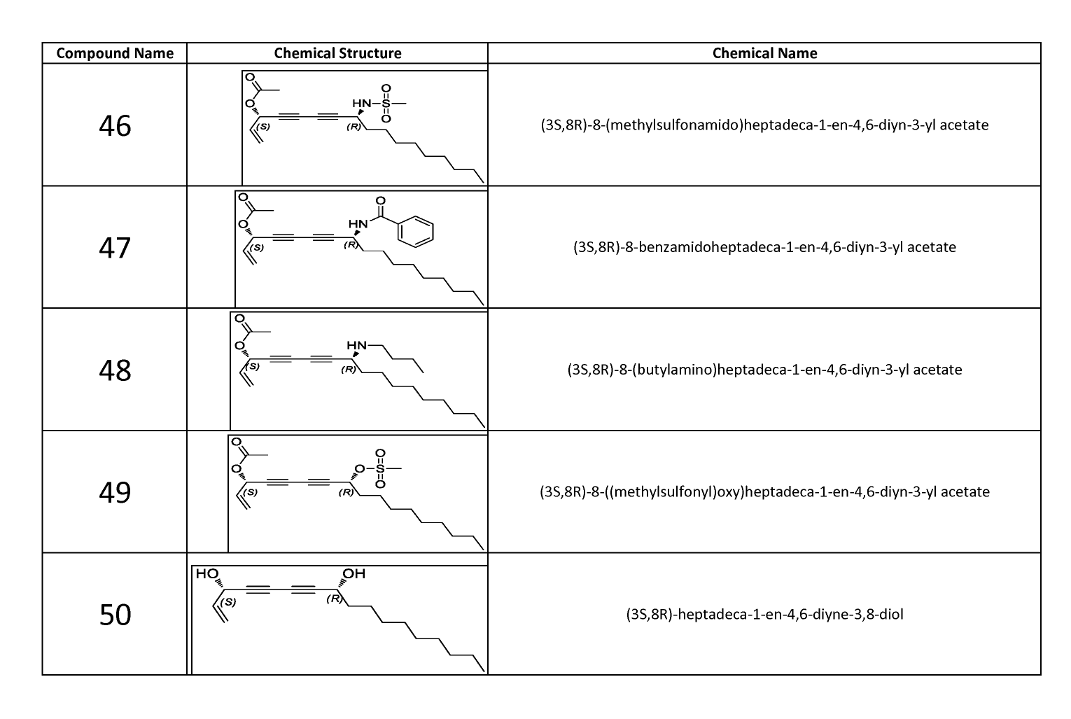

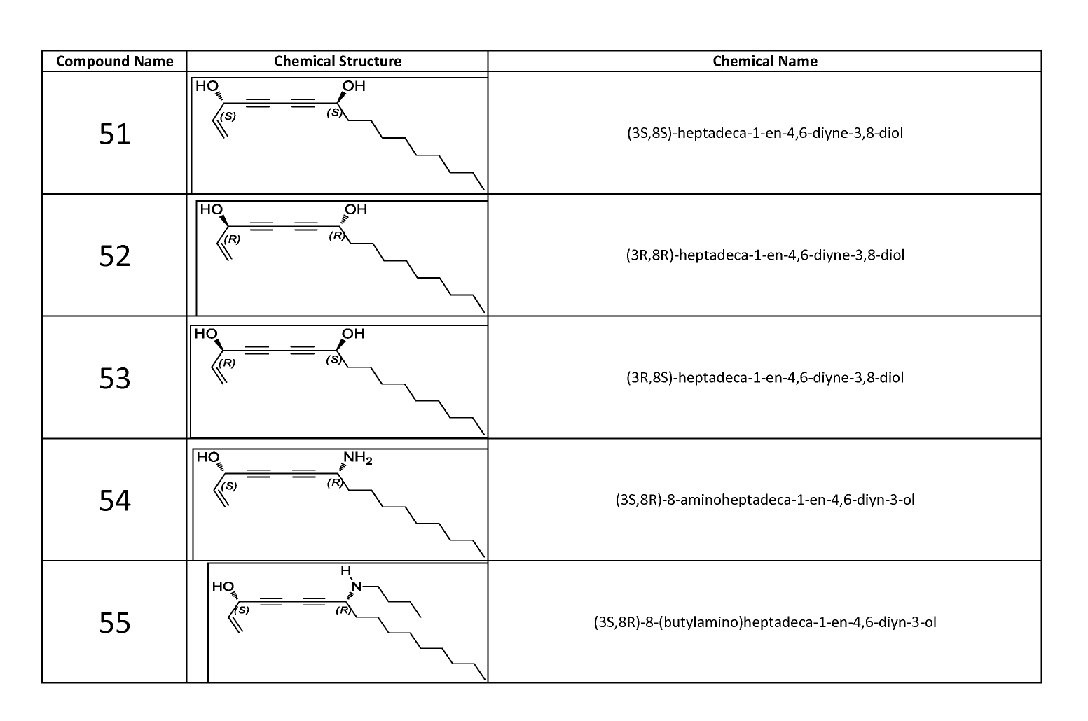

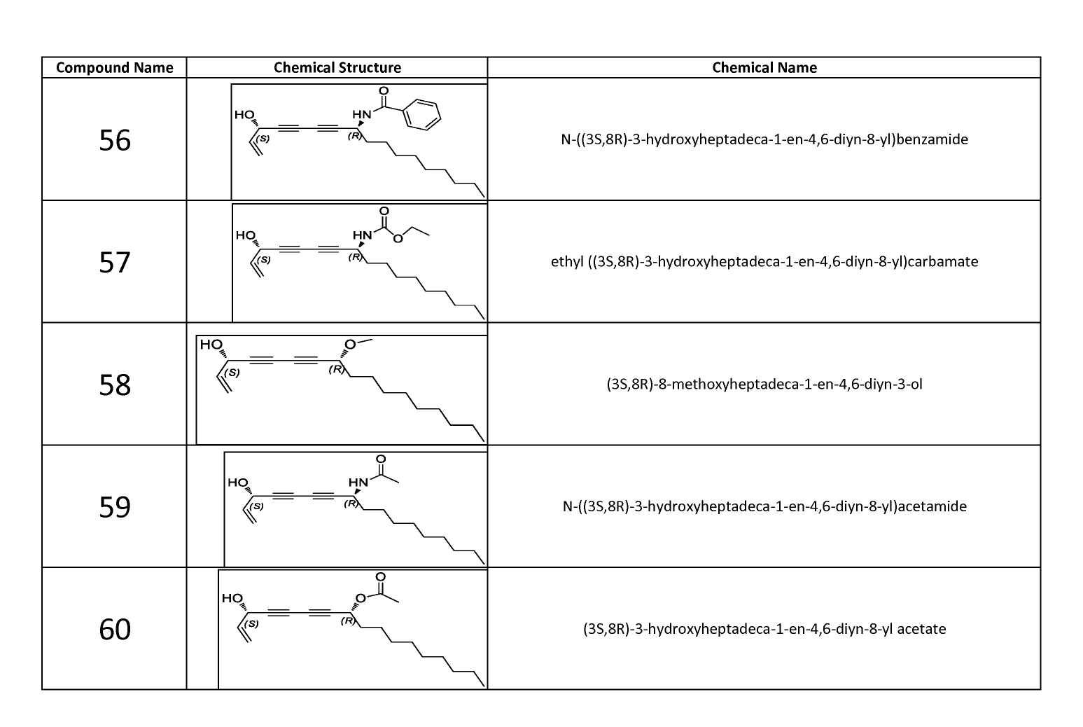

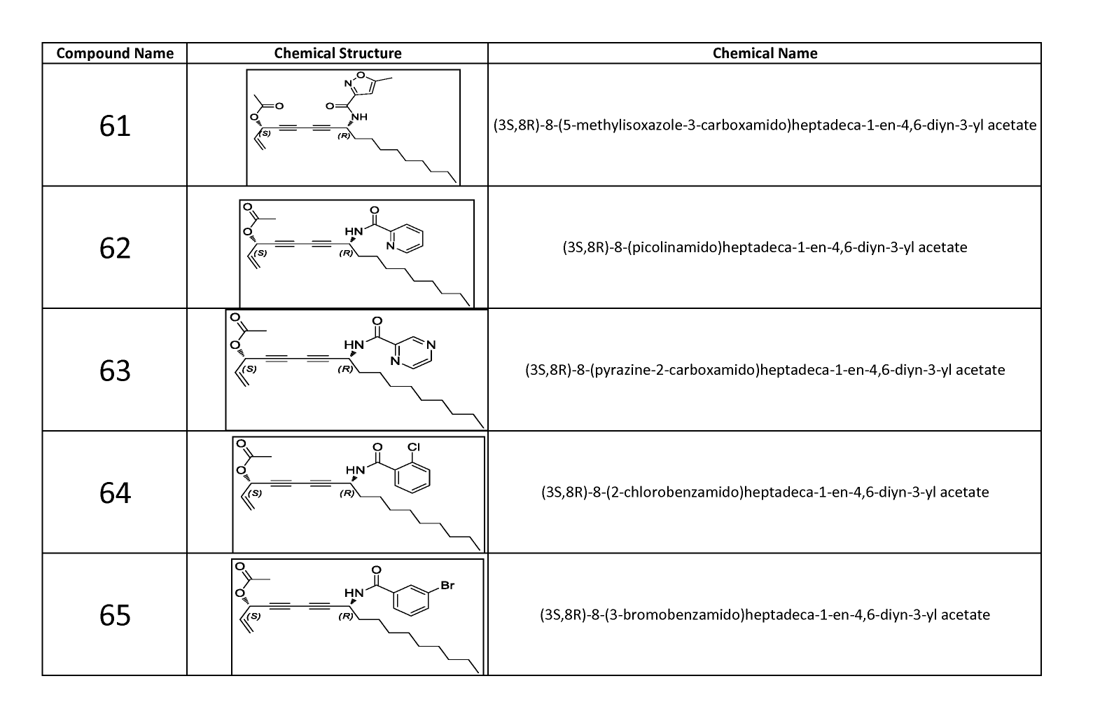

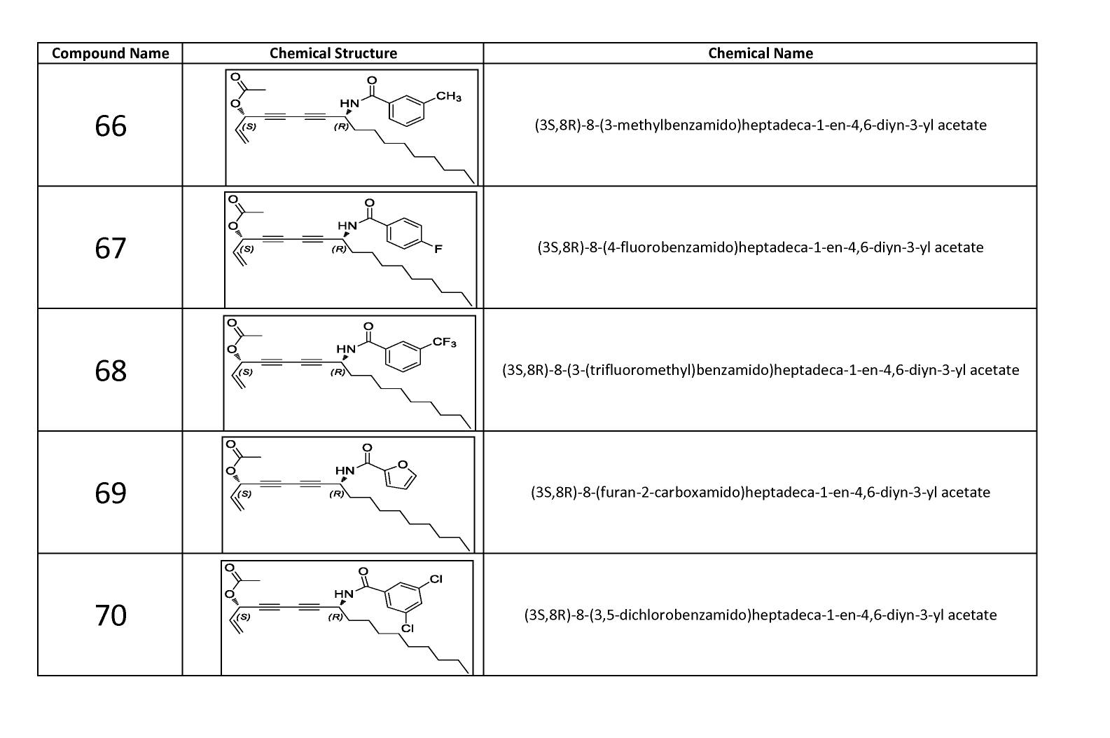

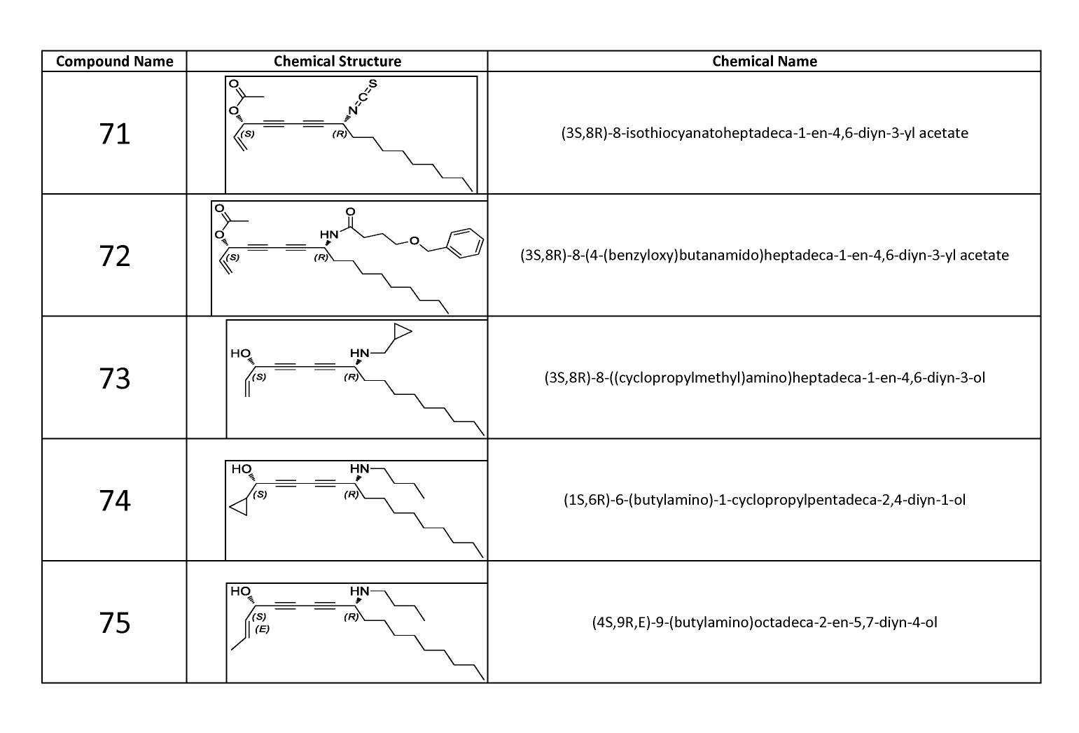

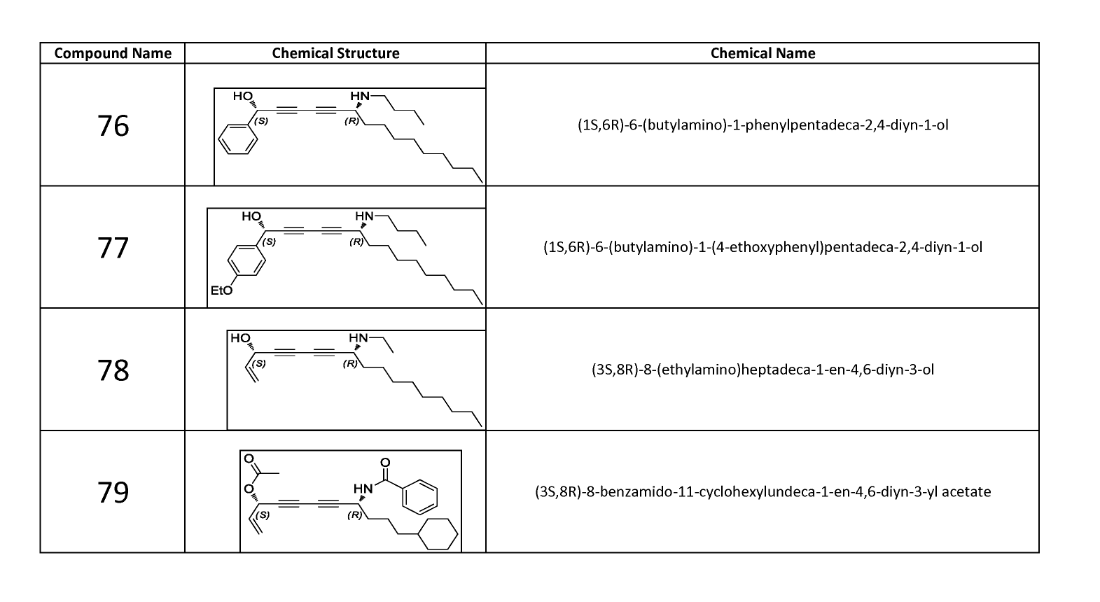

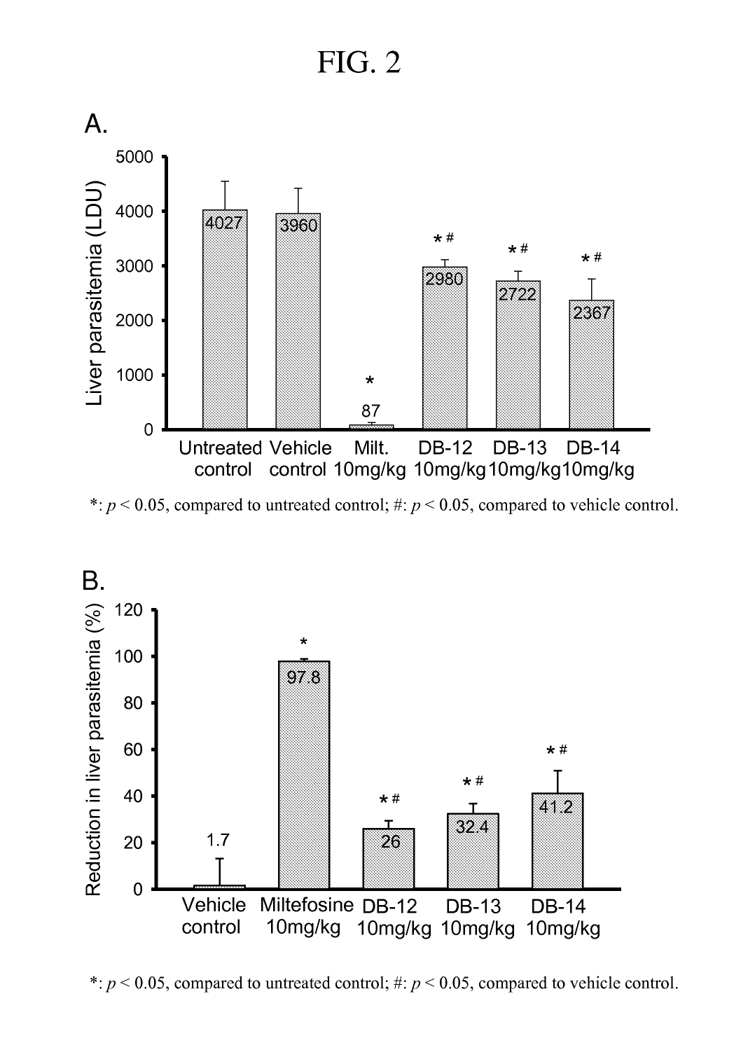

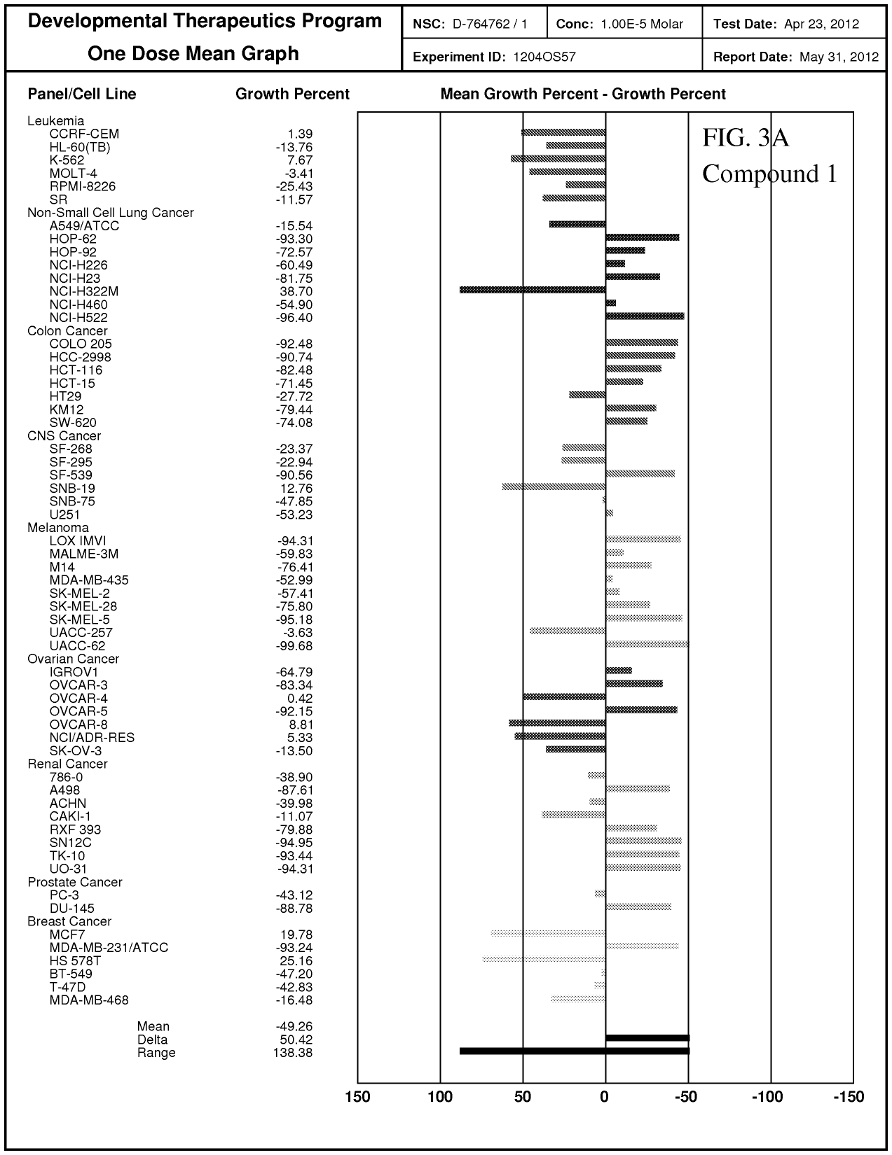

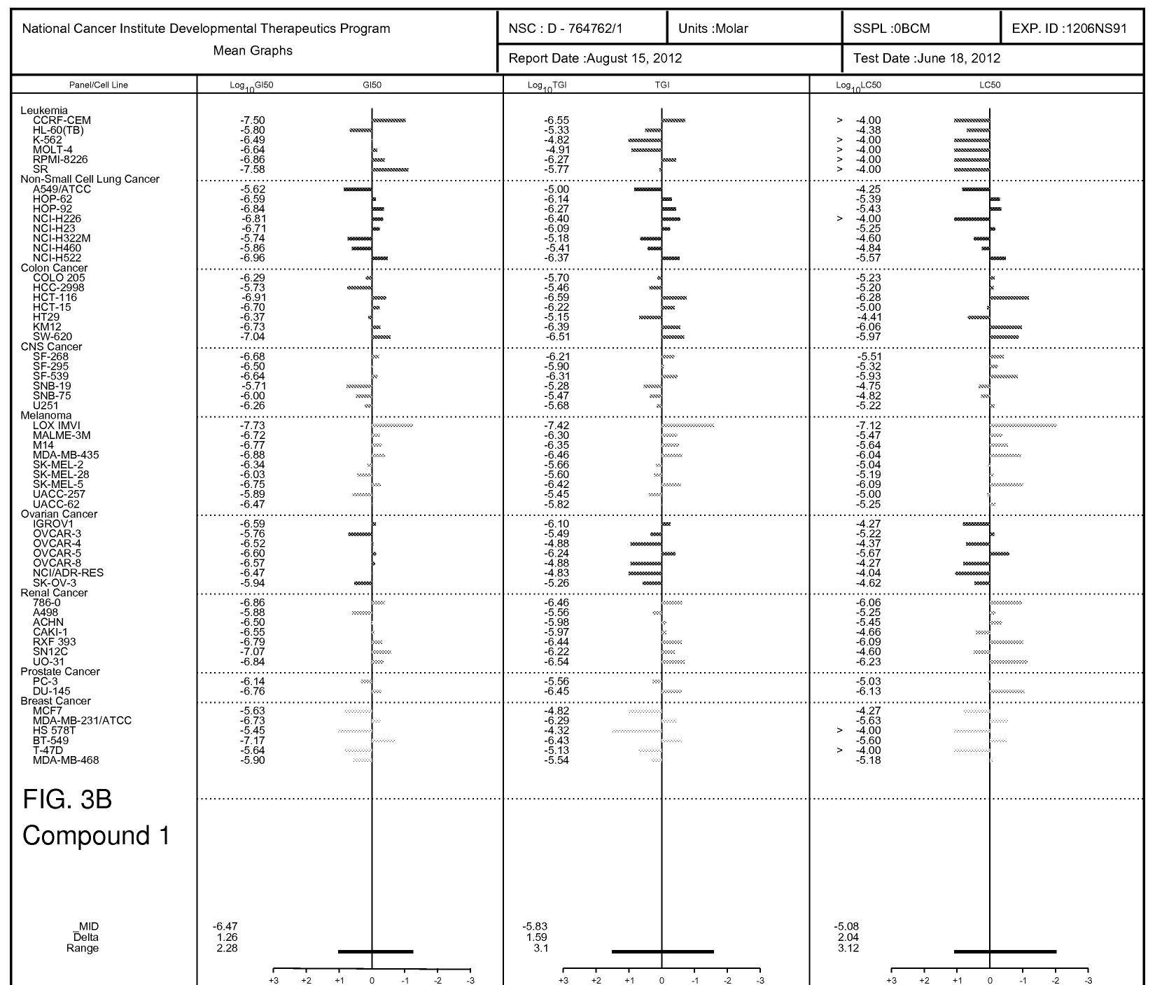

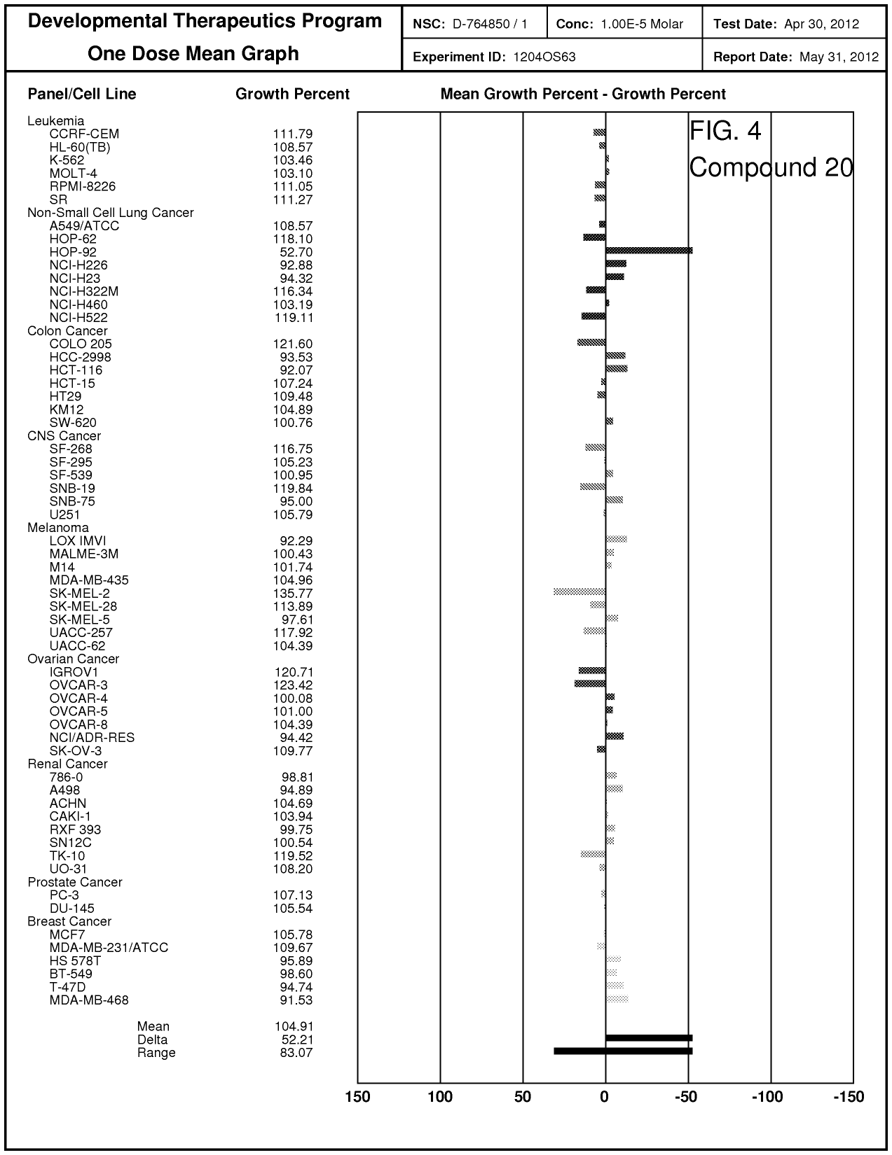

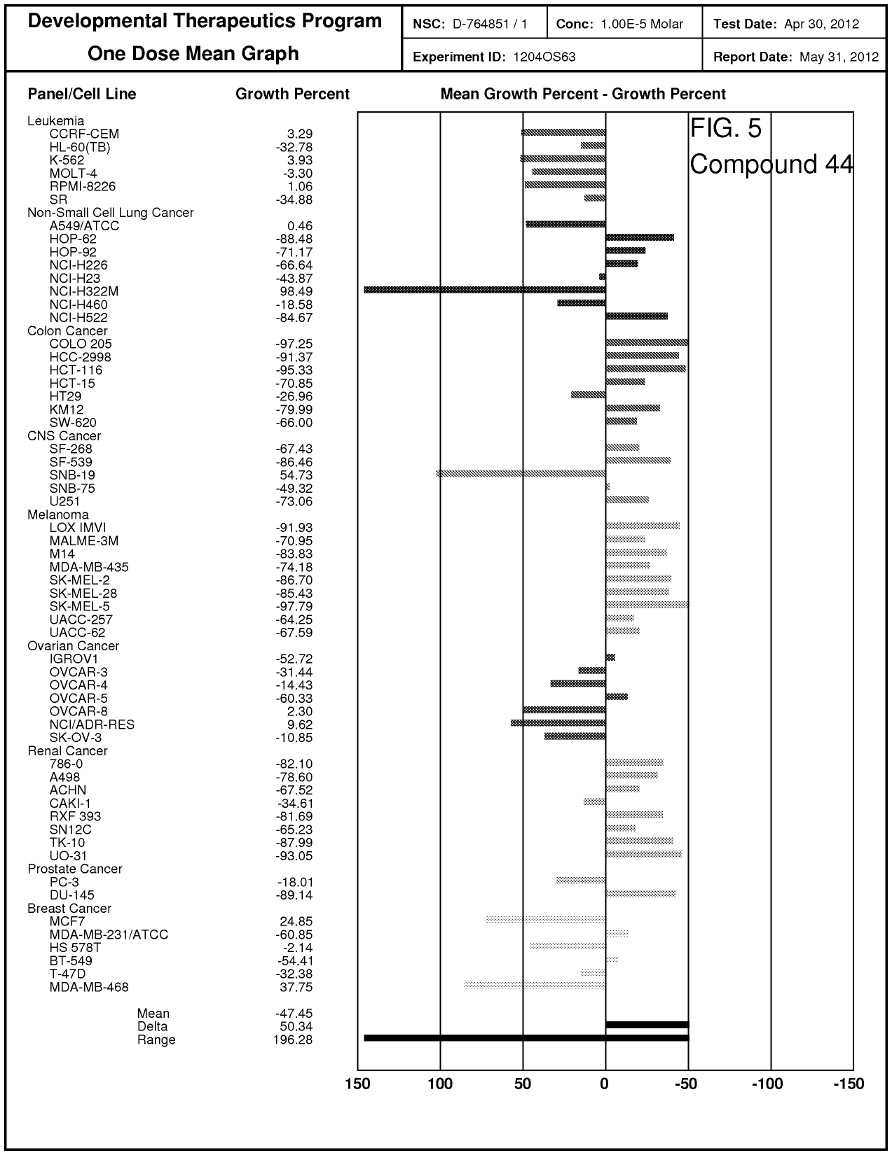

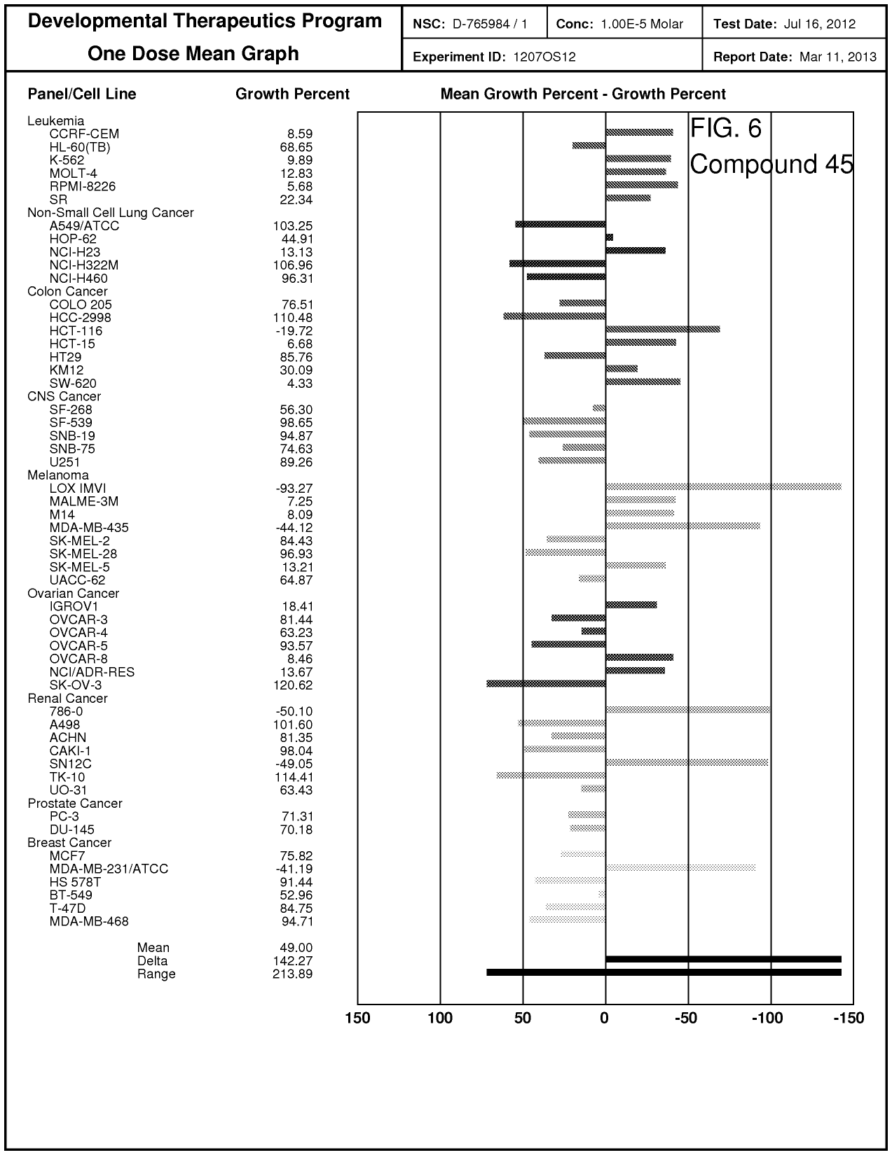

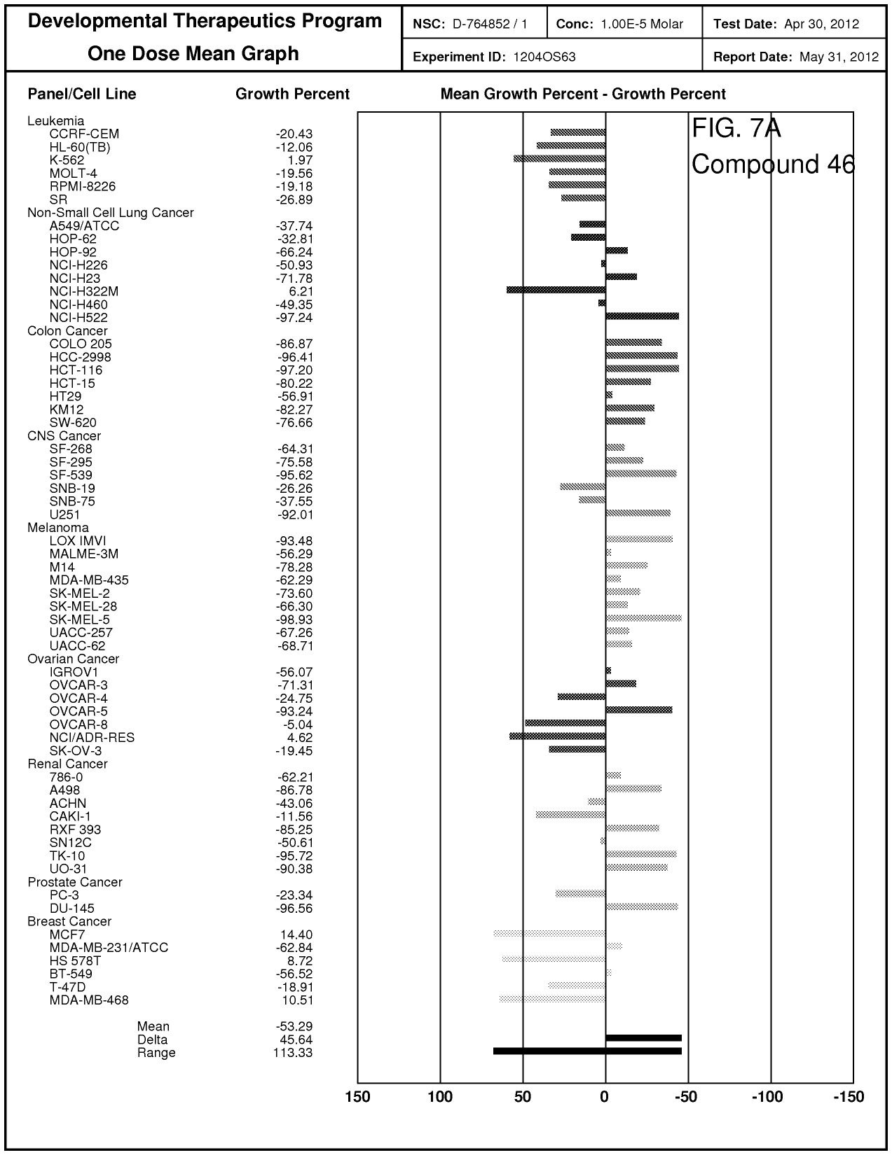

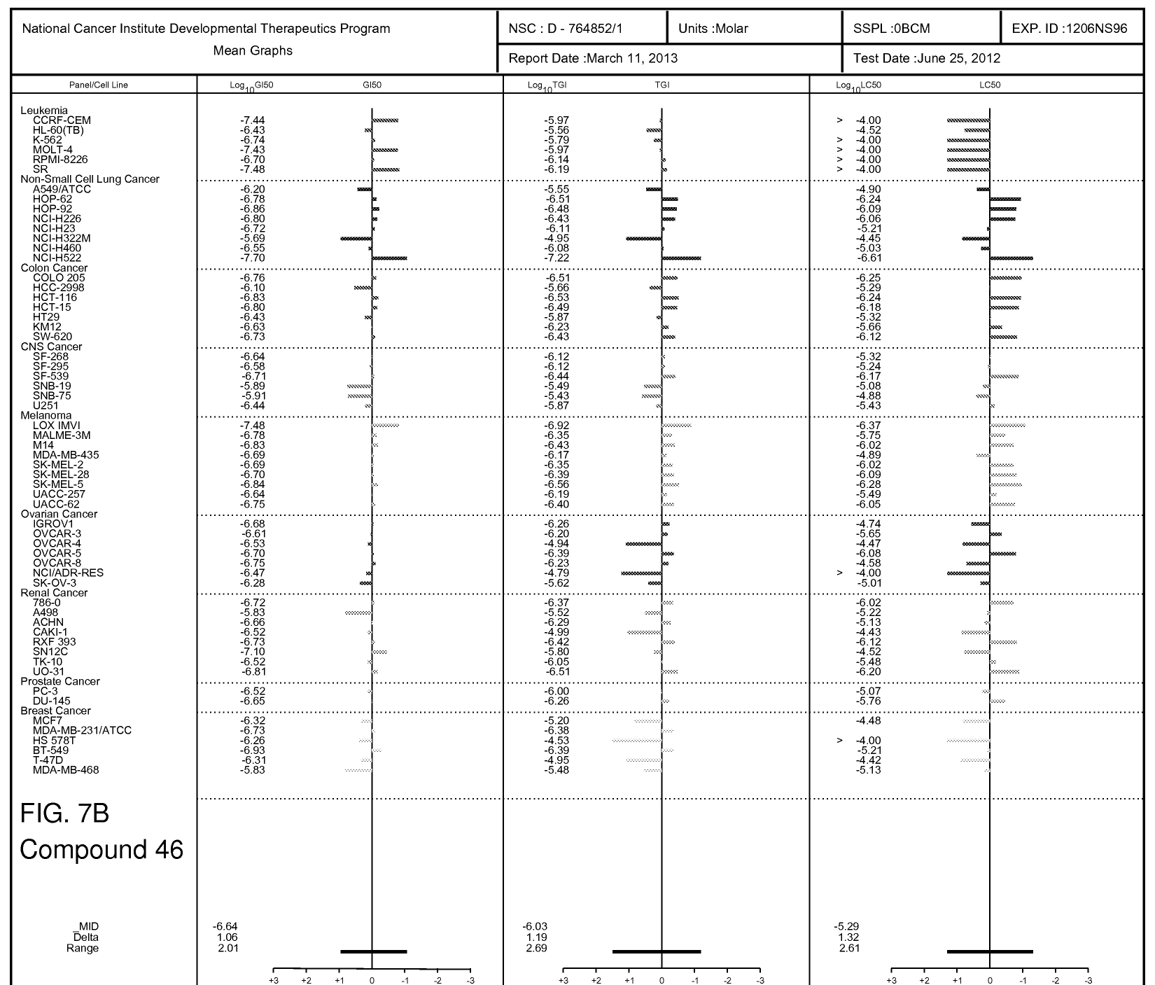

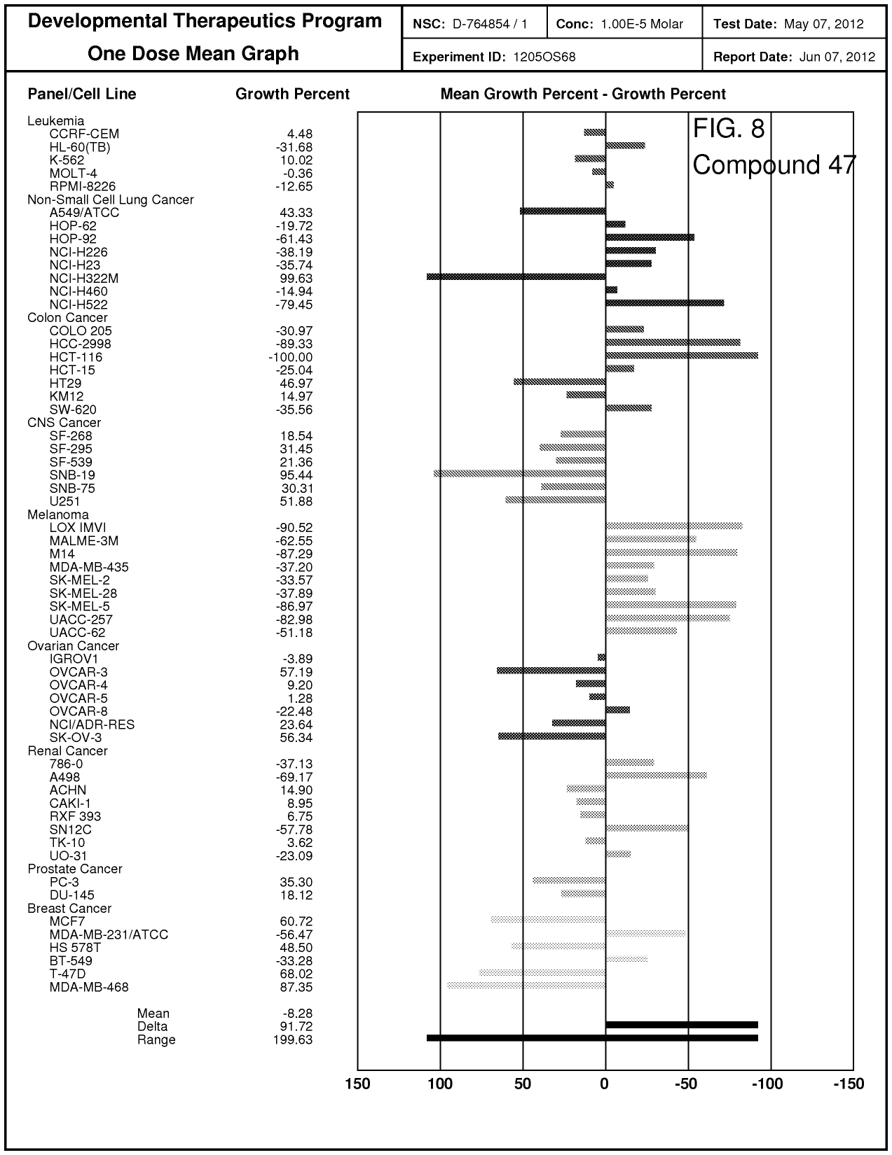

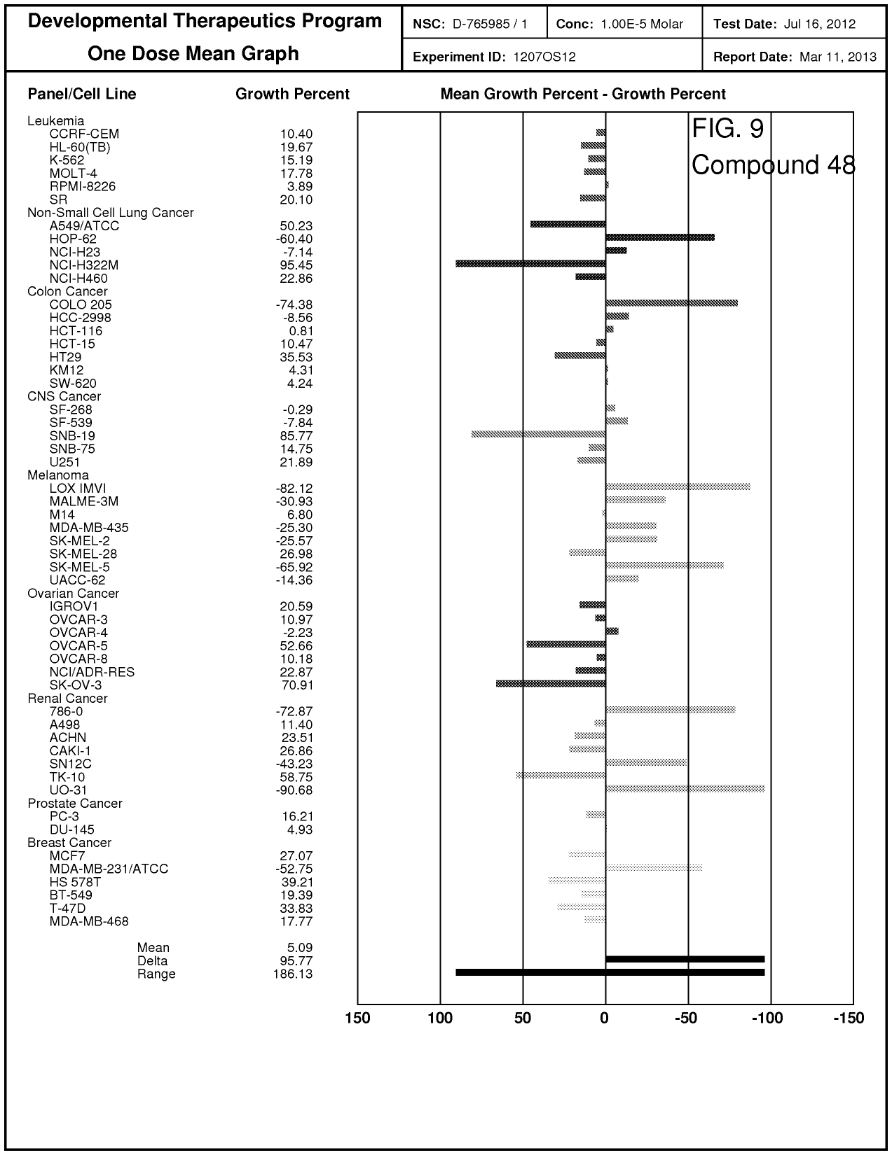

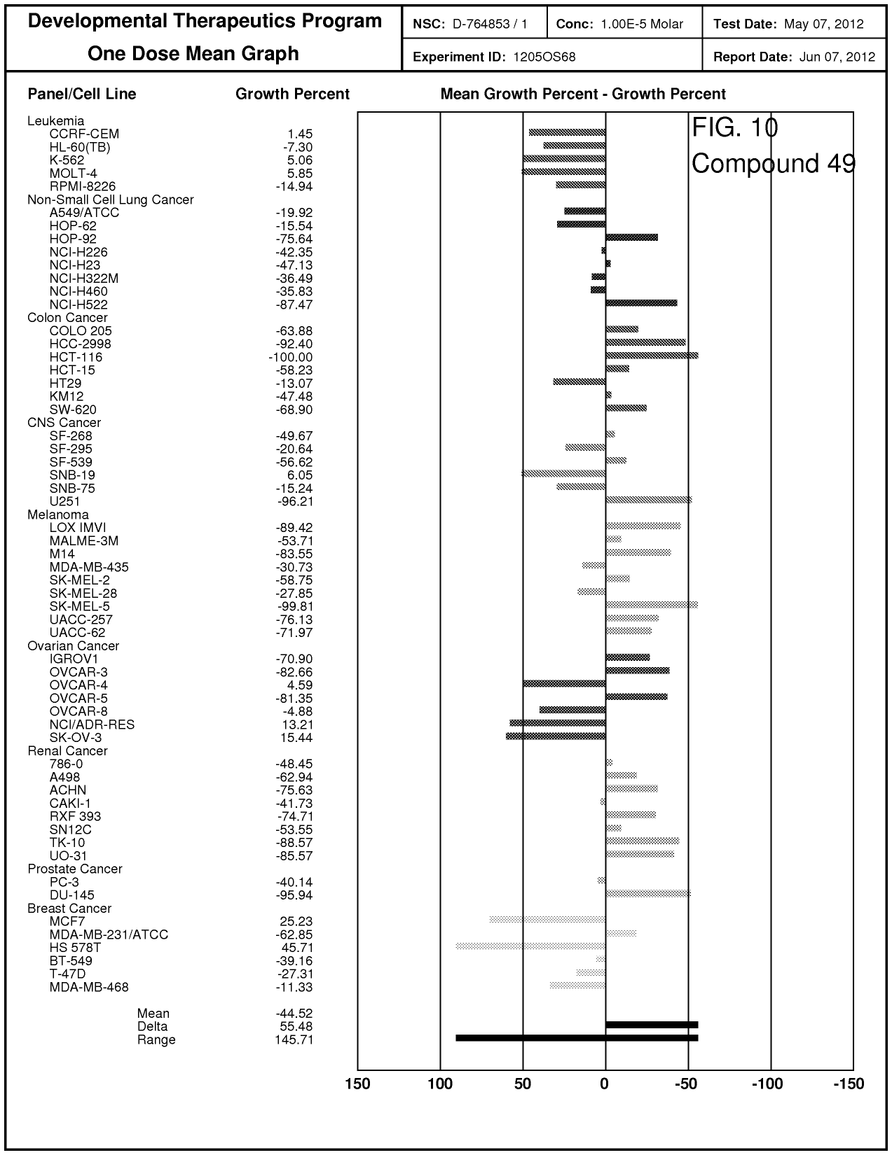

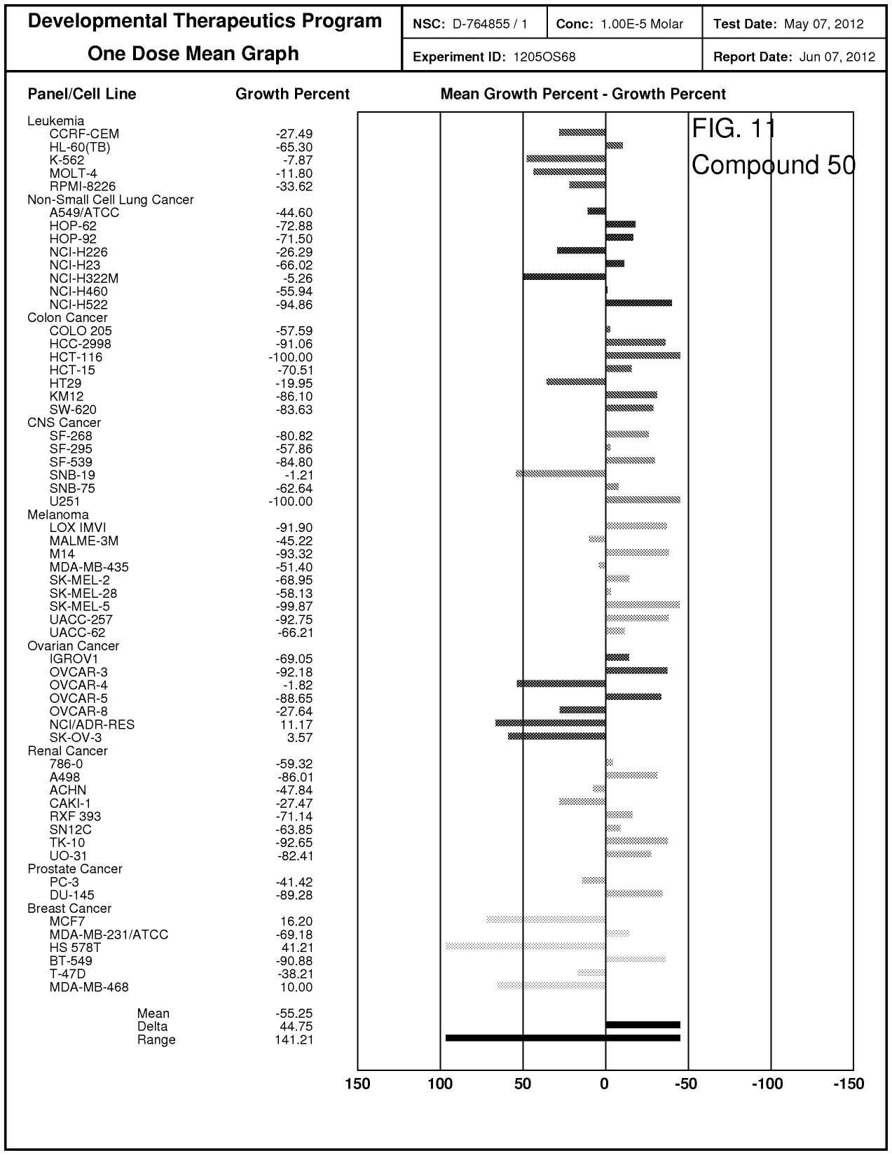

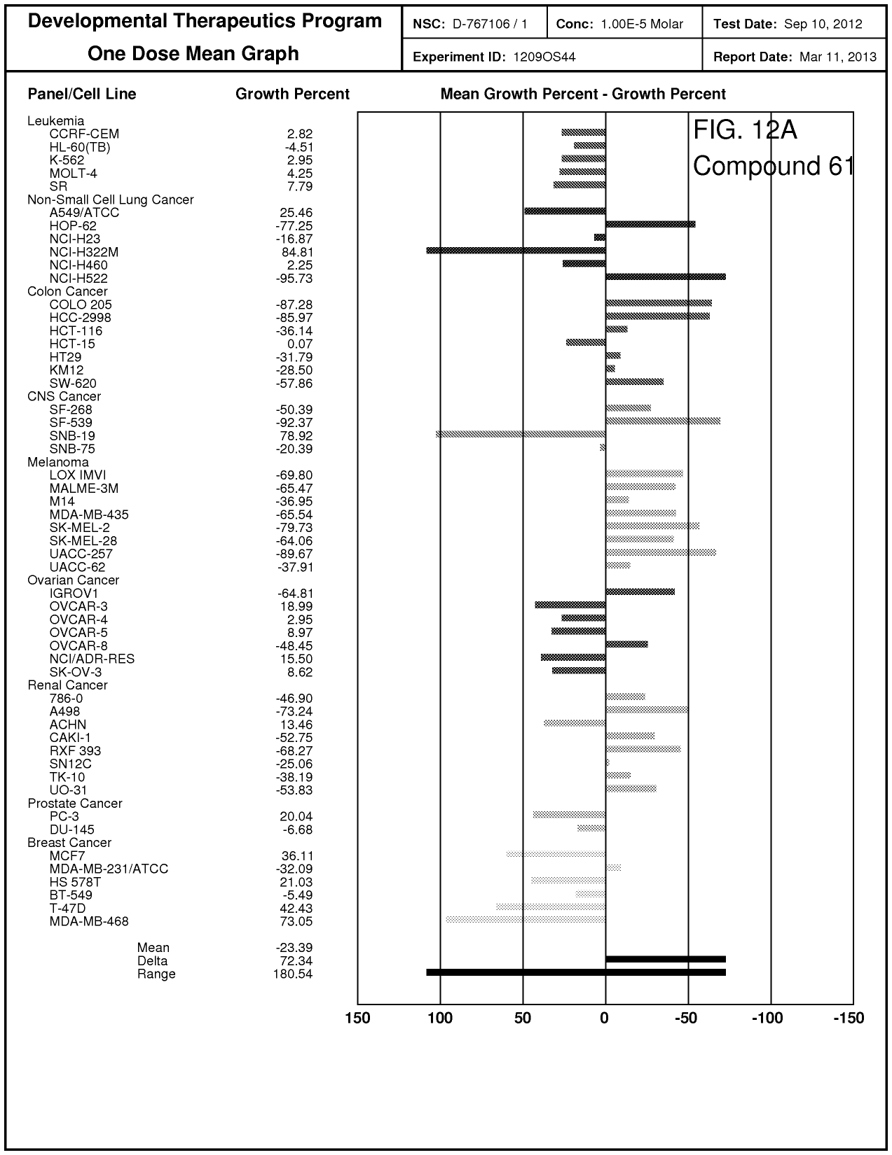

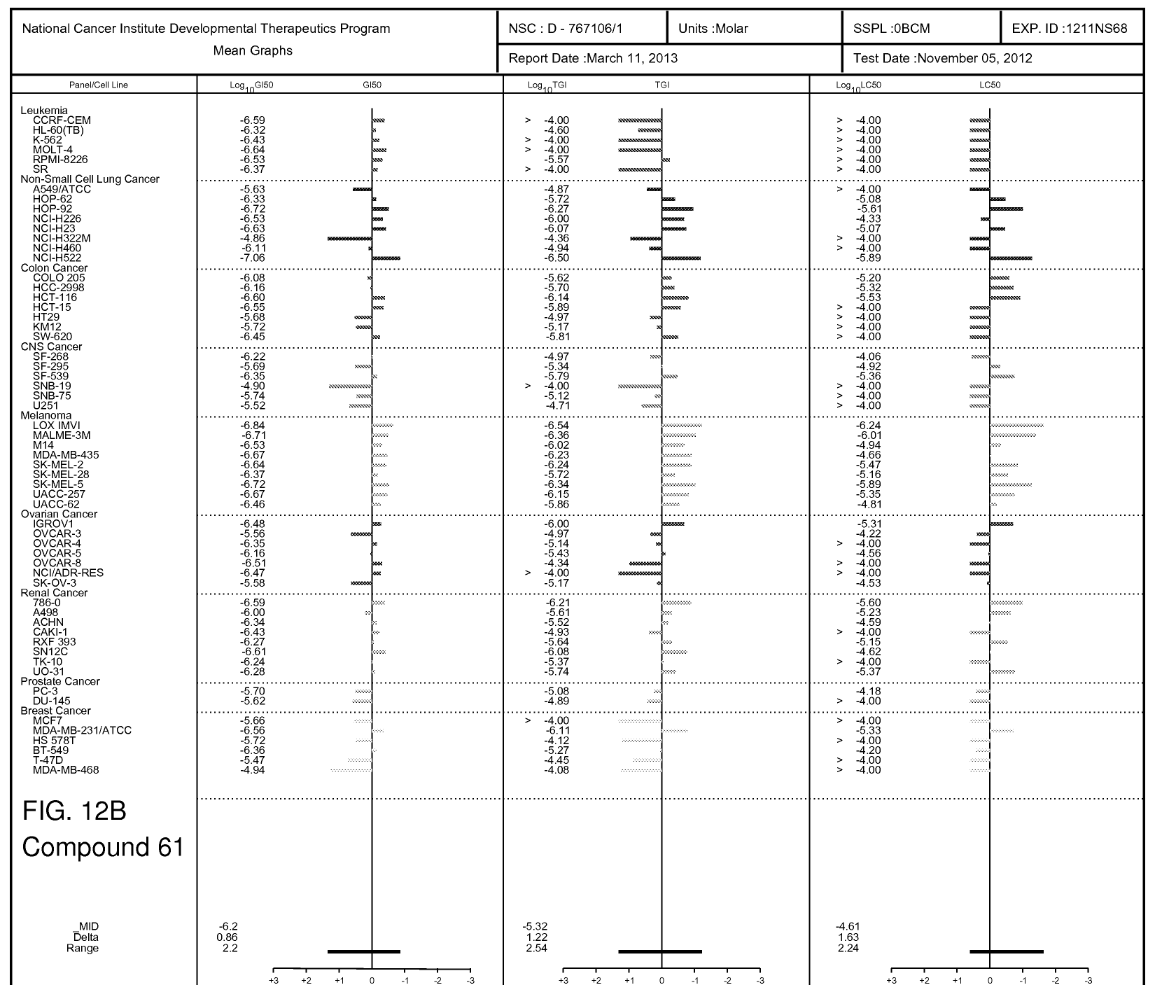

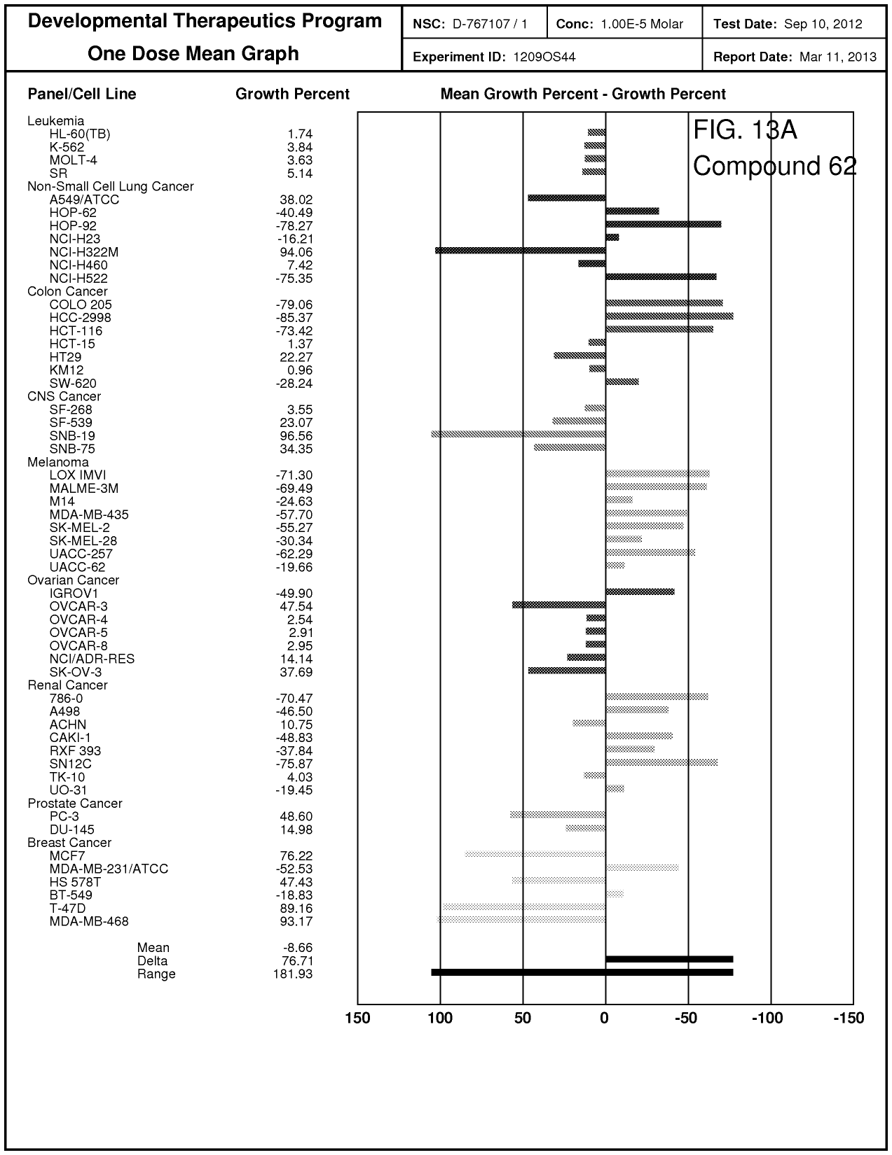

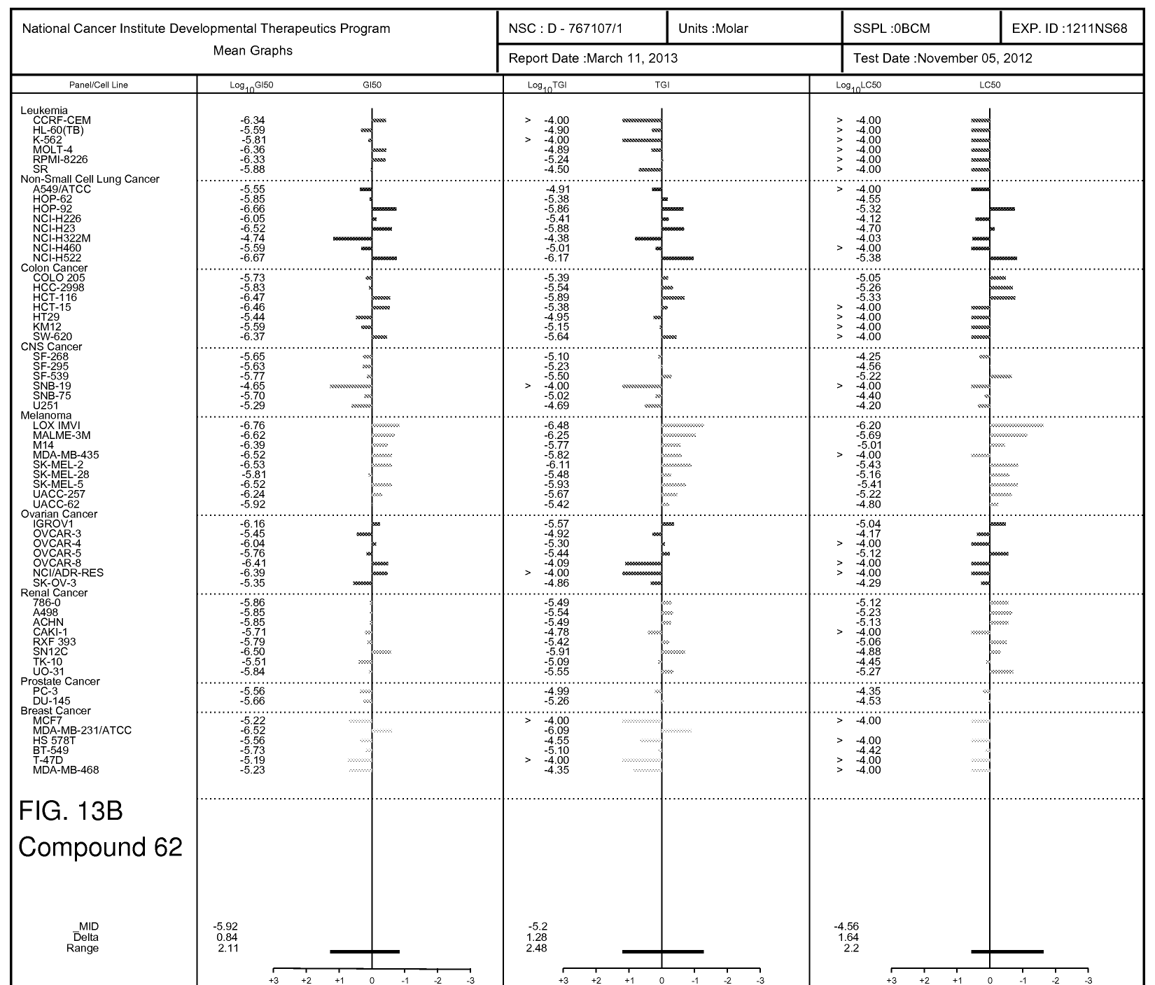

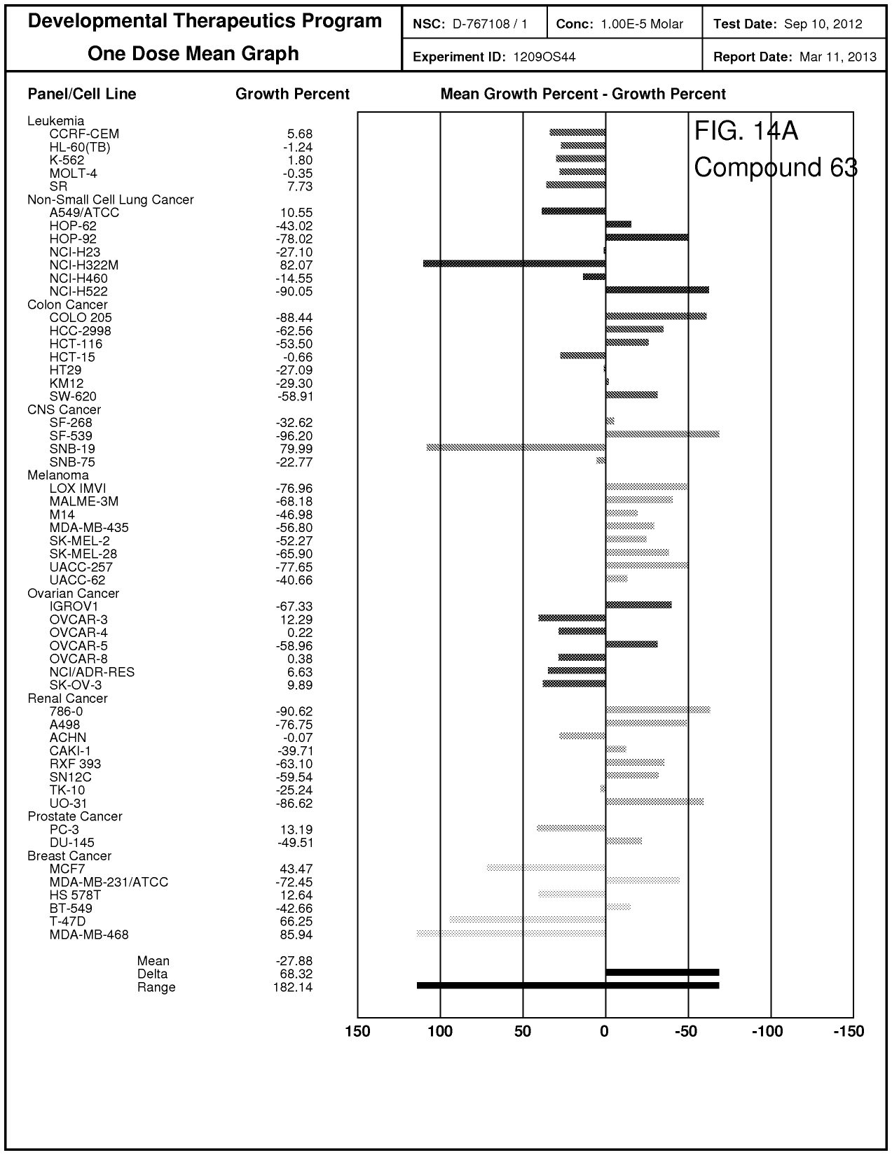

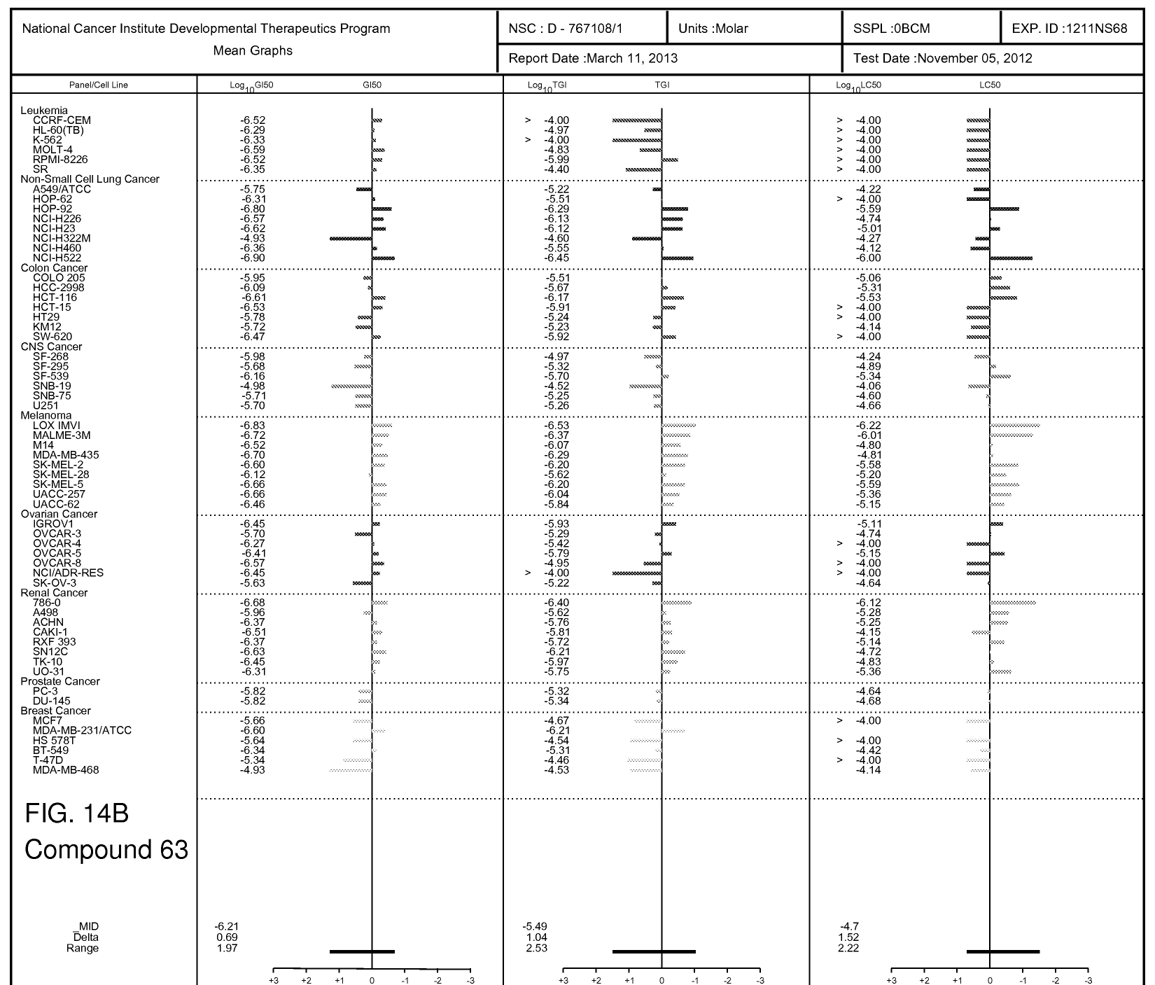

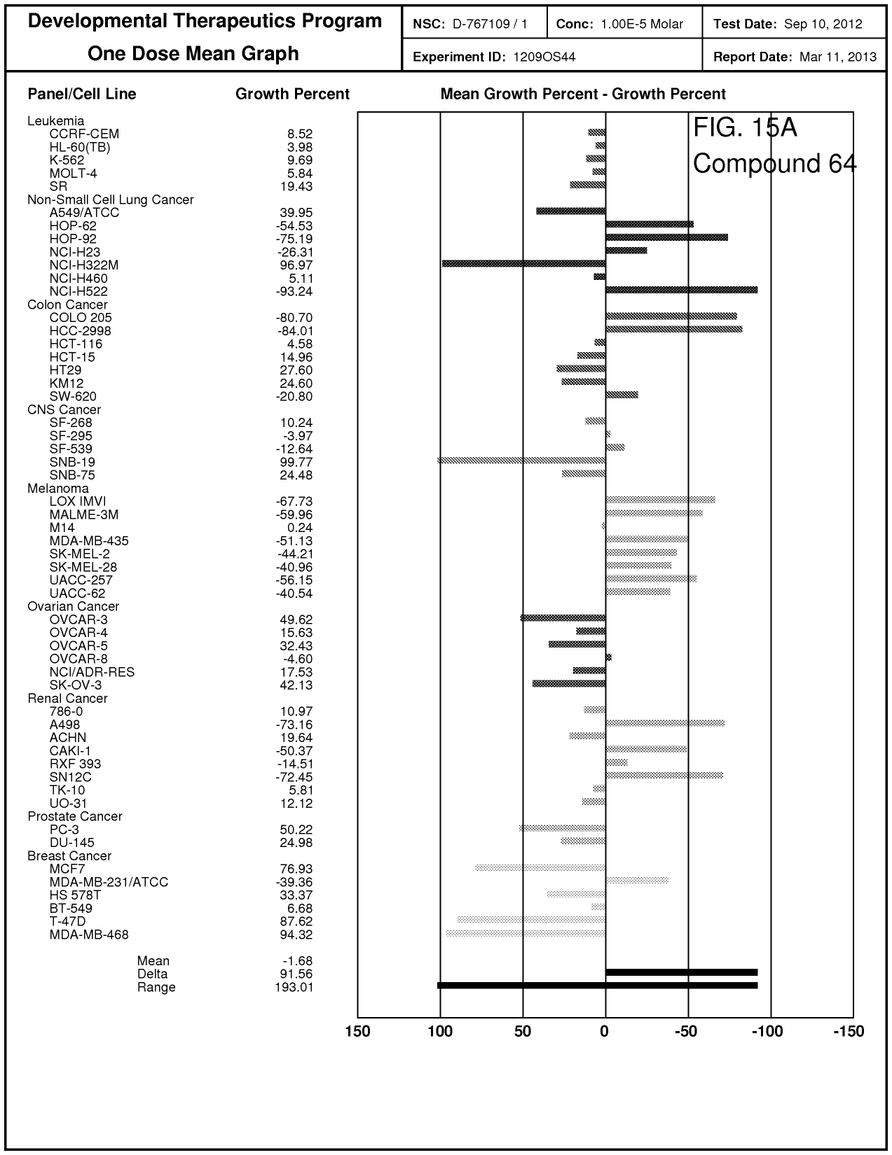

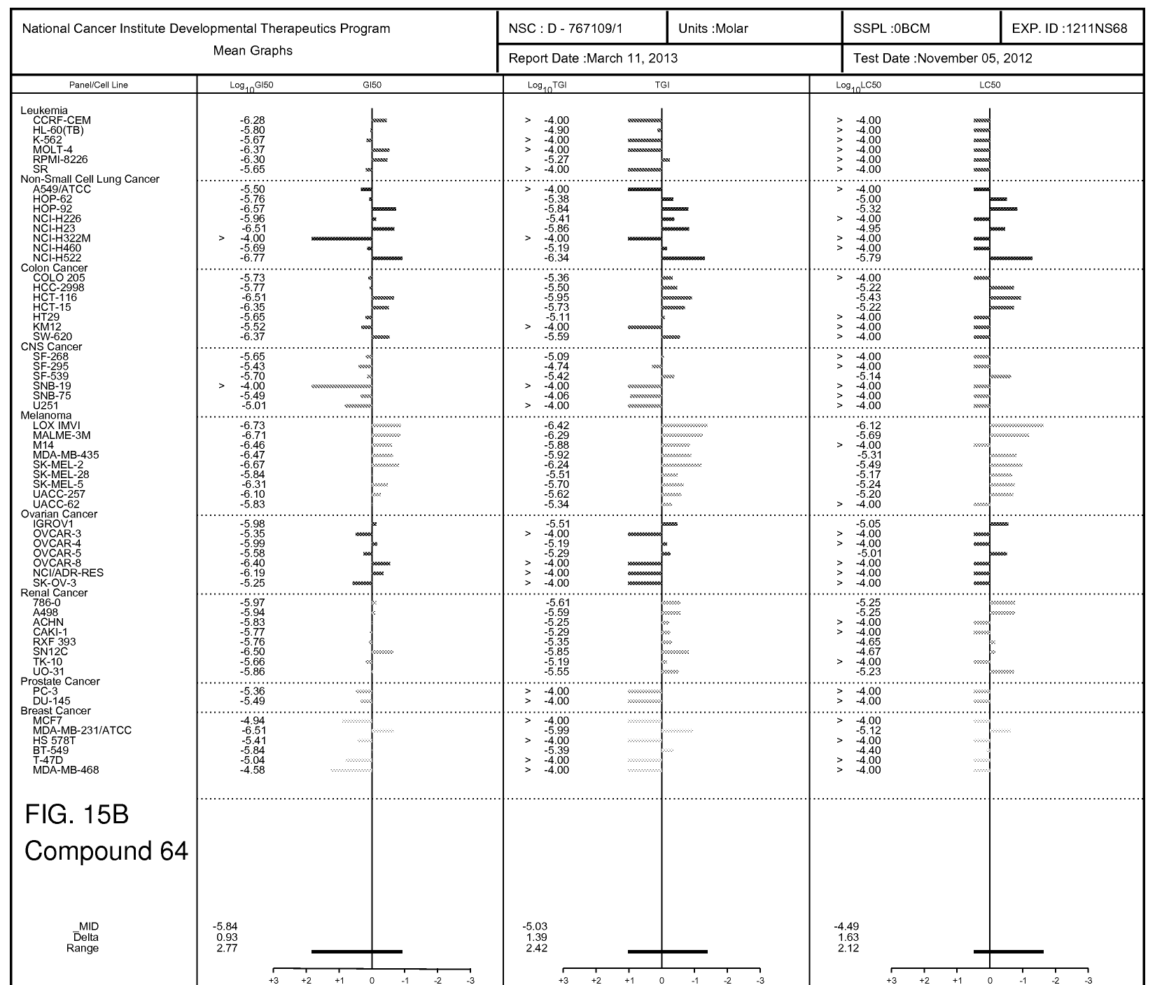

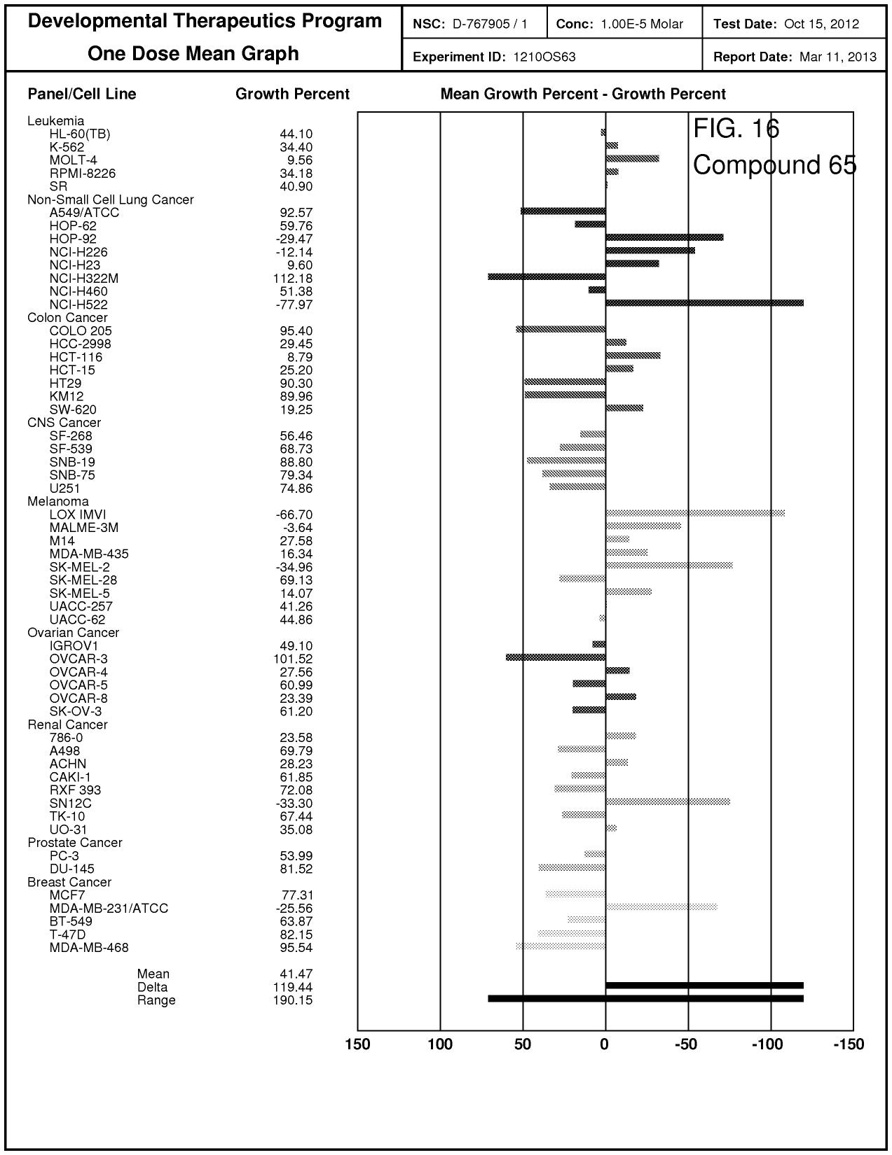

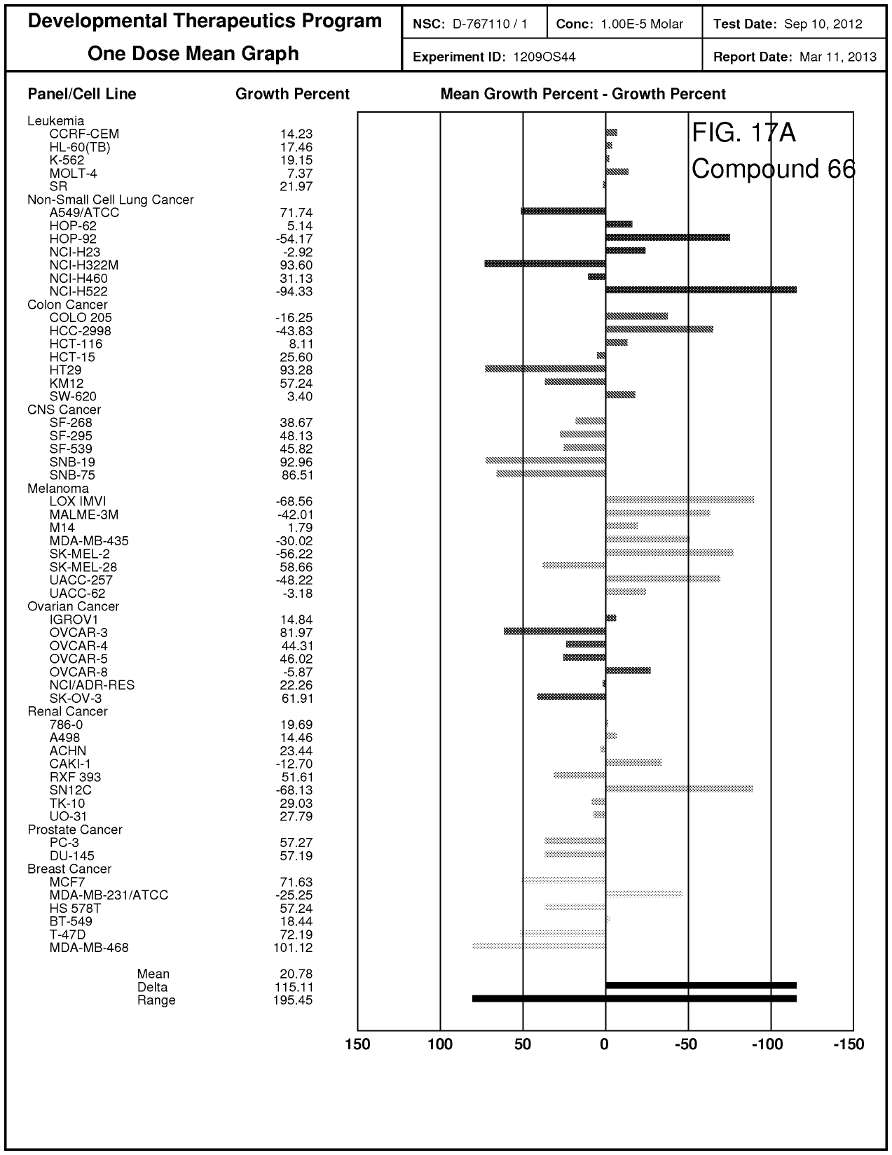

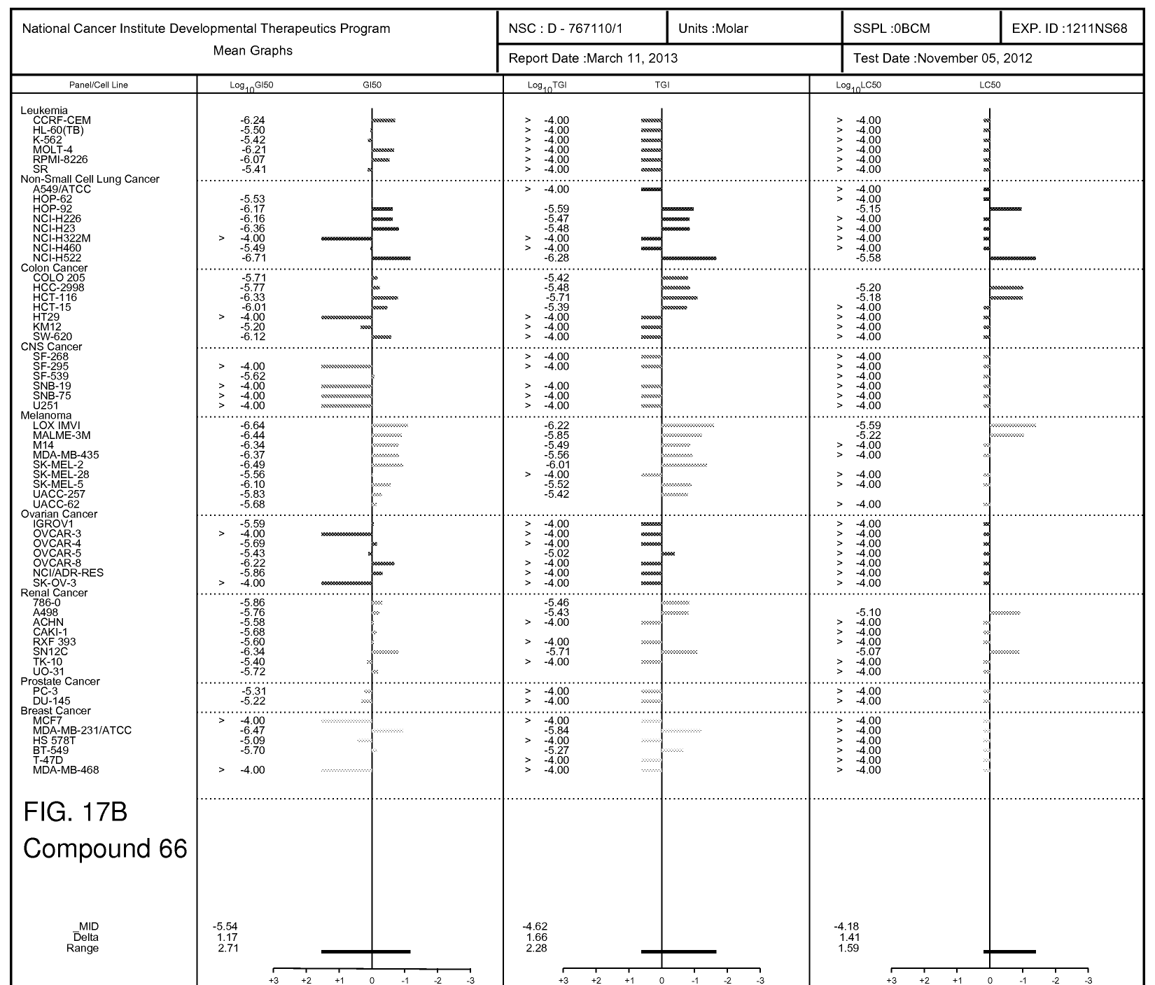

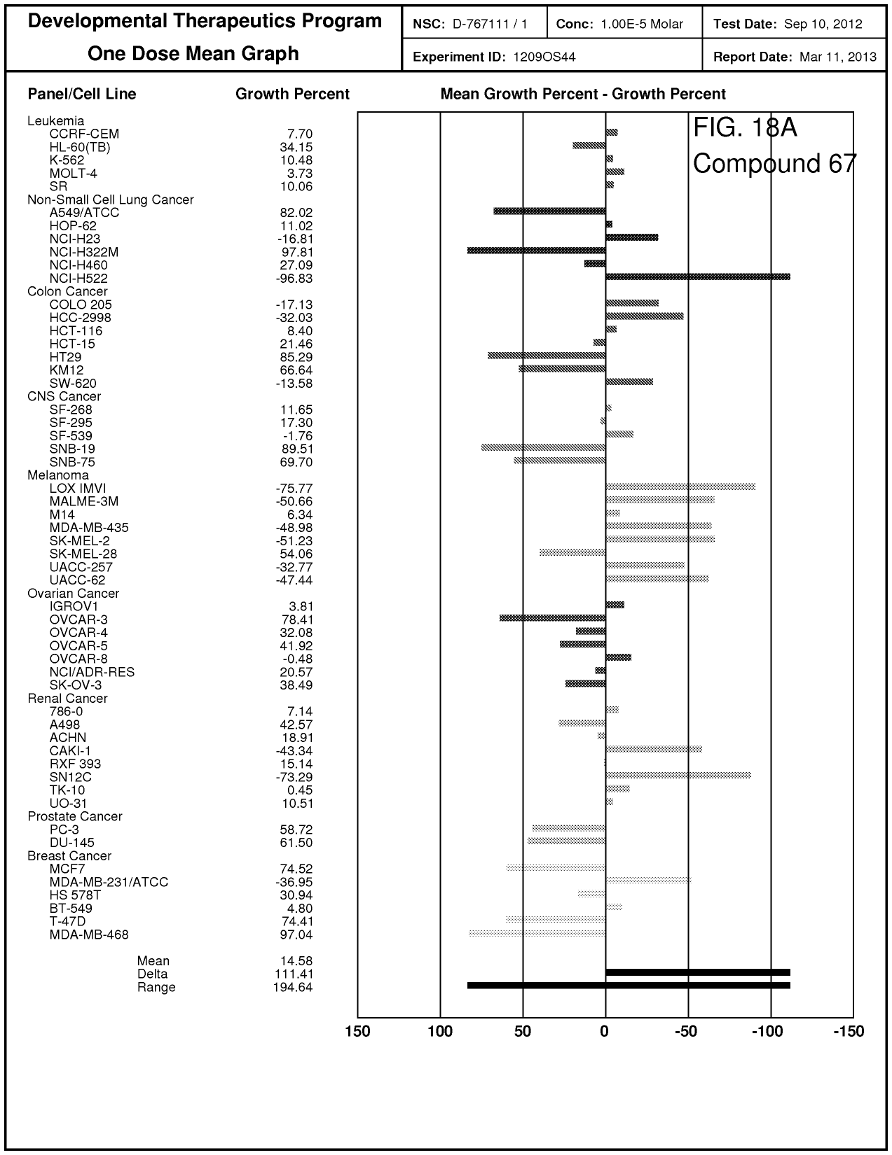

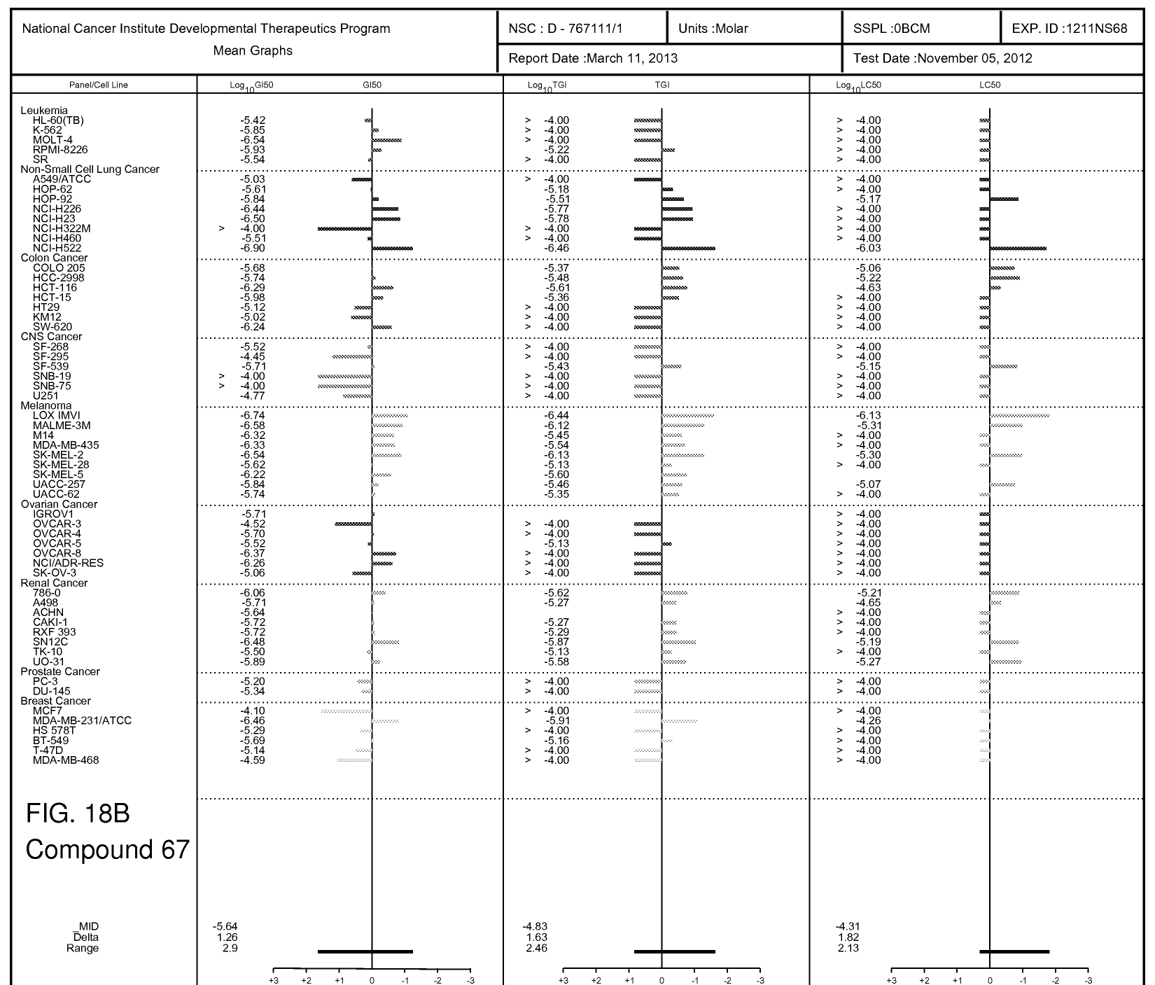

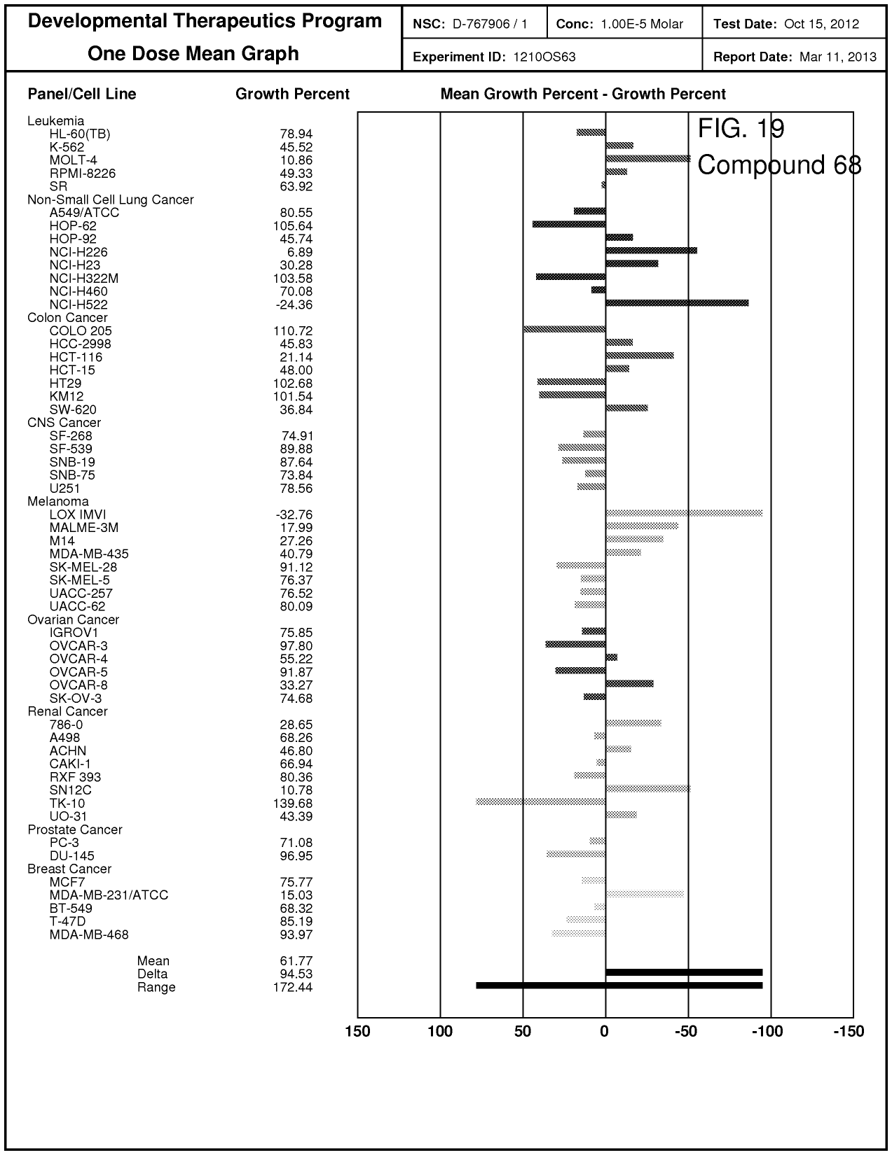

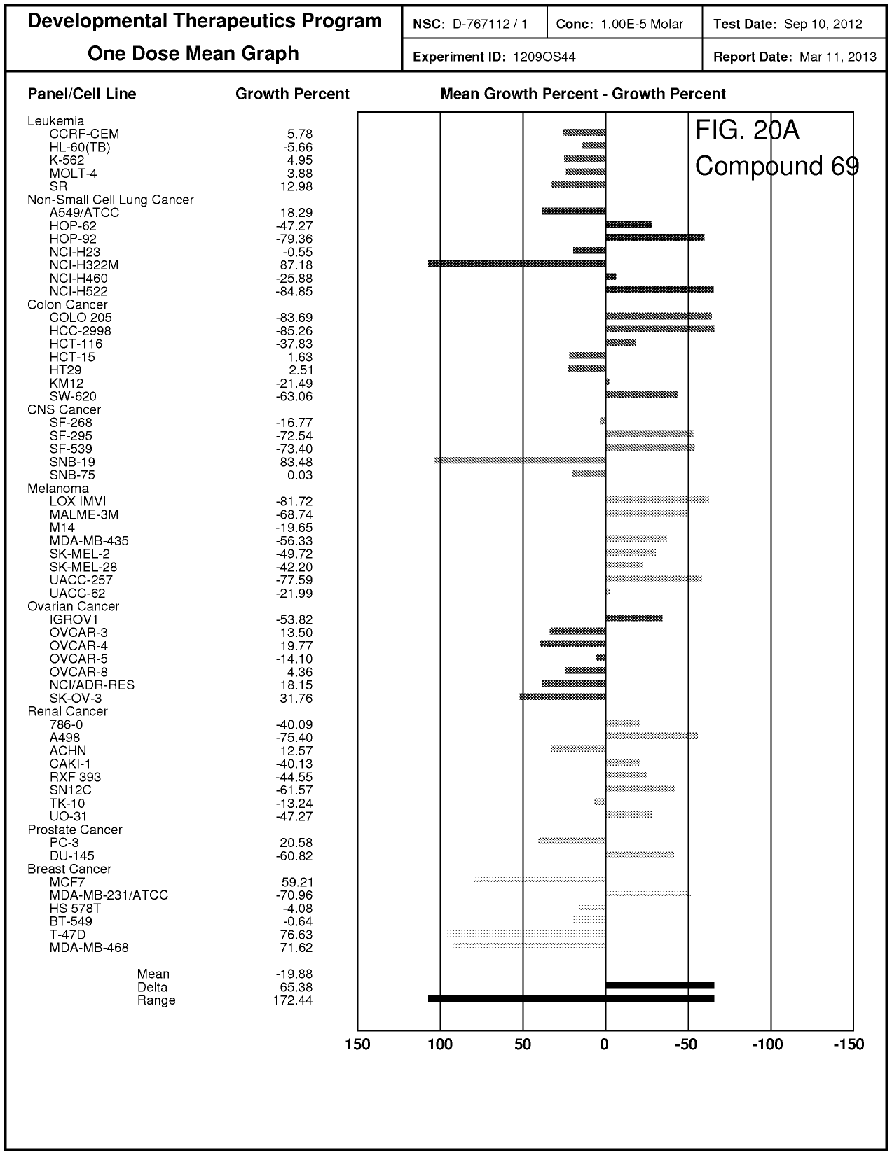

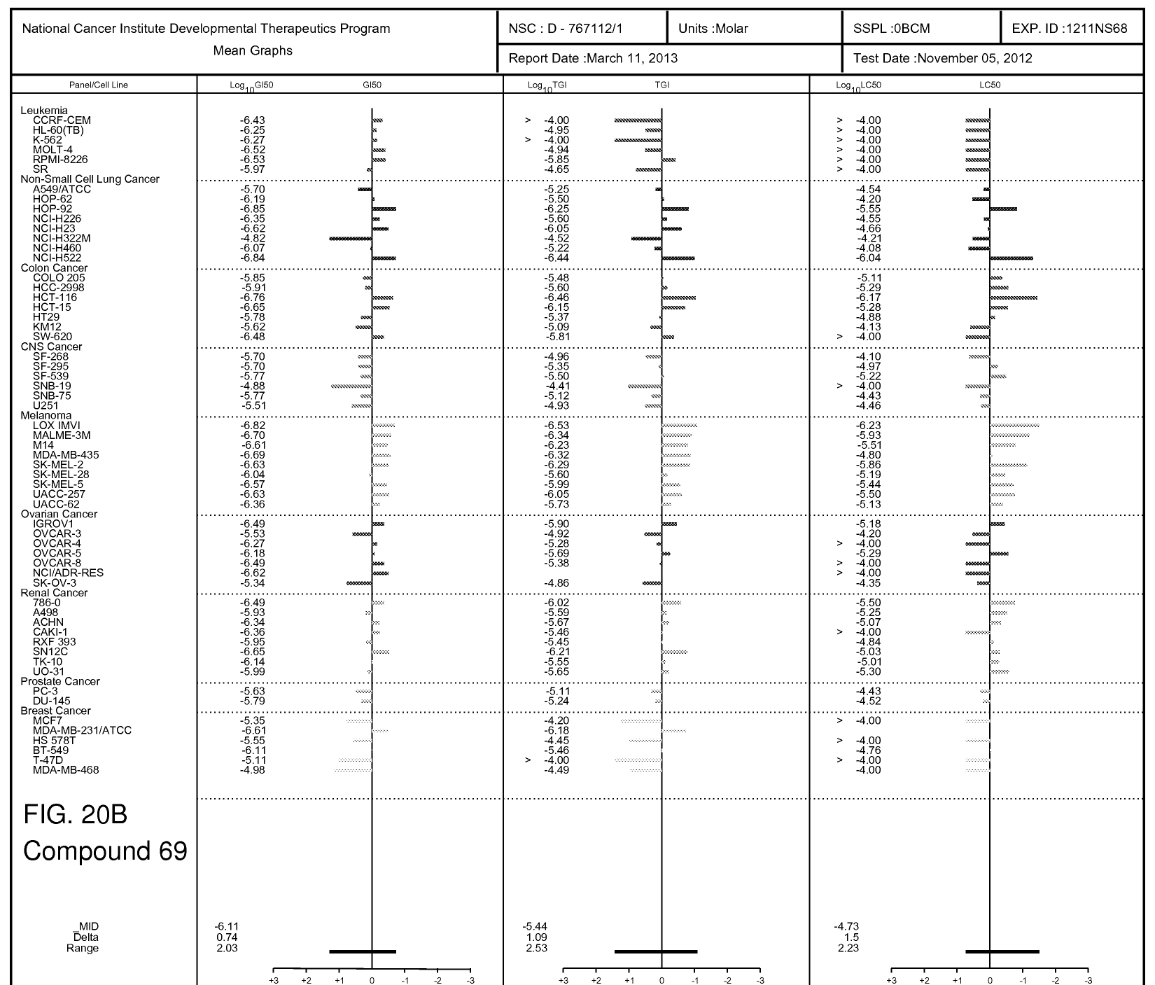

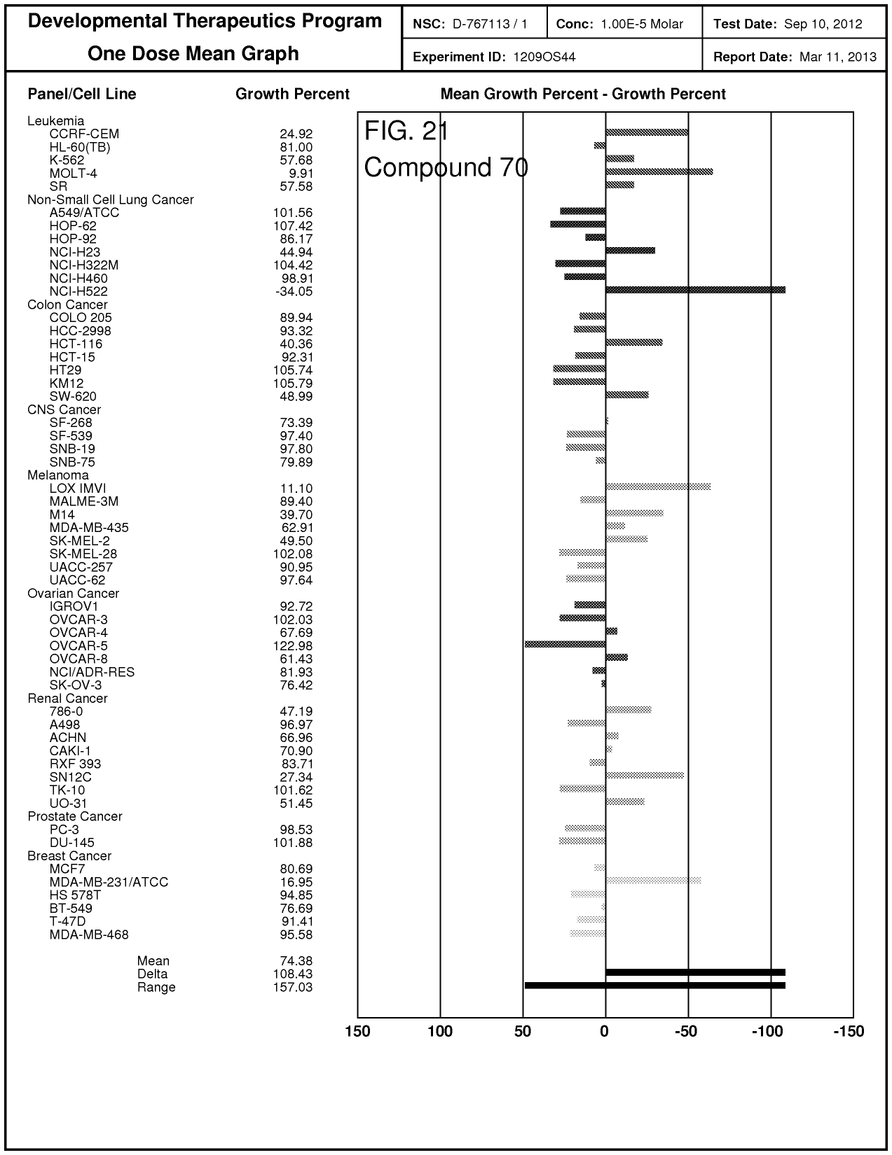

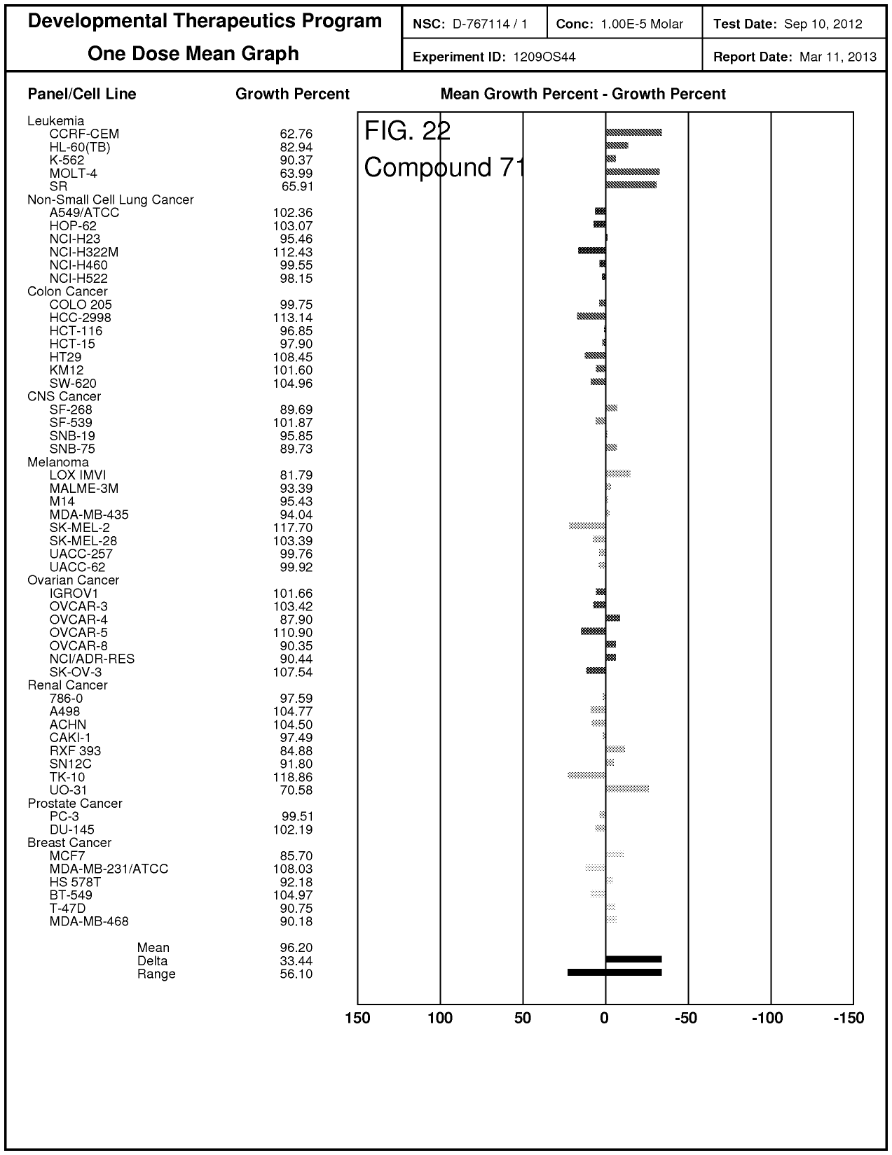

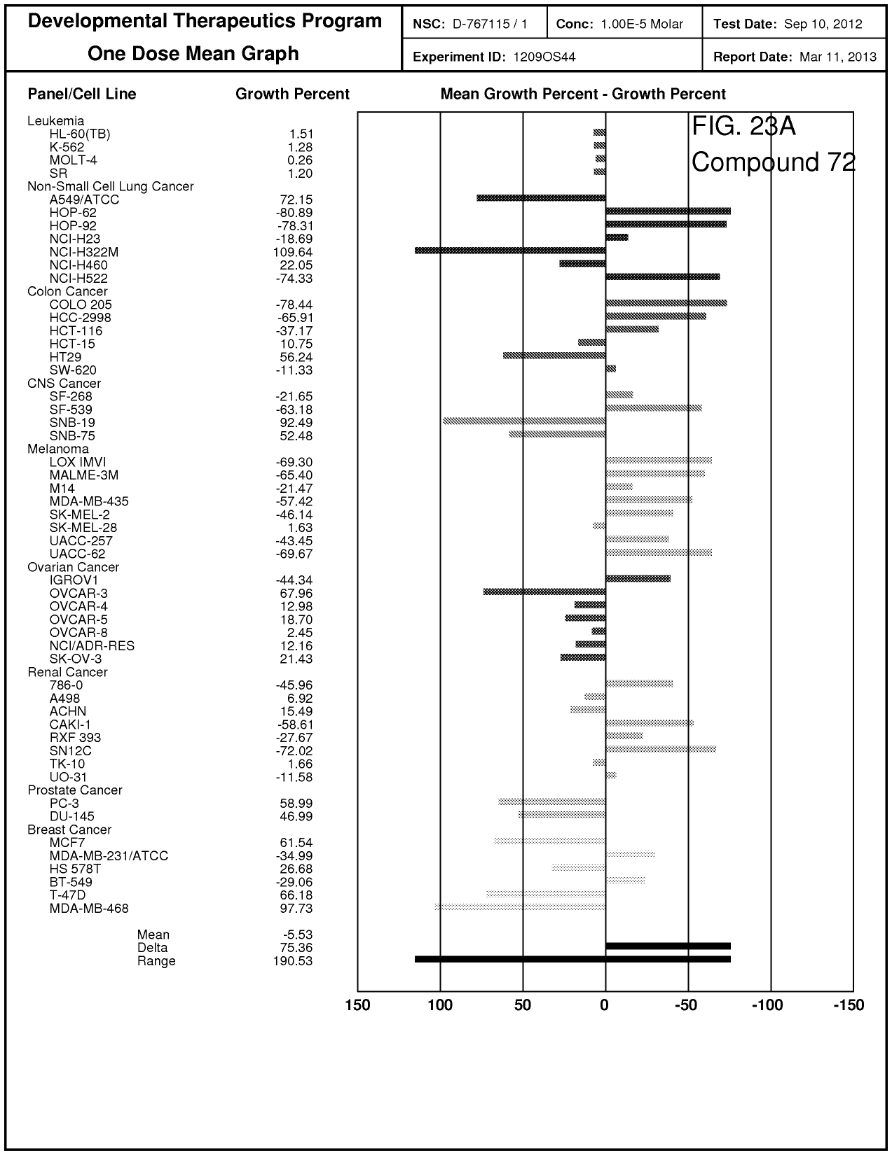

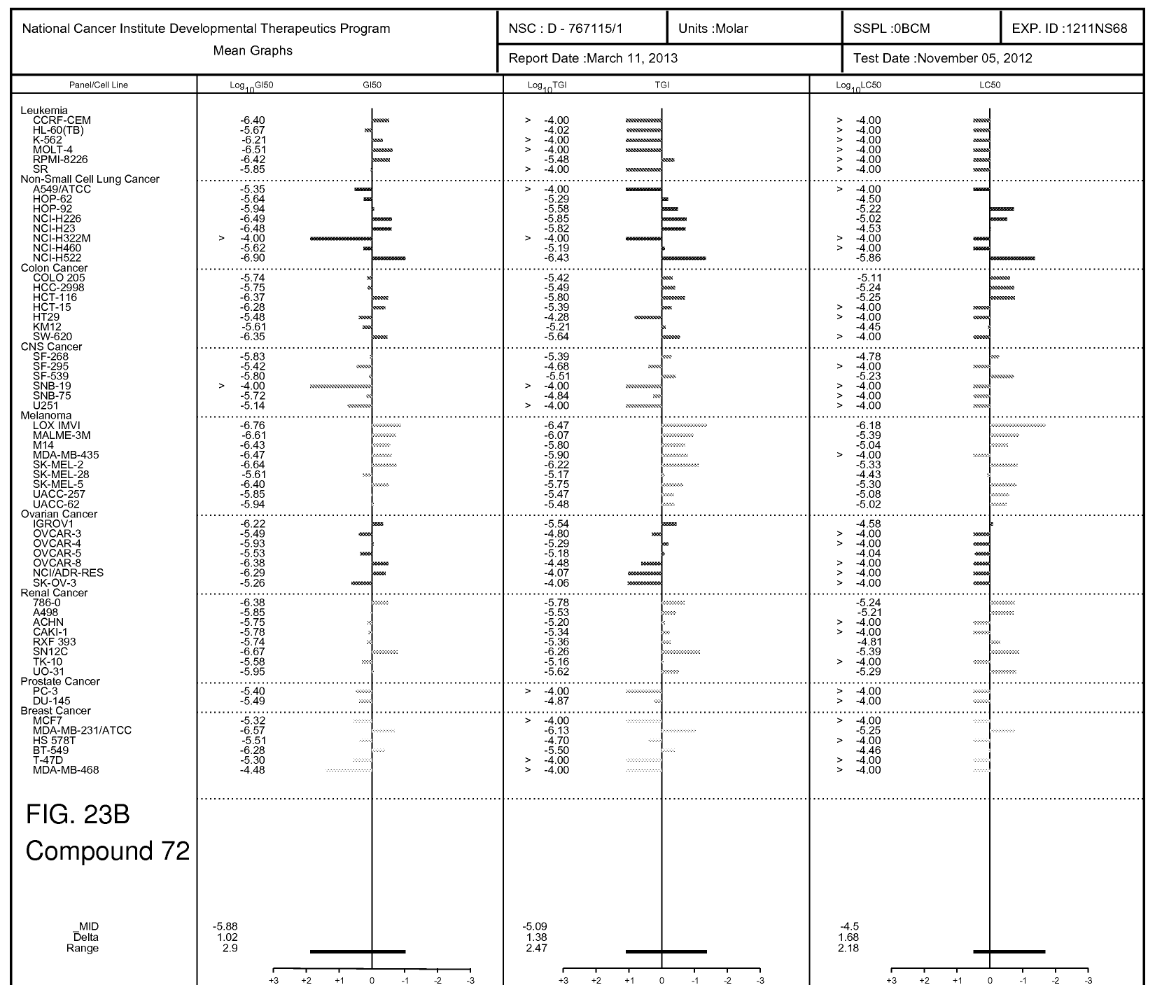

Compounds 47, 48, and 50 were then evaluated in animal studies with BALB/c mice with leishmaniasis. Compounds were delivered via i.p. injection at 10 mg/kg, with untreated, vehicle, and miltefosine controls as described in Table 2. As shown in Tables 3-6 and FIG. 2, compounds 47, 48, and 50 all significantly reduced liver parasitemia in the infected BALB/c mice with reduction percentages of 26%, 32%, and 41%, respectively. The vehicle control group showed a 2% reduction in liver parasitemia. Miltefosine, the only oral treatment currently available to treat leishmaniasis in humans, was also evaluated in the BALB/c mice for comparison purposes. Miltefosine showed a 98% reduction in liver parasitemia in the infected mice.

TABLE 3

| | | | In- | No. of | Mean | Dose |
|---|---|---|---|---|---|---|
| | | | fection | LV82 | Body | (Nov. 28, 2011 |
| Group | n | DOB | Date | PM | Weight(g) | to Dec. 2, 2011) |
| A | 4 | Sep. | Nov. | 5 × 10⁷/ | 17.2 | PBS |
| (Untreated | | 23, | 21, | mouse | 19.2 | 200 μL/mouse |
| Control) | | 2011 | 2011 | i.v. | 17.7 | i.p. |
| | | | | | 18.4 | |
| B | 4 | Sep. | Nov. | 5 × 10⁷/ | 18.2 | 90 μL |
| (Vehicle | | 23, | 21, | mouse | 17.4 | 90 μL |
| control) | | 2011 | 2011 | i.v. | 20.0 | 100 μL |
| | | | | | 19.7 | 100 μL |
| C | 4 | Sep. | Nov. | 5 × 10⁷/ | 18.1 | 1 mg/mL, 180 μL |
| (Miltefosine; | | 23, | 21, | mouse | 18.5 | 1 mg/mL, 190 μL |
| 10 mg/kg) | | 2011 | 2011 | i.v. | 17.2 | 1 mg/mL, 170 μL |
| | | | | | 19.9 | 1 mg/mL, 200 μL |
| D | 4 | Sep. | Nov. | 5 × 10⁷/ | 18.7 | 2 mg/mL, 90 μL |
| (Compound | | 23, | 21, | mouse | 20.4 | 2 mg/mL, 100 μL |
| 47; | | 2011 | 2011 | i.v. | 18.9 | 2 mg/mL, 90 μL |
| 10 mg/kg) | | | | | 18.8 | 2 mg/mL, 90 μL |
| E | 4 | Sep. | Nov. | 5 × 10⁷/ | 17.3 | 2 mg/mL, 90 μL |
| (Compound | | 23, | 21, | mouse | 20.0 | 2 mg/mL, 100 μL |
| 48; | | 2011 | 2011 | i.v. | 20.1 | 2 mg/mL, 100 μL |
| 10 mg/kg) | | | | | 19.7 | 2 mg/mL, 100 μL |
| F | 4 | Sep. | Nov. | 5 × 10⁷/ | 18.1 | 2 mg/mL, 90 μL |
| (Compound | | 23, | 21, | mouse | 15.8 | 2 mg/mL, 80 μL |
| 50; | | 2011 | 2011 | i.v. | 15.6 | 2 mg/mL, 80 μL |
| 10 mg/kg) | | | | | 15.7 | 2 mg/mL, 80 μL |

TABLE 4

| | | | | | # of | | |
|---|---|---|---|---|---|---|---|
| | | Body weight | Liver Weight | Spleen weight | amastigotes | | LDU |
| Group | Mice | (g) | (g) | (g) | per 200 nuclei | LDU | mean ± SD |
| A | 1 | 17.6 | 0.8988 | 0.1366 | 742 | 3334.5 | 4026.7 ± |
| (Untreated | 2 | 19.7 | 1.1502 | 0.1416 | 760 | 4370.8 | 524.8 |
| Control) | 3 | 18.0 | 0.9989 | 0.1338 | 783 | 3910.7 | |
| | 4 | 19.0 | 1.1896 | 0.1492 | 755 | 4490.7 | |
| B | 1 | 18.0 | 0.9565 | 0.1268 | 771 | 3687.3 | 3959.9 ± |
| (Vehicle | 2 | 17.6 | 0.9910 | 0.1173 | 699 | 3463.5 | 461.6 |
| control) | 3 | 19.1 | 1.0647 | 0.1433 | 796 | 4237.5 | |
| | 4 | 20.0 | 1.1998 | 0.1542 | 742 | 4451.3 | |
| C | 1 | 18.3 | 0.9968 | 0.1400 | 22 | 109.6 | 87.0 ± |
| (miltefosine; | 2 | 18.0 | 1.0414 | 0.1394 | 13 | 67.7 | 43.9 |
| 10 | 3 | 17.1 | 0.8965 | 0.1324 | 8 | 35.9 | |
| mg/kg) | 4 | 20.0 | 1.1241 | 0.1606 | 24 | 134.9 | |
| D | 1 | 18.3 | 0.9934 | 0.1244 | 596 | 2960.3 | 2979.6 ± |
| (Compound | 2 | 19.5 | 1.0441 | 0.1357 | 543 | 2834.7 | 135.5 |
| 47; 10 | 3 | 18.3 | 0.9953 | 0.1074 | 595 | 2961.0 | |
| mg/kg) | 4 | 18.2 | 1.0867 | 0.1255 | 582 | 3162.3 | |
| E | 1 | 17.2 | 0.9483 | 0.1187 | 536 | 2541.4 | 2721.9 ± |
| (Compound | 2 | 19.2 | 1.0700 | 0.1399 | 529 | 2830.2 | 177.9 |
| 48; 10 | 3 | 19.8 | 1.1699 | 0.1532 | 498 | 2913.1 | |
| mg/kg) | 4 | 19.0 | 1.0937 | 0.1400 | 476 | 2603.0 | |
| F | 1 | 15.7 | 0.8718 | 0.1048 | 628 | 2737.5 | 2367.2 ± |
| (Compound | 2 | 14.6 | 0.7698 | 0.1025 | 557 | 2143.9 | 391 |
| 50; 10 | 3 | 14.1 | 0.8258 | 0.1086 | 643 | 2654.9 | |
| mg/kg) | 4 | 13.4 | 0.6316 | 0.0673 | 612 | 1932.7 | |

TABLE 5

| | Mean body weight | Mean body weight |
|---|---|---|
| | of mice | of mice |
| Group | Pre-treatment (g) | Post-treatment (g) |
| A (Untreated control) | 18.1 | 18.6 |
| B (Vehicle control) | 18.8 | 18.7 |
| C (miltefosine; 10 mg/kg) | 18.4 | 18.4 |
| D (Compound 47; 10 mg/kg) | 19.2 | 18.6 |
| E (Compound 48; 10 mg/kg) | 19.3 | 18.8 |
| F (Compound 50; 10 mg/kg) | 16.3 | 14.5 |

TABLE 6

| Group | % Reduction (Average) | SD |
|---|---|---|
| B (Vehicle control) | 1.7 | 11.5 |
| C (Miltefosine; 10 mg/kg) | 97.8 | 1.1 |
| D (Compound 47; 10 mg/kg) | 26.0 | 3.4 |
| E (Compound 48; 10 mg/kg) | 32.4 | 4.4 |
| F (Compound 50; 10 mg/kg) | 41.2 | 9.7 |

### Example 42

### In Vitro Efficacy: Malaria

TABLE 7

| Compound | IC₅₀ Plasmodium (ng/mL) |
|---|---|
| ^{a}(S,S)-1 | 270 |
| 2 | 8067 |
| 1 | 341 |
| 3 | 8151 |
| 20 | 9175 |
| 44 | 2139 |
| 46 | 4467 |
| 47 | 4851 |
| 48 | 5723 |
| 49 | 2134 |
| 50 | 271 |

^{a}Stereochemistry of Senn et al. product

a. In vitro Parasite Culturing: P. falciparum clone W2/Indochina was grown in continuous culture using RPMI 1640 media containing 10% heat-inactivated type A+ human plasma, sodium bicarbonate (2.4 g/L), HEPES (5.94 g/L) and 4% washed human type A+ erythrocytes. Cultures were gassed with a 90% N₂, 5% O₂ and 5% CO₂ mixture followed by incubation at 37° C.
b. Assay Preparation: Test compounds at 5 mg/mL in DMSO were diluted 1:500 and then serially diluted in duplicate over 11 concentrations. P. falciparum cultures with >70% ring stage parasites were diluted to 0.5-0.7% parasitemia and 1.5% hematocrit in RPMI 1640 media. In 96-well plates a volume of 90 μl/well of parasitized erythrocytes was added on top of 10 μL/well of the test compound. A separate plate containing chloroquine, dihydroartemisinin and atovaquone was added to each set of assay plates as control drugs. A Beckman Coulter Biomek 3000 was used to dispense test compounds, control drugs and parasitized erythrocytes into the microtiter plates. Positive and negative controls were included in each plate. Positive controls consisted of parasitized erythrocytes and negative controls consisted of non-parasitized erythrocytes at 1.5% hematocrit in RPMI 1640 media. Assay plates were placed into a modulator incubator chamber and equilibrated with 90% N₂, 5% O₂ and 5% CO₂ mixture then incubated at 37° C. for 48 hours. After 48 hours, approximately 0.05 μCi of [³H]-hypoxanthine monohydrochloride was added to each well of the assay plates. Plates were put back into the modulator incubation chamber, gassed with the 90% N₂, 5% O₂ and 5% CO₂ mixture then incubated at 37° C. for an additional 24 hours. After 72 hours total incubation time the plates were frozen at −80° C. until later processed for parasite growth determinations.
c. Assay Plate Processing: Assay plates were harvested using a Perkin Elmer FilterMate Harvester. The incorporation of [³H]-hypoxanthine into nucleic acid was measured using a Perkin Elmer Topcount NXT Microplate Scintillation & Luminescence Counter.
d. Data Analysis: Data analysis was performed using a custom database manager (Dataspects, Inc). Nonlinear regression analysis was used to calculate EC₅₀.

### Example 43

### In Vitro Efficacy: Microbial Panel

TABLE 8

| | S. | E. | S. | C. | B. | P. | B. | C. |
|---|---|---|---|---|---|---|---|---|
| Compound | aureus | faecalis | pyogenes | glabrata | subtilis | aeruginosa | anthracis | albicans |
| (S,R)-1 | >1000 | 32 | 8 | >1000 | 8 | >1000 | 4 | >1000 |
| 20 | 1000 | 1000 | 250 | 250 | 250 | >1000 | 1000 | 250 |
| 44 | >1000 | >1000 | >1000 | >1000 | >1000 | >1000 | >1000 | >1000 |
| 45 | 1000 | 1000 | 250 | 500 | 250 | >1000 | 250 | 250 |
| 46 | >1000 | >1000 | 8 | >1000 | 4 | >1000 | >1000 | >1000 |
| 47 | >1000 | >1000 | >1000 | >1000 | >1000 | >1000 | >1000 | >1000 |
| 48 | 500 | 500 | 250 | >1000 | 125 | >1000 | 250 | 1000 |
| 49 | 250 | 500 | 125 | >1000 | 250 | >1000 | 250 | >1000 |
| 50 | 16 | 16 | 8 | 8 | 2 | 4 | 1000 | 16 |

Minimum inhibitory concentrations (MIC) were assessed for S. aureus 13709, S. pyogenes, E. faecalis, C. glabrata, B. subtilis, P. aeruginosa, B. anthracis, and C. albicans using a broth microdilution approach based on CLSI standards and the use of the colorimetric reporter Alamar Blue (Table 8). For susceptibility testing, 10 μL of glycerol stock was suspended in a 10 mL shake flask culture of chemically defined Isosensitest broth (Oxoid) supplemented with 2% w/v glucose. A sample of the shake flask culture was diluted to 1×10⁶ cells/mL in media and added to 96-well test plates (100 μL per well) containing test compounds 93 dispensed in DMSO (2 μL). After an incubation period (30° C.) determined from the strain specific doubling time, a 0.03% w/v aqueous solution of resazurin (10 μL) was added and the plates were allowed to incubate; each well was then scored for dye reduction. The MIC value was taken as the lowest concentration of test compound that inhibits growth such that less than 1% reduction of resazurin (λmax 570 nm) to resorufin (,max 600 nm) was observed.

Adherent cell lines were maintained in Eagle's Minimal Essential Media (Sigma-Aldrich, St. Louis, Mo., USA) with 2 mM glutamine and Earle's Balanced Salt Solution (HyClone, Logan, Utah, USA) adjusted to contain 1.5 g/L sodium bicarbonate, 0.1 mM non-essential amino acids, 1 mM sodium pyruvate and 10% fetal calf serum. Fetal calf serum used in these assays was lot matched throughout. All cultures were maintained under a humidified 5% CO₂ atmosphere at 37° C., had media refreshed twice weekly and were subcultured by trypsinization and resuspension at a ratio of 1:5 each week. Toxicity assays were conducted between passages 10-20. Target compound toxicity was measured by incubating the test compound with the cells for 4 hours, washing the cells and finally treating the cells with Alamar Blue. After 12-24 hours, the fluorescence of the reduced dye was measured. Fluorescence intensity as a function of test compound concentration was fit to the Fermi equation, using non-linear least squares regression analysis, to estimate IC₅₀ values.

### Example 44

### In Vitro Screening

The In Vitro Cell Line Screening Project (IVCLSP), a screening service offered by the Developmental Therapeutics Program of NCI/NIH, utilizes 60 different human tumor cell lines (“NCI-60”), representing leukemia, melanoma and cancers of the lung, colon, brain, ovary, breast, prostate, and kidney. The aim is to prioritize for further evaluation, synthetic compounds or natural product samples showing selective growth inhibition or cell killing of particular tumor cell lines. This screen is unique in that the complexity of a 60 cell line dose response produced by a given compound results in a biological response pattern which can be utilized in pattern recognition algorithms (COMPARE program. See: http://dtp.nci.nih.gov/docs/compare/compare.html). Using these algorithms, it is possible to assign a putative mechanism of action to a test compound, or to determine that the response pattern is unique and not similar to that of any of the standard prototype compounds included in the NCI database (see DTP Overview tab). In addition, following characterization of various cellular molecular targets in the 60 cell lines, it may be possible to select compounds most likely to interact with a specific molecular target.

The screening is a two-stage process, beginning with the evaluation of all compounds against the 60 cell lines at a single dose of 10 uM. The output from the single dose screen is reported as a mean graph and is available for analysis by the COMPARE program. Compounds which exhibit significant growth inhibition are evaluated against the 60 cell panel at five concentration levels.

Information on interpretation of the single-dose data is available at http://dtp.cancer.gov/branches/btb/onedose_interp.html

### Methodology of the In Vitro Cancer Screen.

The human tumor cell lines of the cancer screening panel are grown in RPMI 1640 medium containing 5% fetal bovine serum and 2 mM L-glutamine. For a typical screening experiment, cells are inoculated into 96 well microtiter plates in 100 μL at plating densities ranging from 5,000 to 40,000 cells/well depending on the doubling time of individual cell lines. After cell inoculation, the microtiter plates are incubated at 37° C., 5% CO2, 95% air and 100% relative humidity for 24 hours prior to addition of experimental drugs.

After 24 hours, two plates of each cell line are fixed in situ with TCA, to represent a measurement of the cell population for each cell line at the time of drug addition (Tz). Experimental drugs are solubilized in dimethyl sulfoxide at 400-fold the desired final maximum test concentration and stored frozen prior to use. At the time of drug addition, an aliquot of frozen concentrate is thawed and diluted to twice the desired final maximum test concentration with complete medium containing 50 μg/ml gentamicin. Additional four, 10-fold or ½ log serial dilutions are made to provide a total of five drug concentrations plus control. Aliquots of 100 μl of these different drug dilutions are added to the appropriate microtiter wells already containing 100 μl of medium, resulting in the required final drug concentrations.

Following drug addition, the plates are incubated for an additional 48 hours at 37° C., 5% CO2, 95% air, and 100% relative humidity. For adherent cells, the assay is terminated by the addition of cold TCA. Cells are fixed in situ by the gentle addition of 50 μl of cold 50% (w/v) TCA (final concentration, 10% TCA) and incubated for 60 minutes at 4° C. The supernatant is discarded, and the plates are washed five times with tap water and air dried. Sulforhodamine B (SRB) solution (100 μl) at 0.4% (w/v) in 1% acetic acid is added to each well, and plates are incubated for 10 minutes at room temperature. After staining, unbound dye is removed by washing five times with 1% acetic acid and the plates are air dried. Bound stain is subsequently solubilized with 10 mM trizma base, and the absorbance is read on an automated plate reader at a wavelength of 515 nm. For suspension cells, the methodology is the same except that the assay is terminated by fixing settled cells at the bottom of the wells by gently adding 50 μl of 80% TCA (final concentration, 16% TCA). Using the seven absorbance measurements [time zero, (Tz), control growth, (C), and test growth in the presence of drug at the five concentration levels (Ti)], the percentage growth is calculated at each of the drug concentrations levels. Percentage growth inhibition is calculated as:

[(Ti−Tz)/(C−Tz)]×100 for concentrations for which Ti>/=Tz

[(Ti−Tz)/Tz]×100 for concentrations for which Ti<Tz.

Three dose response parameters are calculated for each experimental agent. Growth inhibition of 50% (GI50) is calculated from [(Ti−Tz)/(C−Tz)]×100=50, which is the drug concentration resulting in a 50% reduction in the net protein increase (as measured by SRB staining) in control cells during the drug incubation. The drug concentration resulting in total growth inhibition (TGI) is calculated from Ti=Tz. The LC50 (concentration of drug resulting in a 50% reduction in the measured protein at the end of the drug treatment as compared to that at the beginning) indicating a net loss of cells following treatment is calculated from [(Ti−Tz)/Tz]×100=−50. Values are calculated for each of these three parameters if the level of activity is reached; however, if the effect is not reached or is exceeded, the value for that parameter is expressed as greater or less than the maximum or minimum concentration tested.

### Example 45

### In Vivo Assay Procedures

a. Acute Toxicity Determination (http://dtp.cancer.gov/branches/btb/acute_tox.html)

Generally, the determination of maximum tolerated dose (MTD) is performed in a way that conserves compound and minimizes the number of animals sacrificed. Thus, a single mouse is given a single injection (IP, IV, SC, IM or PO) of 400 mg/kg (or lower if the compound is anticipated to be extremely potent, e.g. natural products); a second mouse receives a dose of 200 mg/kg and a third mouse receives a single dose of 100 mg/kg. The mice are observed for a period of 2 weeks. They are sacrificed if they lose more than 20% of their body weight or if there are other signs of significant toxicity. If all 3 mice must be sacrificed, the next 3 dose levels (50, 35 and 12.5 mg/kg) are tested in a similar manner. This process is repeated until a tolerated dose is found. This dose is then designated the MTD and is used to calculate the amount of material administered to mice during anti-tumor testing. The mice are allowed ad libitum feed and water. Injections are most commonly administered IP, but SC, PO and IV dosing may be required on occasion. Dose volumes are generally 0.1 mL/10 grams body weight but may be up to 0.2 mL/10 grams of body weight for IP, IV, SC and PO routes.

For the standard hollow fiber assay (HFA), the high and low dose levels are determined using the MTD as determined above using the formula below:

High dose=[MTD×1.5]/4

Low dose=0.67×high dose

The standard vehicle used for both acute toxicity testing and HFA is 10% DMSO in saline/0.05% Tween 80.
b. Hollow Fiber Assay (http://dtp.cancer.gov/branches/btb/hfa.html)

Advancement of potential anticancer agents from identification in the in vitro screen to preclinical development is enhanced with demonstration of in vivo efficacy in one or more animal models of neoplastic disease. Most such models require considerable materials in terms of laboratory animals and test compound as well as substantial amounts of time and cost to determine whether a given experimental agent or series of agents have even minimal anti-tumor activity. The hollow fiber assay described below has demonstrated the ability to provide quantitative indices of drug efficacy with minimum expenditures of time and materials and is currently being utilized as the initial in vivo experience for agents found to have reproducible activity in the in vitro anticancer drug screen.

### Methodology of the Hollow Fiber Assay

A standard panel of 12 tumor cell lines are used for the routine hollow fiber screening of the in vitro actives. These include NCI-H23, NCI-H522, MDA-MB-231, MDA-MB-435, SW-620, COLO 205, LOX, UACC-62, OVCAR-3, OVCAR-5, U251 and SF-295. In addition, alternate lines can be used for specialized testing of compounds on a nonroutine basis. The cell lines are cultivated in RPMI-1640 containing 10% FBS and 2 mM glutamine. On the day preceeding hollow fiber preparation, the cells are given a supplementation of fresh medium to maintain log phase growth. For fiber preparation, the cells are harvested by standard trypsinization technique and resuspended at the desired cell density ((2-10×106 cells/ml). The cell suspension is flushed into 1 mm (internal diameter) polyvinylidene fluoride hollow fibers with a molecular weight exclusion of 500,000 Da. The hollow fibers are heat-sealed at 2 cm intervals and the samples generated from these seals are placed into tissue culture medium and incubated at 370 in 5% CO₂ for 24 to 48 hours prior to implantation. A total of 3 different tumor lines are prepared for each experiment so that each mouse receives 3 intraperitoneal implants (1 of each tumor line) and 3 subcutaneous implants (1 of each tumor line). On the day of implantation, samples of each tumor cell line preparation are quantitated for viable cell mass by a stable endpoint MTT assay so that the time zero cell mass is known. Mice are treated with experimental agents starting on day 3 or 4 following fiber implantation and continuing daily for 4 days. Each agent is administered by intraperitoneal injection at 2 dose levels. The doses are based on the maximum tolerated dose (MTD) determined during prior acute toxicity testing. The fibers are collected from the mice on the day following the fourth compound treatment and subjected to the stable endpoint MTT assay. The optical density of each sample is determined spectrophotometrically at 540 nm and the mean of each treatment group is calculated. The percent net growth for each cell line in each treatment group is calculated and compared to the percent net growth in the vehicle treated controls. A 50% or greater reduction in percent net growth in the treated samples compared to the vehicle control samples is considered a positive result. Each positive result is given a score of 2 and all of the scores are totaled for a given compound. The maximum possible score for an agent is 96 (12 cell lines×2 sites×2 dose levels×2 [score]). A compound is considered for xenograft testing if it has a combined ip+sc score of 20 or greater, a sc score of 8 or greater, or produces cell kill of any cell line at either dose level evaluated. This scoring system has been validated by DCTDC statisticians in CTEP to represent a level of detection expected to score current “standard” agents as active.

c. DTP Human Tumor Xenograft Models (http://dtp.cancer.gov/branches/btb/txm.html)

Advancement of potential cytotoxic anticancer agents from identification through in vitro screens into clinical development generally requires demonstration of in vivo efficacy in one or more animal models of neoplastic disease. After demonstration of activity in the NCI 60-Cell line screen and the hollow fiber assay, compounds are examined for distal site anti-tumor activity in appropriate human tumor xenograft models in nude mice or, where relevant, in rodent tumor models. The specific tumor model employed is based on a number of factors including sensitivity of individual tumor cell lines to the agent. Criteria and selection of compounds for anti-tumor efficacy testing and specific model conditions (tumor types, route of administration, dose, dosing schedule, etc.) are determined on a compound by compound basis by DTP staff after progression through a series of prerequisite assays and an evaluation of available SAR, chemical and biological data. Reviews of human tumor xenograft models and their use in drug development at NCI can be found in the publications listed below.

### All Embodiments

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

Other embodiments are within the claims.

What is claimed is:

1. A compound represented by the following formula 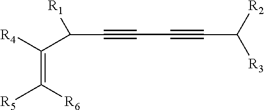
or a salt, ester or prodrug thereof, wherein
R₁ and R₂ vary independently and are selected from the group consisting of a hydrogen, halogen, hydroxyl, acetoxy, thiol, cyanide, azide, chlorosuccinimide, thiocyanate, amine, NHR₇, NR₇R₈, NR₇(C═O)R₈, C(═O)R₇, C(═O)NR₇R₈, NO₂, NH(SO₂)R₇, S(C═O)R₇, SO₂(N)R₇R₈, CO₂R₇, OR₇, OSO₂R₇, NR₇CO₂R₈, OC(═O)R₇, OC(═O)NR₇R₈, piperadino, pyrrolidino, piperazino, azetidino, morpholino, thiomorpholino, (C₁-C₂₀)alkyl, aryl(C₁-C₂₀)alkyl, and aryl, wherein any alkyl or aryl may be optionally substituted with one or more groups selected from R₂, and any alkyl group may optionally include one or more double or triple bonds within its carbon chain; and
R₃, R₄, R₅, R₆, R₇, and R₈ vary independently and are selected from the group consisting of a hydrogen, halogen, (C₁-C₂₀)alkyl, aryl(C₁-C₂₀)alkyl, and aryl, wherein any alkyl or aryl may be optionally substituted with one or more groups selected from R₂, and any alkyl or aryl alkyl group may optionally include one or more double or triple bonds within its carbon chain.

2. The compound of claim 1, wherein said compound is selected from the group consisting of (3S,8R)-8-hydroxyheptadeca-1-en-4,6-diyn-3-yl acetate; (3R,8R)-8-hydroxyheptadeca-1-en-4,6-diyn-3-yl acetate; (3R,8S)-8-hydroxyheptadeca-1-en-4,6-diyn-3-yl acetate; (3S,8R)-8-hydroxyhexadeca-1-en-4,6-diyn-3-yl acetate; (3S,8S)-8-hydroxyhexadeca-1-en-4,6-diyn-3-yl acetate; (3R,8R)-8-hydroxyhexadeca-1-en-4,6-diyn-3-yl acetate; (3R,8S)-8-hydroxyhexadeca-1-en-4,6-diyn-3-yl acetate; (3S,8R)-8-hydroxypentadeca-1-en-4,6-diyn-3-yl acetate; (3S,8S)-8-hydroxypentadeca-1-en-4,6-diyn-3-yl acetate; (3R,8R)-8-hydroxypentadeca-1-en-4,6-diyn-3-yl acetate; (3R,8S)-8-hydroxypentadeca-1-en-4,6-diyn-3-yl acetate; (3S,8R)-8-hydroxytetradeca-1-en-4,6-diyn-3-yl acetate; (3S,8S)-8-hydroxytetradeca-1-en-4,6-diyn-3-yl acetate; (3R,8R)-8-hydroxytetradeca-1-en-4,6-diyn-3-yl acetate; (3R,8S)-8-hydroxytetradeca-1-en-4,6-diyn-3-yl acetate; (3S,8R)-8-hydroxytrideca-1-en-4,6-diyn-3-yl acetate; (3S,8S)-8-hydroxytrideca-1-en-4,6-diyn-3-yl acetate; (3R,8R)-8-hydroxytrideca-1-en-4,6-diyn-3-yl acetate; (3R,8S)-8-hydroxytrideca-1-en-4,6-diyn-3-yl acetate; (3S,8R)-8-hydroxyundeca-1-en-4,6-diyn-3-yl acetate; 8-hydroxy-10-phenyldeca-1-en-4,6-diyn-3-yl acetate; 8-hydroxypentacosa-1-en-4,6-diyn-3-yl acetate; 8-hydroxynonacosa-1-en-4,6-diyn-3-yl acetate; (3S,8R)-heptadeca-1-en-4,6-diyne-3,8-diyl diacetate; (3S,8S)-heptadeca-1-en-4,6-diyne-3,8-diyl diacetate; (3R,8R)-heptadeca-1-en-4,6-diyne-3,8-diyl diacetate; (3R,8S)-heptadeca-1-en-4,6-diyne-3,8-diyl diacetate; (3S,8R)-3-methoxyheptadeca-1-en-4,6-diyn-8-ol; (3S,8S)-3-methoxyheptadeca-1-en-4,6-diyn-8-ol; (3R,8R)-3-methoxyheptadeca-1-en-4,6-diyn-8-ol; (3R,8S)-3-methoxyheptadeca-1-en-4,6-diyn-8-ol; 8-((methylsulfonyl)oxy)heptadeca-1-en-4,6-diyn-3-yl acetate; 8-(tosyloxy)heptadeca-1-en-4,6-diyn-3-yl acetate; 8-acetamidoheptadeca-1-en-4,6-diyn-3-yl acetate; 8-(methylsulfonamido)heptadeca-1-en-4,6-diyn-3-yl acetate; 8-phenoxyheptadeca-1-en-4,6-diyn-3-yl acetate; 8-(acetylthio)heptadeca-1-en-4,6-diyn-3-yl acetate; 8-bromoheptadeca-1-en-4,6-diyn-3-yl acetate; (3S,8R)-8-(4-chlorobenzamido)heptadeca-1-en-4,6-diyn-3-yl acetate; (3S,8R)-8-(3,4-dichlorobenzamido)heptadeca-1-en-4,6-diyn-3-yl acetate; (3S,8R)-8-(4-methylbenzamido)heptadeca-1-en-4,6-diyn-3-yl acetate; (3S,8R)-8-(4-methoxybenzamido)heptadeca-1-en-4,6-diyn-3-yl acetate; (3S,8R)-8-hydroxyundeca-1-en-4,6-diyn-3-yl acetate; (3S,8R)-8-hydroxytricosa-1-en-4,6-diyn-3-yl acetate; (3S,8R)-8-aminoheptadeca-1-en-4,6-diyn-3-yl acetate; (3S,8R)-8-(methylsulfonamido)heptadeca-1-en-4,6-diyn-3-yl acetate; (3S,8R)-8-benzamidoheptadeca-1-en-4,6-diyn-3-yl acetate; (3S,8R)-8-(butylamino)heptadeca-1-en-4,6-diyn-3-yl acetate; (3S,8R)-8-((methylsulfonyl)oxy)heptadeca-1-en-4,6-diyn-3-yl acetate; (3S,8R)-heptadeca-1-en-4,6-diyne-3,8-diol; (3S,8S)-heptadeca-1-en-4,6-diyne-3,8-diol; (3R,8R)-heptadeca-1-en-4,6-diyne-3,8-diol; (3R,8S)-heptadeca-1-en-4,6-diyne-3,8-diol; (3S,8R)-8-aminoheptadeca-1-en-4,6-diyn-3-ol; (3S,8R)-8-(butylamino)heptadeca-1-en-4,6-diyn-3-ol; N-((3S,8R)-3-hydroxyheptadeca-1-en-4,6-diyn-8-yl)benzamide; ethyl((3S,8R)-3-hydroxyheptadeca-1-en-4,6-diyn-8-yl)carbamate; (3S,8R)-8-methoxyheptadeca-1-en-4,6-diyn-3-ol; N-((3S,8R)-3-hydroxyheptadeca-1-en-4,6-diyn-8-yl)acetamide; (3S,8R)-3-hydroxyheptadeca-1-en-4,6-diyn-8-yl acetate; (3S,8R)-8-(5-methylisoxazole-3-carboxamido)heptadeca-1-en-4,6-diyn-3-yl acetate; (3S,8R)-8-(picolinamido)heptadeca-1-en-4,6-diyn-3-yl acetate; (3S,8R)-8-(pyrazine-2-carboxamido)heptadeca-1-en-4,6-diyn-3-yl acetate; (3S,8R)-8-(2-chlorobenzamido)heptadeca-1-en-4,6-diyn-3-yl acetate; (3S,8R)-8-(3-bromobenzamido)heptadeca-1-en-4,6-diyn-3-yl acetate; (3S,8R)-8-(3-methylbenzamido)heptadeca-1-en-4,6-diyn-3-yl acetate; (3S,8R)-8-(4-fluorobenz amido)heptadeca-1-en-4,6-diyn-3-yl acetate; (3S,8R)-8-(3-(trifluoromethyl)benzamido)heptadeca-1-en-4,6-diyn-3-yl acetate; (3S,8R)-8-(furan-2-carboxamido)heptadeca-1-en-4,6-diyn-3-yl acetate; (3S,8R)-8-(3,5-dichlorobenzamido)heptadeca-1-en-4,6-diyn-3-yl acetate; (3S,8R)-8-isothiocyanatoheptadeca-1-en-4,6-diyn-3-yl acetate; (3S,8R)-8-(4-(benzyloxy)butanamido)heptadeca-1-en-4,6-diyn-3-yl acetate; (3S,8R)-8-((cyclopropylmethyl)amino)heptadeca-1-en-4,6-diyn-3-ol; (1S,6R)-6-(butylamino)-1-cyclopropylpentadeca-2,4-diyn-1-ol; (4S,9R,E)-9-(butylamino)octadeca-2-en-5,7-diyn-4-ol; (1S,6R)-6-(butylamino)-1-phenylpentadeca-2,4-diyn-1-ol; (1S,6R)-6-(butylamino)-1-(4-ethoxyphenyl)pentadeca-2,4-diyn-1-ol; (3S,8R)-8-(ethylamino)heptadeca-1-en-4,6-diyn-3-ol; and (3S,8R)-8-benzamido-11-cyclohexylundeca-1-en-4,6-diyn-3-yl acetate.

3. The compound of claim 1 in combination with a pharmaceutically acceptable excipient.

4. A method of treating a patient suffering from, or at risk of acquiring, a protozoal or microbial infection or cancer comprising administering to said patient a therapeutically effective amount of a compound represented by the following formula 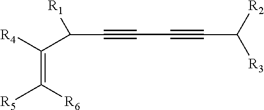
or a salt, ester or prodrug thereof, wherein
R₁ and R₂ vary independently and are selected from the group consisting of a hydrogen, halogen, hydroxyl, acetoxy, thiol, cyanide, azide, chlorosuccinimide, thiocyanate, amine, NHR₇, NR₇R₈, NR₇(C═O)R₈, C(═O)R₇, C(═O)NR₇R₈, NO₂, NH(SO₂)R₇, S(C═O)R₇, SO₂(N)R₇R₈, CO₂R₇, OR₇, OSO₂R₇, NR₇CO₂R₈, OC(═O)R₇, OC(═O)NR₇R₈, piperadino, pyrrolidino, piperazino, azetidino, morpholino, thiomorpholino, (C₁-C₂₀)alkyl, aryl(C₁-C₂₀)alkyl, and aryl, wherein any alkyl or aryl may be optionally substituted with one or more groups selected from R₂, and any alkyl group may optionally include one or more double or triple bonds within its carbon chain; and
R₃, R₄, R₅, R₆, R₇, and R₈ vary independently and are selected from the group consisting of a hydrogen, halogen, (C₁-C₂₀)alkyl, aryl(C₁-C₂₀)alkyl, and aryl, wherein any alkyl or aryl may be optionally substituted with one or more groups selected from R₂, and any alkyl or aryl alkyl group may optionally include one or more double or triple bonds within its carbon chain.

5. The method of claim 4, wherein said compound is selected from the group consisting of (3S,8R)-8-hydroxyheptadeca-1-en-4,6-diyn-3-yl acetate; (3R,8R)-8-hydroxyheptadeca-1-en-4,6-diyn-3-yl acetate; (3R,8S)-8-hydroxyheptadeca-1-en-4,6-diyn-3-yl acetate; (3S,8R)-8-hydroxyhexadeca-1-en-4,6-diyn-3-yl acetate; (3S,8S)-8-hydroxyhexadeca-1-en-4,6-diyn-3-yl acetate; (3R,8R)-8-hydroxyhexadeca-1-en-4,6-diyn-3-yl acetate; (3R,8S)-8-hydroxyhexadeca-1-en-4,6-diyn-3-yl acetate; (3S,8R)-8-hydroxypentadeca-1-en-4,6-diyn-3-yl acetate; (3S,8S)-8-hydroxypentadeca-1-en-4,6-diyn-3-yl acetate; (3R,8R)-8-hydroxypentadeca-1-en-4,6-diyn-3-yl acetate; (3R,8S)-8-hydroxypentadeca-1-en-4,6-diyn-3-yl acetate; (3S,8R)-8-hydroxytetradeca-1-en-4,6-diyn-3-yl acetate; (3S,8S)-8-hydroxytetradeca-1-en-4,6-diyn-3-yl acetate; (3R,8R)-8-hydroxytetradeca-1-en-4,6-diyn-3-yl acetate; (3R,8S)-8-hydroxytetradeca-1-en-4,6-diyn-3-yl acetate; (3S,8R)-8-hydroxytrideca-1-en-4,6-diyn-3-yl acetate; (3S,8S)-8-hydroxytrideca-1-en-4,6-diyn-3-yl acetate; (3R,8R)-8-hydroxytrideca-1-en-4,6-diyn-3-yl acetate; (3R,8S)-8-hydroxytrideca-1-en-4,6-diyn-3-yl acetate; (3S,8R)-8-hydroxyundeca-1-en-4,6-diyn-3-yl acetate; 8-hydroxy-10-phenyldeca-1-en-4,6-diyn-3-yl acetate; 8-hydroxypentacosa-1-en-4,6-diyn-3-yl acetate; 8-hydroxynonacosa-1-en-4,6-diyn-3-yl acetate; (3S,8R)-heptadeca-1-en-4,6-diyne-3,8-diyl diacetate; (3S,8S)-heptadeca-1-en-4,6-diyne-3,8-diyl diacetate; (3R,8R)-heptadeca-1-en-4,6-diyne-3,8-diyl diacetate; (3R,8S)-heptadeca-1-en-4,6-diyne-3,8-diyl diacetate; (3S,8R)-3-methoxyheptadeca-1-en-4,6-diyn-8-ol; (3S,8S)-3-methoxyheptadeca-1-en-4,6-diyn-8-ol; (3R,8R)-3-methoxyheptadeca-1-en-4,6-diyn-8-ol; (3R,8S)-3-methoxyheptadeca-1-en-4,6-diyn-8-ol; 8-((methylsulfonyl)oxy)heptadeca-1-en-4,6-diyn-3-yl acetate; 8-(tosyloxy)heptadeca-1-en-4,6-diyn-3-yl acetate; 8-acetamidoheptadeca-1-en-4,6-diyn-3-yl acetate; 8-(methylsulfonamido)heptadeca-1-en-4,6-diyn-3-yl acetate; 8-phenoxyheptadeca-1-en-4,6-diyn-3-yl acetate; 8-(acetylthio)heptadeca-1-en-4,6-diyn-3-yl acetate; 8-bromoheptadeca-1-en-4,6-diyn-3-yl acetate; (3S,8R)-8-(4-chlorobenzamido)heptadeca-1-en-4,6-diyn-3-yl acetate; (3S,8R)-8-(3,4-dichlorobenzamido)heptadeca-1-en-4,6-diyn-3-yl acetate; (3S,8R)-8-(4-methylbenzamido)heptadeca-1-en-4,6-diyn-3-yl acetate; (3S,8R)-8-(4-methoxybenzamido)heptadeca-1-en-4,6-diyn-3-yl acetate; (3S,8R)-8-hydroxyundeca-1-en-4,6-diyn-3-yl acetate; (3S,8R)-8-hydroxytricosa-1-en-4,6-diyn-3-yl acetate; (3S,8R)-8-aminoheptadeca-1-en-4,6-diyn-3-yl acetate; (3S,8R)-8-(methylsulfonamido)heptadeca-1-en-4,6-diyn-3-yl acetate; (3S,8R)-8-benzamidoheptadeca-1-en-4,6-diyn-3-yl acetate; (3S,8R)-8-(butylamino)heptadeca-1-en-4,6-diyn-3-yl acetate; (3S,8R)-8-((methylsulfonyl)oxy)heptadeca-1-en-4,6-diyn-3-yl acetate; (3S,8R)-heptadeca-1-en-4,6-diyne-3,8-diol; (3S,8S)-heptadeca-1-en-4,6-diyne-3,8-diol; (3R,8R)-heptadeca-1-en-4,6-diyne-3,8-diol; (3R,8S)-heptadeca-1-en-4,6-diyne-3,8-diol; (3S,8R)-8-aminoheptadeca-1-en-4,6-diyn-3-ol; (3S,8R)-8-(butylamino)heptadeca-1-en-4,6-diyn-3-ol; N-((3S,8R)-3-hydroxyheptadeca-1-en-4,6-diyn-8-yl)benzamide; ethyl((3S,8R)-3-hydroxyheptadeca-1-en-4,6-diyn-8-yl)carbamate; (3S,8R)-8-methoxyheptadeca-1-en-4,6-diyn-3-ol; N-((3S,8R)-3-hydroxyheptadeca-1-en-4,6-diyn-8-yl)acetamide; (3S,8R)-3-hydroxyheptadeca-1-en-4,6-diyn-8-yl acetate; (3S,8R)-8-(5-methylisoxazole-3-carboxamido)heptadeca-1-en-4,6-diyn-3-yl acetate; (3S,8R)-8-(picolinamido)heptadeca-1-en-4,6-diyn-3-yl acetate; (3S,8R)-8-(pyrazine-2-carboxamido)heptadeca-1-en-4,6-diyn-3-yl acetate; (3S,8R)-8-(2-chlorobenzamido)heptadeca-1-en-4,6-diyn-3-yl acetate; (3S,8R)-8-(3-bromobenzamido)heptadeca-1-en-4,6-diyn-3-yl acetate; (3S,8R)-8-(3-methylbenzamido)heptadeca-1-en-4,6-diyn-3-yl acetate; (3S,8R)-8-(4-fluorobenz amido)heptadeca-1-en-4,6-diyn-3-yl acetate; (3S,8R)-8-(3-(trifluoromethyl)benzamido)heptadeca-1-en-4,6-diyn-3-yl acetate; (3S,8R)-8-(furan-2-carboxamido)heptadeca-1-en-4,6-diyn-3-yl acetate; (3S,8R)-8-(3,5-dichlorobenzamido)heptadeca-1-en-4,6-diyn-3-yl acetate; (3S,8R)-8-isothiocyanatoheptadeca-1-en-4,6-diyn-3-yl acetate; (3S,8R)-8-(4-(benzyloxy)butanamido)heptadeca-1-en-4,6-diyn-3-yl acetate; (3S,8R)-8-((cyclopropylmethyl)amino)heptadeca-1-en-4,6-diyn-3-ol; (1S,6R)-6-(butylamino)-1-cyclopropylpentadeca-2,4-diyn-1-ol; (4S,9R,E)-9-(butylamino)octadeca-2-en-5,7-diyn-4-ol; (1S,6R)-6-(butylamino)-1-phenylpentadeca-2,4-diyn-1-ol; (1S,6R)-6-(butylamino)-1-(4-ethoxyphenyl)pentadeca-2,4-diyn-1-ol; (3S,8R)-8-(ethylamino)heptadeca-1-en-4,6-diyn-3-ol; and (3S,8R)-8-benzamido-11-cyclohexylundeca-1-en-4,6-diyn-3-yl acetate.

6. The method of claim 4, wherein said compound is in combination with a pharmaceutically acceptable excipient.

7. The method of claim 4, wherein said patient is a human.

8. The method of claim 4, wherein said protozoal infection is caused by Leishmania, Plasmodium, or Trypanosoma.

9. The method of claim 4, wherein said microbial infection is caused by S. aureus, E. faecalis, S. pyogenes, C. glabrata, B. subtilis, P. aeruginosa, B. antracis, or C. albicans.

10. The method of claim 4, wherein cancer is selected from the group consisting of squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, breast cancer, colon cancer, colorectal cancer, endometrial carcinoma, salivary gland carcinoma, kidney cancer, renal cancer, prostate cancer, vulval cancer, thyroid cancer, hepatic carcinoma, and head and neck cancer.

11. A kit comprising
(a) a compound represented by the following formula 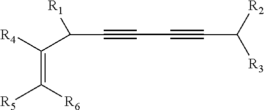
or a salt, ester or prodrug thereof, wherein
R₁ and R₂ vary independently and are selected from the group consisting of a hydrogen, halogen, hydroxyl, acetoxy, thiol, cyanide, azide, chlorosuccinimide, thiocyanate, amine, NHR₇, NR₇R₈, NR₇(C═O)R₈, C(═O)R₇, C(═O)NR₇R₈, NO₂, NH(SO₂)R₇, S(C═O)R₇, SO₂(N)R₇R₈, CO₂R₇, OR₇, OSO₂R₇, NR₇CO₂R₈, OC(═O)R₇, OC(═O)NR₇R₈, piperadino, pyrrolidino, piperazino, azetidino, morpholino, thiomorpholino, (C₁-C₂₀)alkyl, aryl(C₁-C₂₀)alkyl, and aryl, wherein any alkyl or aryl may be optionally substituted with one or more groups selected from R₂, and any alkyl group may optionally include one or more double or triple bonds within its carbon chain; and
R₃, R₄, R₅, R₆, R₇, and R₈ vary independently and are selected from the group consisting of a hydrogen, halogen, (C₁-C₂₀)alkyl, aryl(C₁-C₂₀)alkyl, and aryl, wherein any alkyl or aryl may be optionally substituted with one or more groups selected from R₂, and any alkyl or aryl alkyl group may optionally include one or more double or triple bonds within its carbon chain; and
(b) instructions for the therapeutic administration of said compound to a patient suffering from, or at risk of acquiring, a protozoal or microbial infection or cancer.

12. The kit of claim 11, wherein said patient is a human.

13. The kit of claim 11, wherein said compound is in combination with a pharmaceutically acceptable excipient.

14. The kit of claim 11, wherein said compound is selected from the group consisting of (3S,8R)-8-hydroxyheptadeca-1-en-4,6-diyn-3-yl acetate; (3R,8R)-8-hydroxyheptadeca-1-en-4,6-diyn-3-yl acetate; (3R,8S)-8-hydroxyheptadeca-1-en-4,6-diyn-3-yl acetate; (3S,8R)-8-hydroxyhexadeca-1-en-4,6-diyn-3-yl acetate; (3S,8S)-8-hydroxyhexadeca-1-en-4,6-diyn-3-yl acetate; (3R,8R)-8-hydroxyhexadeca-1-en-4,6-diyn-3-yl acetate; (3R,8S)-8-hydroxyhexadeca-1-en-4,6-diyn-3-yl acetate; (3S,8R)-8-hydroxypentadeca-1-en-4,6-diyn-3-yl acetate; (3S,8S)-8-hydroxypentadeca-1-en-4,6-diyn-3-yl acetate; (3R,8R)-8-hydroxypentadeca-1-en-4,6-diyn-3-yl acetate; (3R,8S)-8-hydroxypentadeca-1-en-4,6-diyn-3-yl acetate; (3S,8R)-8-hydroxytetradeca-1-en-4,6-diyn-3-yl acetate; (3S,8S)-8-hydroxytetradeca-1-en-4,6-diyn-3-yl acetate; (3R,8R)-8-hydroxytetradeca-1-en-4,6-diyn-3-yl acetate; (3R,8S)-8-hydroxytetradeca-1-en-4,6-diyn-3-yl acetate; (3S,8R)-8-hydroxytrideca-1-en-4,6-diyn-3-yl acetate; (3S,8S)-8-hydroxytrideca-1-en-4,6-diyn-3-yl acetate; (3R,8R)-8-hydroxytrideca-1-en-4,6-diyn-3-yl acetate; (3R,8S)-8-hydroxytrideca-1-en-4,6-diyn-3-yl acetate; (3S,8R)-8-hydroxyundeca-1-en-4,6-diyn-3-yl acetate; 8-hydroxy-10-phenyldeca-1-en-4,6-diyn-3-yl acetate; 8-hydroxypentacosa-1-en-4,6-diyn-3-yl acetate; 8-hydroxynonacosa-1-en-4,6-diyn-3-yl acetate; (3S,8R)-heptadeca-1-en-4,6-diyne-3,8-diyl diacetate; (3S,8S)-heptadeca-1-en-4,6-diyne-3,8-diyl diacetate; (3R,8R)-heptadeca-1-en-4,6-diyne-3,8-diyl diacetate; (3R,8S)-heptadeca-1-en-4,6-diyne-3,8-diyl diacetate; (3S,8R)-3-methoxyheptadeca-1-en-4,6-diyn-8-ol; (3S,8S)-3-methoxyheptadeca-1-en-4,6-diyn-8-ol; (3R,8R)-3-methoxyheptadeca-1-en-4,6-diyn-8-ol; (3R,8S)-3-methoxyheptadeca-1-en-4,6-diyn-8-ol; 8-((methylsulfonyl)oxy)heptadeca-1-en-4,6-diyn-3-yl acetate; 8-(tosyloxy)heptadeca-1-en-4,6-diyn-3-yl acetate; 8-acetamidoheptadeca-1-en-4,6-diyn-3-yl acetate; 8-(methylsulfonamido)heptadeca-1-en-4,6-diyn-3-yl acetate; 8-phenoxyheptadeca-1-en-4,6-diyn-3-yl acetate; 8-(acetylthio)heptadeca-1-en-4,6-diyn-3-yl acetate; 8-bromoheptadeca-1-en-4,6-diyn-3-yl acetate; (3S,8R)-8-(4-chlorobenzamido)heptadeca-1-en-4,6-diyn-3-yl acetate; (3S,8R)-8-(3,4-dichlorobenzamido)heptadeca-1-en-4,6-diyn-3-yl acetate; (3S,8R)-8-(4-methylbenzamido)heptadeca-1-en-4,6-diyn-3-yl acetate; (3S,8R)-8-(4-methoxybenzamido)heptadeca-1-en-4,6-diyn-3-yl acetate; (3S,8R)-8-hydroxyundeca-1-en-4,6-diyn-3-yl acetate; (3S,8R)-8-hydroxytricosa-1-en-4,6-diyn-3-yl acetate; (3S,8R)-8-aminoheptadeca-1-en-4,6-diyn-3-yl acetate; (3S,8R)-8-(methylsulfonamido)heptadeca-1-en-4,6-diyn-3-yl acetate; (3S,8R)-8-benzamidoheptadeca-1-en-4,6-diyn-3-yl acetate; (3S,8R)-8-(butylamino)heptadeca-1-en-4,6-diyn-3-yl acetate; (3S,8R)-8-((methylsulfonyl)oxy)heptadeca-1-en-4,6-diyn-3-yl acetate; (3S,8R)-heptadeca-1-en-4,6-diyne-3,8-diol; (3S,8S)-heptadeca-1-en-4,6-diyne-3,8-diol; (3R,8R)-heptadeca-1-en-4,6-diyne-3,8-diol; (3R,8S)-heptadeca-1-en-4,6-diyne-3,8-diol; (3S,8R)-8-aminoheptadeca-1-en-4,6-diyn-3-ol; (3S,8R)-8-(butylamino)heptadeca-1-en-4,6-diyn-3-ol; N-((3S,8R)-3-hydroxyheptadeca-1-en-4,6-diyn-8-yl)benzamide; ethyl((3S,8R)-3-hydroxyheptadeca-1-en-4,6-diyn-8-yl)carbamate; (3S,8R)-8-methoxyheptadeca-1-en-4,6-diyn-3-ol; N-((3S,8R)-3-hydroxyheptadeca-1-en-4,6-diyn-8-yl)acetamide; (3S,8R)-3-hydroxyheptadeca-1-en-4,6-diyn-8-yl acetate; (3S,8R)-8-(5-methylisoxazole-3-carboxamido)heptadeca-1-en-4,6-diyn-3-yl acetate; (3S,8R)-8-(picolinamido)heptadeca-1-en-4,6-diyn-3-yl acetate; (3S,8R)-8-(pyrazine-2-carboxamido)heptadeca-1-en-4,6-diyn-3-yl acetate; (3S,8R)-8-(2-chlorobenzamido)heptadeca-1-en-4,6-diyn-3-yl acetate; (3S,8R)-8-(3-bromobenzamido)heptadeca-1-en-4,6-diyn-3-yl acetate; (3S,8R)-8-(3-methylbenzamido)heptadeca-1-en-4,6-diyn-3-yl acetate; (3S,8R)-8-(4-fluorobenzamido)heptadeca-1-en-4,6-diyn-3-yl acetate; (3S,8R)-8-(3-(trifluoromethyl)benzamido)heptadeca-1-en-4,6-diyn-3-yl acetate; (3S,8R)-8-(furan-2-carboxamido)heptadeca-1-en-4,6-diyn-3-yl acetate; (3S,8R)-8-(3,5-dichlorobenzamido)heptadeca-1-en-4,6-diyn-3-yl acetate; (3S,8R)-8-isothiocyanatoheptadeca-1-en-4,6-diyn-3-yl acetate; (3S,8R)-8-(4-(benzyloxy)butanamido)heptadeca-1-en-4,6-diyn-3-yl acetate; (3S,8R)-8-((cyclopropylmethyl)amino)heptadeca-1-en-4,6-diyn-3-ol; (1S,6R)-6-(butylamino)-1-cyclopropylpentadeca-2,4-diyn-1-ol; (4S,9R,E)-9-(butylamino)octadeca-2-en-5,7-diyn-4-ol; (1S,6R)-6-(butylamino)-1-phenylpentadeca-2,4-diyn-1-ol; (1S,6R)-6-(butylamino)-1-(4-ethoxyphenyl)pentadeca-2,4-diyn-1-ol; (3S,8R)-8-(ethylamino)heptadeca-1-en-4,6-diyn-3-ol; and (3S,8R)-8-benzamido-11-cyclohexylundeca-1-en-4,6-diyn-3-yl acetate.
